SEQUENCE LISTING (28 sequences)

SEQ ID NO: 1 - DNA, 2166 bp, synthetic construct
  source 1..2166: mol_type = other DNA
atggactggacttggcgcattctgtttctggtcgccgctgctactggaactcacgctcaggtgcagctggtcgaatcaggagggggcgtggtccagccaggccgaagcctgaggctggactgcaaggcctccggaatcaccttctcaaacagcggaatgcactgggtgcgccaggctcctgggaaaggactggagtgggtcgcagtgatctggtacgacgggtcaaagcgatactatgctgatagcgtgaaaggcagattcactatttcacgggacaacagcaagaataccctgtttctgcagatgaacagcctgcgggctgaggataccgcagtgtactattgtgcaacaaatgacgattactggggacaggggaccctggtcacagtgagctccgctagtaccaaggggccctcagtgtttcccctggcaccttgctcccgctctactagtgagtcaaccgccgctctgggctgtctggtgaaagattatttccccgaacctgtcacagtgtcatggaatagcggggcactgaccagcggcgtccacacatttcctgccgtgctgcagtctagtgggctgtacagcctgtcaagcgtggtcacagtcccttcctctagtctgggcactaagacctatacatgcaacgtggaccataaaccatccaatactaaggtcgataaaagggtggagtctaagtacggacccccttgcccaccctgtccagcacccgaattcctgggcggaccaagcgtgttcctgtttcctccaaagcccaaagacaccctgatgatctccagaacacctgaggtcacttgcgtggtcgtggacgtgtctcaggaggaccccgaagtccagttcaactggtacgtggatggcgtcgaagtgcacaatgctaagacaaaacccagggaggaacagtttaacagcacatacagggtcgtgtccgtcctgactgtgctgcatcaggactggctgaacggaaaggagtataagtgcaaagtgagcaataaggggctgccatcaagcatcgagaaaaccattagcaaggccaaaggccagccacgggaaccccaggtgtacacactgccccctagccaggaggaaatgactaagaaccaggtcagcctgacctgtctggtgaaaggcttctatccttctgacattgctgtggagtgggaaagtaatggacagccagagaacaattacaagaccacaccacccgtcctggactccgatggctctttctttctgtattccaggctgaccgtggataaatctagatggcaggagggaaacgtctttagctgctccgtgatgcacgaagccctgcacaatcattacacccagaagtctctgagtctgtcactgggaaagcgaggacgaaaaaggagaagcggctccggagccacaaacttctccctgctgaagcaggctggcgacgtggaggaaaatcctggaccaatggtcctgcagactcaggtgtttatctctctgctgctgtggattagtggcgcctacggagagatcgtgctgactcagtcccccgctaccctgtctctgagtcctggcgaacgcgcaaccctgtcttgtcgagcctcacagagcgtgtcctcttacctggcatggtatcagcagaagcctggacaggccccaaggctgctgatctatgatgcctctaaccgggctacagggattcccgcacgcttctccgggtctggcagtggaactgactttactctgaccattagttcactggagccagaagatttcgccgtgtactattgccagcagagctccaattggcccagaacatttgggcagggcactaaggtggagatcaaacggactgtcgcagccccaagcgtgttcatctttcctccatcagacgaacagctgaagtccggaaccgcctctgtggtgtgcctgctgaacaatttctaccccagagaggctaaggtccagtggaaagtggataacgcactgcagagtgggaattcacaggagagcgtgaccgaacaggactccaaggattctacatatagtctgtctagtacactgactctgtccaaagccgactacgagaagcataaagtgtatgcttgcgaagtcactcaccaggggctgcgaagtcccgtcactaagtctttcaatagaggagaatgt

SEQ ID NO: 2 - protein, 722 aa, synthetic construct
MDWTWRILFLVAAATGTHAQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKRGRKRRSGSGATNFSLLKQAGDVEENPGPMVLQTQVFISLLLWISGAYGEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 3 - DNA, 2187 bp, synthetic construct
  source 1..2187: mol_type = other DNA
atggattggacttggagaattctgtttctggtcgccgccgctaccgggacacacgctcaggtgcagctgcagcagtggggggcaggactgctgaagccaagtgagactctgtcactgacctgcgccgtgtacggcggatcattcagcgactactattggaactggatcaggcagccccctggaaaggggctggagtggatcggcgaaattaatcaccggggatcaaccaacagcaatccctccctgaaatctcgcgtgacactgagcctggacacttccaagaaccagttttcactgaaactgcgaagcgtcacagccgctgatactgcagtgtactattgtgccttcggctacagcgactacgagtataattggtttgatccctggggccagggaaccctggtcacagtgagctccgcttccaccaagggaccttctgtgttcccactggcaccctgctccaggtctaccagtgagtcaacagcagccctggggtgtctggtgaaagattatttcccagaacccgtcacagtgagttggaactcaggagcactgaccagcggggtccacacatttcccgccgtgctgcagtctagtggactgtacagcctgtcaagcgtggtcactgtcccatcctctagtctggggactaagacctatacatgcaacgtggaccataaacccagtaataccaaggtcgataaaagagtggagtctaagtacggaccaccatgccctccatgtcctgcaccagaattcctggggggccctagcgtgttcctgtttccccctaagccaaaagacaccctgatgatctcccggactccagaggtcacctgtgtggtcgtggacgtgtctcaggaggaccccgaagtccagttcaactggtacgtggatggcgtcgaagtgcacaatgccaagacaaaacccagggaggaacagtttaatagtacttacagagtcgtgtcagtcctgaccgtgctgcatcaggactggctgaacggaaaggagtataagtgcaaagtgagcaataaggggctgccttcaagcatcgagaaaacaattagcaaggccaaaggccagcctcgggaaccacaggtgtacactctgccacccagccaggaggaaatgactaagaaccaggtcagcctgacatgtctggtgaaagggttctatccctccgacattgctgtggagtgggaatctaatggccagcctgagaacaattacaagaccacacctccagtgctggacagcgatgggtccttctttctgtattctagactgaccgtggataaaagtcggtggcaggagggcaacgtctttagctgctccgtgatgcatgaagccctgcacaatcattacacacagaagtctctgagtctgtcactgggcaagcggggacgcaaaaggagaagcgggtccggcgccactaacttctccctgctgaagcaggctggggacgtggaggaaaatcccggccctatggtcctgcagacacaggtgtttatcagcctgctgctgtggatttccggggcctacggcgagatcgtgctgactcagtccccagctaccctgtctctgagtcccggcgaacgagctaccctgtcttgtagggcatcacagagcatttcctcttacctggcatggtatcagcagaagccaggacaggcacctcgactgctgatctatgatgccagcaaccgcgctactggaattcctgcacgattctccggatctgggagtggcaccgactttactctgaccatcagttcactggagcctgaagatttcgctgtgtactattgccagcagcgatccaactggccactgacatttggacaggggactaatctggagatcaagaggaccgtcgctgcaccttcagtgttcatttttccccctagcgacgaacagctgaaatctggcacagccagtgtcgtgtgtctgctgaacaatttctacccaagggaggctaaggtccagtggaaagtggataacgcactgcagtctggaaatagtcaggagtcagtgacagaacaggacagcaaggattccacttattctctgagctccacactgactctgtccaaagccgactacgagaagcacaaagtctatgcttgcgaagtgacccatcagggcctgtctagtccagtgacaaagtcttttaacagaggagagtgt

SEQ ID NO: 4 - protein, 729 aa, synthetic construct
MDWTWRILFLVAAATGTHAQVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKRGRKRRSGSGATNFSLLKQAGDVEENPGPMVLQTQVFISLLLWISGAYGEIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - DNA, 2187 bp, synthetic construct
  source 1..2187: mol_type = other DNA
atggattggacttggaggattctgtttctggtcgccgccgctacaggaactcacgctcaggtgcagctggtgcagtctggggccgaagtgaagaaacccggcgcttcagtcaaagtgagctgcaaggcatccggatacactttcacctcctattggatgcactgggtgcggcaggcacctggacagggactggagtggatgggggaaattaacccatctaatggcagaacaaactacaacgagaagtttaaaactcgggtgacaatcactgcagacacctccacatctactgcctatatggagctgagctccctgcgctccgaagacactgccgtgtactattgcgccaggggctactatctgtacttcgattattggggccagctgggaactctggtcaccgtgtctagtgcttcaacaaaagggcctagcgtgtttcccctggcaccttcaagcaagagtacatcaggcggaactgccgctctgggctgtctggtgaaggattacttccctgagccagtcaccgtgtcttggaacagtggcgcactgacttccggagtccatacctttcccgccgtgctgcagtcctctggactgtactctctgagttcagtggtcacagtccctagctcctctctggggacccagacatatatttgcaacgtgaatcacaaaccaagtaacactaaggtcgacaagaaagtggaacccaaaagctgtgataagactcatacctgccctccctgtccagcacctgagctgctgggcggcccaagcgtgttcctgtttccacccaagcctaaagacaccctgatgatctcccggaccccagaagtcacatgcgtggtcgtggacgtgtctcacgaggaccccgaagtcaagttcaactggtacgtggatggcgtcgaggtgcataatgctaagacaaaaccacgagaggaacagtacaactccacctatagggtcgtgtctgtcctgacagtgctgcaccaggactggctgaacggaaaggagtataagtgcaaagtgagcaacaaggccctgccagcacccattgagaagacaatcagcaaggcaaaagggcagccaagggaaccccaggtgtacactctgcctccatccagagacgagctgactaaaaaccaggtctctctgacctgtctggtgaaggggttctatccctctgatatcgccgtggagtgggaaagtaatggccagcctgaaaacaattacaagaccacaccccctgtgctggactcagatggcagcttctttctgtatagcaaactgaccgtggacaagtcccgctggcagcagggaaacgtctttagctgctccgtgatgcatgaggccctgcacaatcattacacccagaagtctctgagtctgtcacctgggaaacgaggacgaaagaggagaagcgggtccggagccacaaacttctccctgctgaagcaggctggagatgtggaggaaaatcctgggccaatggtcctgcagactcaggtgtttattagtctgctgctgtggatctcaggagcttacggggacattcagatgacccagagccctagttcactgtctgccagtgtcggagatcgggtgacaatcacttgtcacgctagccagggcatcaggatcaacatcggctggtaccagcagaagcctggcaaagctccaaagctgctgatctaccatggaaccaatctggaagacggggtgccaagcaggttctcaggaagcgggtccggcaccgactttaccctgacaatcagctccctgcagcctgaggatttcgcaacatactattgcgtgcagtacggccagttcccatggacatttggacaggggactaaactgagaattaagaccgtcgcagccccaacagtgagcatctttccaccctctagtgaacagctgacctctggaggggccagtgtggtgtgcttcctgaacaacttctaccccaaggacattaacgtcaagtggaaaatcgatggctcagagcgacagaacggagtgctgaatagctggactgaccaggattccaaagactctacctatagtatgtcaagcactctgaccctgacaaaggatgagtacgaacgccacaatagctatacctgcgaggcaacccacaagacttccacatcccccatcgtgaaatcctttaatagaggcaggtgt

SEQ ID NO: 6 - protein, 729 aa, synthetic construct
MDWTWRILFLVAAATGTHAQVQLVQSGAEVKKPGASVKVSCKASGYTFTSYWMHWVRQAPGQGLEWMGEINPSNGRTNYNEKFKTRVTITADTSTSTAYMELSSLRSEDTAVYYCARGYYLYFDYWGQLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIQMTQSPSSLSASVGDRVTITCHASQGIRINIGWYQQKPGKAPKLLIYHGTNLEDGVPSRFSGSGSGTDFTLTISSLQPEDFATYYCVQYGQFPWTFGQGTKLRIKTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGRC

SEQ ID NO: 7 - DNA, 2292 bp, synthetic construct
  source 1..2292: mol_type = other DNA
atggactggacttggcgcattctgtttctggtggccgccgctactggaactcacgctcaggtgcagctgcaggaatcaggacccggagtggtcaagcccagtggaaccctgtcactgacatgcgccatctccggcggatctattgggagtgggggctcaatccgaagcaccaggtggtggtcatgggtgagacagagcccaggcaagggactggagtggatcggagaaatctaccactcaggaagcactaactataatccttccctgaagtctcgggtgaccattagcctggataaatccagaaaccatttctccctgcggctgaattctgtcactgccgctgacaccgccgtgtactattgtgctcggcaggactacggagattccggggactggtatttcgatctgtgggggaagggcactatggtcaccgtgagctccgctagtaccaaaggcccctcagtgtttcccctggcaccttgctcccgctctacaagtgaatcaactgcagccctgggatgtctggtgaaggactacttccccgaacctgtcacagtgagttggaactcaggagcactgacttctggggtccacacctttcctgccgtgctgcagtctagtgggctgtacagcctgtcaagcgtggtcactgtgccttcctctagtctgggcacaaagacttatacctgcaacgtggatcataaaccaagcaataccaaggtcgacaaaagggtggagtccaagtacggccctccctgcccaccctgtccagcacccgaattcctgggagggcctagcgtgttcctgtttcctccaaagccaaaagataccctgatgatctccagaaccccagaggtcacatgcgtggtcgtggacgtgagccaggaggaccccgaagtccagttcaactggtacgtggacggcgtcgaagtgcacaatgctaagacaaaacctcgggaggaacagtttaacagcacttaccgcgtcgtgtccgtcctgaccgtgctgcatcaggactggctgaacggcaaggagtataagtgcaaagtgagcaataagggactgccatcaagcatcgagaaaacaatttccaaggctaaaggccagccaagggaaccccaggtgtacactctgcccccttctcaggaggaaatgaccaagaaccaggtcagcctgacatgtctggtgaaaggcttttatccctccgatatcgcagtggagtgggaatctaatggacagcctgagaacaattacaagaccacaccacccgtgctggacagcgatggcagcttcttcctgtattcacgcctgaccgtggacaaaagccgatggcaggaggggaacgtcttcagctgctccgtgatgcacgaagccctgcacaatcattacacacagaagtctctgagtctgtcactgggcaagcggggacgcaaaaggagaagcggcagcggggcaactaacttttccctgctgaaacaggccggggatgtggaggaaaatcctggcccaatggtcctgcagacacaggtgttcatctctctgctgctgtggattagtggggcatacggcaactttatgctgacccagccacattctgtcagtgagtcacccgggaagacagtgactatctcctgtacacgatcctctggctctattgccagcaattccgtgcagtggtaccagcagaggcctggcagttcaccaactaccgtgatctatgaggacaaccagaggccctccggagtgcctgatagattctctgggagtattgacagctcctctaattcagccagcctgacagtgagcggactgaagactgaggatgaagccgactactattgccagagttcagatagctccgctgtcgtgttcggctccggaacaaaactgactgtcctggactttggcgtgtactattgtcagcagtacgagttctttgggcagggcaccaaggtccaggtggatatcaaacgcacagtcgctgcaccaagcgtgttcatctttcctccaagcgacgagcagctgaagtctgggaccgctagtgtcgtgtgcctgctgaacaacttctacccccgagaagctaaggtccagtggaaagtggataacgcactgcagtctggcaatagtcaggagtccgtgacagaacaggatagcaaggactccacttattctctgtctagtaccctgacactgagcaaagccgactacgagaagcacaaagtgtatgcttgtgaggtgacccatcagggactgcggagcccagtgacaaaatccttcaataggggagaatgt

SEQ ID NO: 8 - protein, 764 aa, synthetic construct
MDWTWRILFLVAAATGTHAQVQLQESGPGVVKPSGTLSLTCAISGGSIGSGGSIRSTRWWSWVRQSPGKGLEWIGEIYHSGSTNYNPSLKSRVTISLDKSRNHFSLRLNSVTAADTAVYYCARQDYGDSGDWYFDLWGKGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKRGRKRRSGSGATNFSLLKQAGDVEENPGPMVLQTQVFISLLLWISGAYGNFMLTQPHSVSESPGKTVTISCTRSSGSIASNSVQWYQQRPGSSPTTVIYEDNQRPSGVPDRFSGSIDSSSNSASLTVSGLKTEDEADYYCQSSDSSAVVFGSGTKLTVLDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 9 - DNA, 2166 bp, synthetic construct
  source 1..2166: mol_type = other DNA
atgggctggagctgcatcatcctgttcctggtggcaaccgcaacaggagtgcactcccaggtgaagctgctgcagtctggagccgccctggtgaagcctggagcatccgtgaagatgtcttgtaaggccagcggctacacctttacagattattgggtgtcctgggtgaagcagtcccacggcaagtctctggagtggatcggcgagatctacccaaagtctggcaccagcaacttcaatgagaagtttaagggcaaggccaccctgacagtggataagtccacctctacagcctatatggagctgagccggctgacatccgaggactctgccatctactattgcaccggcggcgcctactggggacagggcaccctggtgacagtgagctccgccaagaccacacccccttccgtgtatccactggcaccaggctctgccgcacagaccaatagcatggtgacactgggctgtctggtgaagggctacttccccgagcctgtgaccgtgacatggaacagcggctccctgtctagcggagtgcacacctttccagccgtgctgcagagcgacctgtatacactgtcctctagcgtgaccgtgccttcctctccacggccctccgagaccgtgacatgcaatgtggcccacccagccagctccacaaaggtggacaagaagatcgtgccccgcgattgcggctgtaagccatgcatctgtaccgtgcccgaggtgtctagcgtgttcatctttccacccaagcccaaggatgtgctgaccatcacactgacccctaaggtgacctgcgtggtggtggacatcagcaaggacgatcctgaggtgcagttctcctggtttgtggacgatgtggaggtgcacaccgcccagacacagcctagggaggagcagttcaacagcacctttagatctgtgagcgagctgccaatcatgcaccaggattggctgaatggcaaggagttcaagtgcagggtgaacagcgccgcatttcctgcaccaatcgagaagaccatctccaagacaaagggccgccctaaggccccacaggtgtacacaatccctccacccaaggagcagatggccaaggacaaggtgagcctgacctgtatgatcacagacttctttcctgaggatatcaccgtggagtggcagtggaatggccagcctgccgagaactacaagaatacacagccaatcatgaacaccaatggcagctacttcgtgtattccaagctgaacgtgcagaagtccaattgggaggccggcaacaccttcacctgctctgtgctgcacgagggcctgcacaaccaccacacagagaagtccctgtctcacagcccaggcaagaggggaaggaagaggagatccggctctggcgccaccaatttctctctgctgaagcaggcaggcgatgtggaggagaacccaggacctatgaggtgctccctgcagttcctgggcgtgctgatgttttggatcagcggcgtgtccggcgacgtggtgctgacacagaccccctctatcctgagcaccacaatcggccagagcgtgtccatctcttgtagatcctctcagagcctgctggactccgatggcaacacctacctgtattggttcctgcagaggccaggacagagccctcagcgcctgatctacctggtgtctaatctgaggagcggcgtgcctaacagattcagcggctccggctctggcaccgactttacactgaagatctccggagtggaggcagaggatctgggcgtgtactattgcatgcaggccacccacgacccactgacattcggctctggcaccaagctggagatcaaggcagacgcagcaccaacagtgagcatctttcctccaagctccgagcagctgacctccggcggcgcctctgtggtgtgcttcctgaacaacttctacccaaaggacatcaacgtgaagtggaagatcgatggctctgagcgccagaacggcgtgctgaatagctggacagaccaggattccaaggattctacctatagcatgtctagcacactgaccctgacaaaggacgagtacgagaggcacaattcctatacctgcgaggccacacacaagaccagcacatcccccatcgtgaagtctttcaacagaggcgagtgt

SEQ ID NO: 10 - protein, 722 aa, synthetic construct
MGWSCIILFLVATATGVHSQVKLLQSGAALVKPGASVKMSCKASGYTFTDYWVSWVKQSHGKSLEWIGEIYPKSGTSNFNEKFKGKATLTVDKSTSTAYMELSRLTSEDSAIYYCTGGAYWGQGTLVTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGKRGRKRRSGSGATNFSLLKQAGDVEENPGPMRCSLQFLGVLMFWISGVSGDVVLTQTPSILSTTIGQSVSISCRSSQSLLDSDGNTYLYWFLQRPGQSPQRLIYLVSNLRSGVPNRFSGSGSGTDFTLKISGVEAEDLGVYYCMQATHDPLTFGSGTKLEIKADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 11 - DNA, 2193 bp, synthetic construct
  source 1..2193: mol_type = other DNA
atggattggacttggaggattctgtttctggtcgccgccgcaaccgggactcacgctcaggtgcagctgcagcagtggggcgcaggactgctgaagccttcagagactctgagcctgacctgcgccgtgtacggcggatctttcagtggctactattggagctggatcagacagtcccccgagaagggactggaatggatcggggagattaaccacggcggatacgtgacctataatccttctctggagagtcgggtcacaatttccgtggacacttctaagaaccagttttccctgaaactgagctccgtcacagccgctgacactgcagtgtactattgtgcccgggattacggccctggaaattacgactggtatttcgatctgtggggacgaggcaccctggtcacagtgtctagtgcttctactaaggggccaagcgtgttcccactggcaccctgctcacggagcacctccgaatctacagcagccctgggctgtctggtgaaagattatttcccagagcccgtcacagtgtcatggaacagcggcgcactgacctccggagtccacacatttcccgccgtgctgcagtcaagcgggctgtactctctgtcctctgtggtcaccgtccctagttcaagcctgggcactaagacctatacatgcaacgtggaccataaaccatccaatacaaaggtcgataaacgcgtggaatctaagtacggccctccctgcccaccctgtcctgcaccagagttcctgggagggcctagcgtgttcctgtttcctccaaagccaaaagacaccctgatgatctcccgaactccagaagtcacctgcgtggtcgtggacgtgtctcaggaggaccccgaagtccagttcaactggtacgtggatggagtcgaggtgcacaatgctaagacaaaaccaagggaggaacagtttaactcaacttacagagtcgtgagcgtcctgaccgtgctgcatcaggactggctgaacggaaaggagtataagtgcaaagtgagcaataaggggctgccctcctctatcgaaaaaactattagcaaggctaaaggccagcctcgcgagccacaggtgtacaccctgccccctagccaggaggaaatgaccaagaaccaggtcagcctgacatgtctggtgaaaggcttctatccctctgacatcgcagtggagtgggaaagtaatggacagcctgagaacaattacaagaccacaccacccgtgctggactccgatggctctttctttctgtatagtagactgaccgtggataaatcacggtggcaggaaggaaacgtctttagttgctcagtgatgcacgaggccctgcacaatcattacactcagaagagcctgtccctgtctctgggcaagcggggacgcaaaaggagaagtggatcaggggccaccaacttcagcctgctgaaacaggctggggacgtggaggaaaatcccggccctatggtcctgcagacacaggtgtttatctccctgctgctgtggatttctggggcctacggcgaaatcgtgctgactcagtccccagctaccctgagcctgtccccaggagagcgagctaccctgtcttgtagggcatctcagagtgtgagttcatacctggcatggtatcagcagaagcccggacaggcccctaggctgctgatctatgatgccagcaaccgcgctaccgggattccagcacgattctcaggcagcggcagcggaacagactttactctgaccattagcctggagcccgaagatttcgcagtgtactattgccagcagcgaagcaattggcctccagccctgacatttggcggagggactaaggtggaaatcaaaaggacagtcgctgcacccagcgtgttcatttttcccccttccgacgagcagctgaagagtggaactgcttcagtggtgtgcctgctgaacaatttctaccctagagaagctaaggtccagtggaaagtggataacgcactgcagagtgggaattcacaggaaagcgtgacagagcaggactccaaggattctacttatagtctgagctccacactgactctgagcaaagccgactacgagaagcataaagtgtatgcttgcgaggtcactcaccaggggctgtcaagtccagtcactaaatctttcaatagaggcgaatgt

SEQ ID NO: 12 - protein, 731 aa, synthetic construct
MDWTWRILFLVAAATGTHAQVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKRGRKRRSGSGATNFSLLKQAGDVEENPGPMVLQTQVFISLLLWISGAYGEIVLTQSPATLSLSPGERATLSCRASQSVSSYLAWYQQKPGQAPRLLIYDASNRATGIPARFSGSGSGTDFTLTISLEPEDFAVYYCQQRSNWPPALTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - DNA, 2190 bp, synthetic construct
  source 1..2190: mol_type = other DNA
atggattggacttggaggattctgtttctggtcgccgccgcaactggaacccacgctcaggtgcagctggtgcagtcaggctccgagctgaagaagccaggcgcctccgtgaaggtgtcttgcaaggccagcggctacaccttcacagactatagcatgcactgggtgaggcaggcaccaggacagggcctgaagtggatgggctggatcaacaccgagacaggcgagcccacatacgccgacgacttcaagggcagattcgtgtttagcctggacacatccgtgtctaccgcctatctgcagatcagctccctgaaggccgaggataccgccgtgtactattgtgccaatccatactatgactacgtgtcctactatgccatggattattggggccagggcaccacagtgacagtgtctagcgcctctaccaagggaccaagcgtgttcccactggcaccttgcagcaggtccacatctgagagcaccgccgccctgggatgtctggtgaaggattacttccccgagcctgtgaccgtgagctggaactccggcgccctgacatccggagtgcacacctttcctgccgtgctgcagtcctctggcctgtactctctgagctccgtggtgacagtgccttctagctccctgggcaccaagacatatacctgcaacgtggaccacaagccatctaataccaaggtggataagagggtggagagcaagtacggccctccctgcccaccctgtccagcaccagagtttctgggcggcccatccgtgttcctgtttcctccaaagcctaaggacacactgatgatcagcagaacacctgaggtgacctgcgtggtggtggacgtgtcccaggaggaccccgaggtgcagttcaactggtacgtggatggcgtggaggtgcacaatgccaagaccaagcctcgggaggagcagtttaactccacataccgcgtggtgtctgtgctgaccgtgctgcaccaggactggctgaacggcaaggagtataagtgcaaggtgtctaataagggcctgccatctagcatcgagaagacaatcagcaaggcaaagggacagccacgggagccacaggtgtacaccctgcccccttcccaggaggagatgacaaagaaccaggtgtctctgacctgtctggtgaagggcttctatccaagcgacatcgccgtggagtgggagtccaatggccagcccgagaacaattacaagaccacaccacccgtgctggactctgatggcagcttctttctgtattctaggctgaccgtggataagagcagatggcaggagggcaacgtgttttcctgctctgtgatgcacgaggccctgcacaatcactacacacagaagagcctgtccctgtctctgggcaagaggggaaggaagaggagaagcggctccggagcaaccaacttcagcctgctgaagcaggcaggcgacgtggaggagaatcctggaccaatggtgctgcagacacaggtgtttatcagcctgctgctgtggatctccggcgcctacggcgatatccagatgacccagtccccctcctctctgtctgccagcgtgggcgacagggtgacaatcacctgtaaggcatcccaggacgtgagcaccgcagtggcctggtaccagcagaagcctggcaaggccccaaagctgctgatctattccgcctcttacctgtatacaggagtgcctagccggttcagcggctccggctctggaaccgacttcaccttcaccatcagctccctgcagcccgaggatatcgccacctactattgccagcagcactactccacacctcgcacctttggccagggcacaaagctggagatcaagaccgtggccgccccctccgtgttcatctttcctccatctgacgagcagctgaagagcggaaccgcatccgtggtgtgcctgctgaacaatttctaccctcgcgaggccaaggtgcagtggaaggtggataacgccctgcagtccggcaattctcaggagagcgtgacagagcaggactccaaggattctacctatagcctgtctagcacactgaccctgagcaaggccgactacgagaagcacaaggtgtatgcctgcgaggtcacccaccaggggctgcggtcacccgtcaccaagtccttcaatagaggggaatgc

SEQ ID NO: 14 - protein, 730 aa, synthetic construct
MDWTWRILFLVAAATGTHAQVQLVQSGSELKKPGASVKVSCKASGYTFTDYSMHWVRQAPGQGLKWMGWINTETGEPTYADDFKGRFVFSLDTSVSTAYLQISSLKAEDTAVYYCANPYYDYVSYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKRGRKRRSGSGATNFSLLKQAGDVEENPGPMVLQTQVFISLLLWISGAYGDIQMTQSPSSLSASVGDRVTITCKASQDVSTAVAWYQQKPGKAPKLLIYSASYLYTGVPSRFSGSGSGTDFTFTISSLQPEDIATYYCQQHYSTPRTFGQGTKLEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 15 - DNA, 2196 bp, synthetic construct
  source 1..2196: mol_type = other DNA
atggactggacatggaggattctgtttctggtcgccgctgctactggaacccacgccgaggtcaatctggtcgagtcagggggaggactggtcaagcccggcggatctctgaaagtgagttgcgccgcttcaggcttcacttttagctcctacgccatgtcttgggtcagacagacccctgagaagcggctggaatgggtggctagcatctctagtgggggcaccacatactatccagactcagtgaaaggaaggttcactatcagccgagataacgccaggaatattctgtacctgcagatgtcaagcctgcgaagcgaggacaccgctatgtactattgtgcaagggtgggagggtactatgactctatggattattgggggcagggcattagtgtcactgattcctctgcttcaaccaaggggcccagcgtgtttccactggcaccctgctcaagaagcacttccgagtctaccgcagccctgggctgtctggtgaaagactacttcccagaacccgtcaccgtgagctggaactccggcgcactgacctccggagtccacacatttcctgccgtgctgcagagttcaggactgtactctctgagctccgtggtcacagtgccctctagttcactggggacaaagacttatacctgcaacgtggaccataaacctagcaatactaaggtcgataaacgcgtggagtccaagtacggccctccctgcccaccctgtcctgcaccagaattcctgggcggaccctccgtgttcctgtttcctccaaagcctaaagacaccctgatgatctcccgaacacctgaggtcacttgcgtggtcgtggacgtgtctcaggaggaccccgaagtccagttcaactggtacgtggatggcgtcgaagtgcacaatgctaagacaaaacctcgggaggaacagtttaactccacctaccgcgtcgtgtctgtcctgacagtgctgcatcaggattggctgaacggaaaggagtataagtgcaaagtgagcaataaggggctgccaagctccatcgagaaaacaatttccaaggccaaaggccagcctcgggaaccacaggtgtacactctgcccccttctcaggaggaaatgacaaagaaccaggtcagcctgacttgtctggtgaaagggttctatccatccgacatcgctgtggagtgggaatctaatggccagcccgagaacaattacaagactaccccacccgtgctggactctgatggcagtttctttctgtatagcaggctgaccgtggataaatccagatggcaggagggaaacgtctttagttgctcagtgatgcacgaagccctgcacaatcattacacccagaagagcctgtccctgtctctggggaagcgaggacgcaaaaggagaagtggatcaggggcaacaaacttcagcctgctgaagcaggcaggggacgtggaggaaaatccaggacctatggtcctgcagactcaggtgtttatcagtctgctgctgtggatttcaggagcctatggggatatcgtcctgacccagtcaccagcaagcctggccgtgagtctgggacagcgagcaacaatttcatgtcgagctagcgagtccgtcgacaactacggcgtgagcttcatgaattggtttcagcagaagcccggacagcctccaaaactgctgatctatgctgcaagcaaccagggctccggagtgccagctcgcttctctgggagtggctcaggaaccgatttttccctgaatattcaccccatggaggaagacgatactgcaatgtacttctgccagcagaccaaggaggtgacatggacttttgggggcggaacaaagctggaaatcaaaagagcaactgtcgccgctcccagcgtgttcatctttccccctagtgacgagcagctgaagtctggaacagccagtgtggtgtgcctgctgaacaatttctaccctcgggaagctaaggtccagtggaaagtggataacgcactgcagtccgggaattctcaggagagtgtgaccgaacaggactcaaaggatagcacatattccctgtctagtaccctgacactgtccaaagccgactacgagaagcataaagtgtatgcttgcgaagtcacccaccaggggctgtcaagtccagtcaccaaatcctttaatcggggagaatgt

SEQ ID NO: 16 - protein, 732 aa, synthetic construct
MDWTWRILFLVAAATGTHAEVNLVESGGGLVKPGGSLKVSCAASGFTFSSYAMSWVRQTPEKRLEWVASISSGGTTYYPDSVKGRFTISRDNARNILYLQMSSLRSEDTAMYYCARVGGYYDSMDYWGQGISVTDSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKRGRKRRSGSGATNFSLLKQAGDVEENPGPMVLQTQVFISLLLWISGAYGDIVLTQSPASLAVSLGQRATISCRASESVDNYGVSFMNWFQQKPGQPPKLLIYAASNQGSGVPARFSGSGSGTDFSLNIHPMEEDDTAMYFCQQTKEVTWTFGGGTKLEIKRATVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - DNA, 2205 bp, synthetic construct
  source 1..2205: mol_type = other DNA
atggattggacatggaggattctgtttctggtcgccgccgctactggaactcacgccgaagtgcagctgctggagtcaggaggaggcctggtgcagcccggcggaagcctgaggctgtcctgcgccgcttctggattcacctttagcacatacgggatgcactgggtgagacaggcccctggaaaggggctggagtggctgtcttatatcagtgggggcagctcctacattttctatgcagactccgtgcggggccgctttaccatcagtcgagataactcagaaaatgctctgtacctgcagatgaattctctgcgcgccgaggacacagccgtgtactattgcgccagaattctgcggggagggagcggaatggatctgtggggccagggaactctggtcaccgtgtctagtgcctctaccaagggaccaagcgtgttcccactggctccctcaagcaaatctaccagtggcggaacagcagccctgggctgtctggtgaaggactacttccccgagcctgtcacagtgtcatggaatagcggggctctgaccagcggcgtccatacatttcctgcagtgctgcagtcctctgggctgtactccctgagttcagtggtcactgtcccaagctcctctctgggcactcagacctatatctgcaacgtgaatcacaagcctagcaacaccaaagtcgacaagaaagtggaaccaaagtcctgtgataaaacacatacttgccctccctgtccagcaccagagctgctgggcggcccaagcgtgttcctgtttccacccaagcctaaagacaccctgatgatttctcggactccagaagtcacctgcgtggtcgtggacgtgagccacgaggaccccgaagtcaagttcaacgtcgtgtacgtggatggcgtcgaggtgcataatgccaagacaaaacctagggaggaacagtacaactcaacatatagagtcgtgagcgtcctgactgtgctgcaccaggactggctgaacggaaaggagtataagtgcaaagtgtccaataaggctctgcctgcaccaatcgagaaaacaatttctaaggccaaaggccagcctcgggaaccacaggtgtacactctgcctccatctcgcgacgagctgactaagaatcaggtcagtctgacctgtctggtgaaaggcttttatccctccgatatcgctgtggagtgggaatctaacggacagcctgaaaacaattacaagaccacaccccctgtcctggactccgatggctctttctttctgtattcaaagctgaccgtggataaaagcaggtggcagcagggaaatgtcttctcatgcagcgtgatgcatgaggccctgcacaaccattacacacagaagtccctgtctctgagtcctggcaagcgaggaaggaaaaggagatcagggagcggcgcaactaatttttccctgctgaaacaggcaggcgacgtggaggaaaacccaggacctatggtcctgcagacccaggtgttcatctccctgctgctgtggatttctggggcatacggccagagtgtgctgacccagccaccctccgcctctggaacaccaggacagcgagtgacaatcagctgtactggaagttcaagcaacattggagctgggtacaacgtgtactggtatcagcagctgcccgggacagcacctaagctgctgatctatgggaacattaatcgcccatccggcgtgcccgatcgattcagtggctcaaaaagcggaacttccgcctctctggctatcagcggactgcgctccgaggacgaagcagattactattgcgctgcatgggacaagagtatttcaggactggtcttcggagggggcacaaagctgactgtgctggggcagcctaaagccgctccatccgtgaccctgtttcctccatcctctgaggaactgcaggcaaacaaagccaccctggtgtgcctgatctctgacttctacccaggagccgtcaccgtggcttggaaggcagatagttcaccagtcaaagctggggtggaaactaccacacccagtaagcagtcaaacaacaagtacgcagccagctcctatctgagtctgacccccgagcagtggaagtcacacagaagctattcctgccaggtcacccatgaaggaagcacagtggaaaagacagtcgccccaaccgaatgtagc

SEQ ID NO: 18 - protein, 735 aa, synthetic construct
MDWTWRILFLVAAATGTHAEVQLLESGGGLVQPGGSLRLSCAASGFTFSTYGMHWVRQAPGKGLEWLSYISGGSSYIFYADSVRGRFTISRDNSENALYLQMNSLRAEDTAVYYCARILRGGSGMDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNVVYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGQSVLTQPPSASGTPGQRVTISCTGSSSNIGAGYNVYWYQQLPGTAPKLLIYGNINRPSGVPDRFSGSKSGTSASLAISGLRSEDEADYYCAAWDKSISGLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 19 - DNA, 2010 bp, synthetic construct
  source 1..2010: mol_type = other DNA
atggattggacatggattctgtttctggtcgccgctgctacaagagtgcatagtcaggtgcagctggtggagtcaggaggaggggtcgtgcagcccgggcgatctctgaggctgagttgcgccgcttcaggcttcacctttagctcctacgatatgcactgggtgcggcaggcacctggaaaaggactggaatgggtcgctgtgatctggtatgacggatctaacaaatactatgcagatagtgtgaaggggagattcactattagccgggacaactccaagaataccctgtacctgcagatgaactccctgcgggctgaggataccgcagtgtactattgcgcccgcggctctggaaattgggggttctttgactattgggggcagggcacactggtcaccgtgagcagcgccagtacaaaaggcccctcagtgtttcccctggctccttcaagcaagtcaaccagcggcggaacagcagccctgggatgtctggtgaaggactacttccctgagccagtcaccgtgagttggaactcaggagctctgaccagcggggtccatacatttcctgcagtgctgcagtcctctggactgtactccctgagttcagtggtcaccgtcccaagctcctctctggggactcagacctatatctgcaacgtgaatcacaaaccatccaatacaaaggtcgacaagaaagtggaacccaaatcttgtgataagacacatacttgccctccctgtccagcacctgagctgctgggcggcccaagcgtgttcctgtttccacccaagcctaaagataccctgatgattagccgcactcccgaagtcacctgcgtggtcgtggacgtgtcccacgaggaccccgaagtcaagttcaactggtacgtggacggcgtcgaggtgcataatgctaagacaaaacctagggaggaacagtacaatagcacctatagagtcgtgtccgtcctgacagtgctgcaccaggattggctgaacggaaaggagtataagtgcaaagtgtctaacaaggccctgccagcccccatcgagaagaccattagcaaggctaaagggcagccacgagaaccccaggtgtacacactgcctccatctagggatgagctgactaaaaaccaggtcagtctgacctgtctggtgaaggggttctatcctagcgacatcgcagtggagtgggaatccaatggccagccagaaaacaattacaagaccacaccccctgtgctggacagcgatggctccttctttctgtattcaaaactgactgtggacaagagcaggtggcagcagggaaacgtcttttcctgctctgtgatgcacgaggccctgcacaatcattacacacagaaaagtctgtcactgagcccagggaaacggggccgcaagaggagatccggatctggggcaacaaacttcagcctgctgaagcaggcaggcgacgtggaggaaaatcctggaccaatggattggacttggattctgttcctggtcgctgcagccacaagagtgcattccgacattcagatgactcagtctccaagttcactgagtgcctcagtcggcgatcgcgtgaccatcacatgtcgagcttctcagggaattagtcgctggctggcatggtaccagcagaagcctgaaaaagccccaaagtccctgatctatgctgcaagctccctgcagtcaggagtgcccagccgattcagcggctccggatctgggactgactttactctgaccatttctagtctgcagccagaggatttcgccacctactattgccagcagtacaacacatatcccagaacttttggccagggaacaaaagtggaaatcaagcggactgtcgccgctcctagcgtgttcatctttccaccctcagacgagcagctgaagtccggcaccgcttctgtggtgtgcctgctgaacaatttctaccccagagaggcaaaagtccagtggaaggtggataacgccctgcagtca

SEQ ID NO: 20 - protein, 670 aa, synthetic construct
MDWTWILFLVAAATRVHSQVQLVESGGGVVQPGRSLRLSCAASGFTFSSYDMHWVRQAPGKGLEWVAVIWYDGSNKYYADSVKGRFTISRDNSKNTLYLQMNSLRAEDTAVYYCARGSGNWGFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMDWTWILFLVAAATRVHSDIQMTQSPSSLSASVGDRVTITCRASQGISRWLAWYQQKPEKAPKSLIYAASSLQSGVPSRFSGSGSGTDFTLTISSLQPEDFATYYCQQYNTYPRTFGQGTKVEIKRTVAAPSVFIFPPSDEQLKSGTASVVCLLNNFYPREAKVQWKVDNALQS

SEQ ID NO: 21 - DNA, 2223 bp, synthetic construct
  source 1..2223: mol_type = other DNA
ggatccgccaccatggactggacctggagaatcctgttcctggtggcagcagcaaccggaacacacgcacaggtgcagctggtggagagcggcggcggcgtggtgcagccaggcaggagcctgagactgagctgcgcagcatccggcttcacctttagctcctatggaatgcactgggtgaggcaggcaccaggcaagggcctggagtgggtggccgtgatctggtacgacggctctaacaagtactatgccgatagcgtgaagggcaggttcacaatctctagagacaacagcaagaataccctgtacctgcagatgaattccctgagagccgaggacacagccgtgtactattgtgccagggaccccaggggcgccaccctgtactattactattacggaatggacgtgtggggccagggaaccacagtgacagtgtctagcgcctctaccaagggccctagcgtgtttcccctggccccttgcagcagatccacatctgagagcaccgccgccctgggatgtctggtgaaggactacttccccgagcctgtgacagtgtcttggaacagcggcgccctgacatccggagtgcacacctttcctgccgtgctgcagtcctctggcctgtattctctgagctccgtggtgaccgtgccatctagcaatttcggcacccagacatacacctgcaacgtggaccacaagcccagcaatacaaaggtggataagaccgtggagaggaagtgctgcgtggagtgccctccctgtccagccccacccgtggcaggaccatccgtgttcctgtttcctccaaagcctaaggacacactgatgatcagcagaacaccagaggtgacctgcgtggtggtggacgtgtcccacgaggaccccgaggtgcagtttaactggtacgtggatggcgtggaggtgcacaatgccaagaccaagccaagggaggagcagttcaacagcaccttcagggtggtgtctgtgctgaccgtggtgcaccaggattggctgaacggcaaggagtacaagtgcaaggtgtctaataagggcctgccagcccccatcgagaagacaatcagcaagaccaagggacagccacgggagccacaggtgtataccctgcccccttcccgcgaggagatgacaaagaaccaggtgtctctgacctgtctggtgaagggcttctacccctctgacatcgccgtggagtgggagagcaatggccagcctgagaacaattataagaccacaccacccatgctggactccgatggctctttctttctgtactccaagctgaccgtggataagtctcggtggcagcagggcaacgtgttttcctgctctgtgatgcacgaggccctgcacaatcactacacacagaagagcctgtccctgtctccaggcaagaggggaaggaagaggagaagcggctccggagcaaccaacttcagcctgctgaagcaggcaggcgacgtggaggagaatcctggaccaatggtgctgcagacacaggtgtttatcagcctgctgctgtggatctccggcgcctatggcgacatccagatgacccagagcccctcctctctgtctgccagcgtgggcgatcgggtgacaatcacctgtcgcgcctcccagtctatcaactcctatctggattggtaccagcagaagcctggcaaggccccaaagctgctgatctacgcagccagctccctgcagtccggagtgccctctcgcttcagcggctccggctctggcacagactttacactgaccatctctagcctgcagcctgaggatttcgccacctattactgccagcagtattacagcacacccttcacctttggccctggcacaaaggtggagatcaagaggaccgtggcagcacctagcgtgttcatctttcctccatccgacgagcagctgaagagcggaaccgcatccgtggtgtgcctgctgaacaacttctacccacgcgaggccaaggtgcagtggaaggtggataacgccctgcagagcggcaattcccaggagtctgtgacagagcaggacagcaaggattccacctacagcctgtccaacacactgaccctgagcaaggccgactatgagaagcacaaggtgtacgcctgcgaggtgacacaccagggcctgtcctctcccgtgaccaagtccttcaatcggggcgagtgttgataactcgag

SEQ ID NO: 22 - protein, 733 aa, synthetic construct
MDWTWRILFLVAAATGTHAQVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 23 - DNA, 2199 bp, synthetic construct
  source 1..2199: mol_type = other DNA
atggactggacctggagaatcctgttcctggtggcagcagcaaccggaacacacgcacaggtgcagctggtggagagcggcggcggcgtggtgcagccaggcaggagcctgagactgagctgcgcagcatccggcttcacctttagctcctatggaatgcactgggtgaggcaggcaccaggcaagggcctggagtgggtggccgtgatctggtacgacggctctaacaagtactatgccgatagcgtgaagggcaggttcacaatctctagagacaacagcaagaataccctgtacctgcagatgaattccctgagagccgaggacacagccgtgtactattgtgccagggaccccaggggcgccaccctgtactattactattacggaatggacgtgtggggccagggaaccacagtgacagtgtctagcgcctctaccaagggccctagcgtgtttcccctggccccttgcagcagatccacatctgagagcaccgccgccctgggatgtctggtgaaggactacttccccgagcctgtgacagtgtcttggaacagcggcgccctgacatccggagtgcacacctttcctgccgtgctgcagtcctctggcctgtattctctgagctccgtggtgaccgtgccatctagcaatttcggcacccagacatacacctgcaacgtggaccacaagcccagcaatacaaaggtggataagaccgtggagaggaagtgctgcgtggagtgccctccctgtccagccccacccgtggcaggaccatccgtgttcctgtttcctccaaagcctaaggacacactgatgatcagcagaacaccagaggtgacctgcgtggtggtggacgtgtcccacgaggaccccgaggtgcagtttaactggtacgtggatggcgtggaggtgcacaatgccaagaccaagccaagggaggagcagttcaacagcaccttcagggtggtgtctgtgctgaccgtggtgcaccaggattggctgaacggcaaggagtacaagtgcaaggtgtctaataagggcctgccagcccccatcgagaagacaatcagcaagaccaagggacagccacgggagccacaggtgtataccctgcccccttcccgcgaggagatgacaaagaaccaggtgtctctgacctgtctggtgaagggcttctacccctctgacatcgccgtggagtgggagagcaatggccagcctgagaacaattataagaccacaccacccatgctggactccgatggctctttctttctgtactccaagctgaccgtggataagtctcggtggcagcagggcaacgtgttttcctgctctgtgatgcacgaggccctgcacaatcactacacacagaagagcctgtccctgtctccaggcaagaggggaaggaagaggagaagcggctccggagcaaccaacttcagcctgctgaagcaggcaggcgacgtggaggagaatcctggaccaatggtgctgcagacacaggtgtttatcagcctgctgctgtggatctccggcgcctatggcgacatccagatgacccagagcccctcctctctgtctgccagcgtgggcgatcgggtgacaatcacctgtcgcgcctcccagtctatcaactcctatctggattggtaccagcagaagcctggcaaggccccaaagctgctgatctacgcagccagctccctgcagtccggagtgccctctcgcttcagcggctccggctctggcacagactttacactgaccatctctagcctgcagcctgaggatttcgccacctattactgccagcagtattacagcacacccttcacctttggccctggcacaaaggtggagatcaagaggaccgtggcagcacctagcgtgttcatctttcctccatccgacgagcagctgaagagcggaaccgcatccgtggtgtgcctgctgaacaacttctacccacgcgaggccaaggtgcagtggaaggtggataacgccctgcagagcggcaattcccaggagtctgtgacagagcaggacagcaaggattccacctacagcctgtccaacacactgaccctgagcaaggccgactatgagaagcacaaggtgtacgcctgcgaggtgacacaccagggcctgtcctctcccgtgaccaagtccttcaatcggggcgagtgt

SEQ ID NO: 24 - protein, 733 aa, synthetic construct; identical to SEQ ID NO: 22

SEQ ID NO: 25 - DNA, 2217 bp, synthetic construct
  source 1..2217: mol_type = other DNA
ggatccgccaccatggactggacctggagaatcctgttcctggtggcagcagcaaccggaacacacgcacaggtgcagctggtggagagcggcggcggcgtggtgcagcctggcaggagcctgagactgagctgcgcagcatccggcttcacctttagctcctacacaatgcactgggtgagacaggcaccaggcaagggcctggagtgggtgaccttcatctcttatgacggcaacaataagtactatgccgatagcgtgaagggccggtttaccatctctcgcgacaacagcaagaatacactgtacctgcagatgaactccctgcgggccgaggacaccgccatctactattgcgcaaggacaggatggctgggaccattcgattattggggccagggcaccctggtgacagtgtctagcgccagcacaaagggaccatccgtgtttccactggcaccttcctctaagagcacctccggcggcacagccgccctgggctgtctggtgaaggattacttccctgagccagtgaccgtgtcctggaactctggcgccctgaccagcggagtgcacacatttccagccgtgctgcagagctccggcctgtactccctgtctagcgtggtgaccgtgccttcctctagcctgggcacccagacatatatctgcaacgtgaatcacaagccttccaatacaaaggtggacaagaaggtggagccaaagtcttgtgataagacccacacatgccctccctgtccagcacctgagctgctgggcggcccaagcgtgttcctgtttccacccaagcccaaggacacactgatgatcagccggaccccagaggtgacatgcgtggtggtggacgtgtcccacgaggaccccgaggtgaagttcaactggtacgtggatggcgtggaggtgcacaatgccaagaccaagcctagggaggagcagtacaattctacctatagagtggtgagcgtgctgacagtgctgcaccaggactggctgaacggcaaggagtataagtgcaaggtgtctaataaggccctgccagcccccatcgagaagaccatcagcaaggcaaagggacagccaagggagccacaggtgtacacactgcctccaagcagagacgagctgaccaagaaccaggtgtccctgacatgtctggtgaagggcttctatccctccgatatcgccgtggagtgggagtctaatggccagcctgagaacaattacaagaccacaccccctgtgctggacagcgatggctccttctttctgtatagcaagctgaccgtggacaagtccaggtggcagcagggcaacgtgttttcttgcagcgtgatgcacgaggccctgcacaatcactacacccagaagtccctgtctctgagcccaggcaagaggggaaggaagaggagatccggctctggcgccacaaacttcagcctgctgaagcaggccggcgatgtggaggagaatcctggcccaatggtgctgcagacccaggtgtttatctccctgctgctgtggatctctggcgcctacggagagatcgtgctgacccagtccccaggcacactgagcctgtcccctggagagagggccaccctgtcttgtagagcctctcagagcgtgggctcctcttacctggcctggtatcagcagaagcctggccaggccccaagactgctgatctacggagccttcagccgggccaccggcatccccgaccgcttctccggctctggcagcggcacagacttcaccctgacaatctcccggctggagcctgaggacttcgccgtgtactattgccagcagtatggcagctccccatggacctttggccagggcacaaaggtggagatcaagaggaccgtggcagcaccaagcgtgttcatctttccacccagcgacgagcagctgaagtccggcacagcctctgtggtgtgcctgctgaacaatttctaccctcgggaggccaaggtgcagtggaaggtggataacgccctgcagtctggcaatagccaggagtccgtgaccgagcaggactctaaggatagcacatattccctgtctagcaccctgacactgagcaaggccgattacgagaagcacaaggtgtatgcatgcgaggtgacccaccagggcctgtcctctcccgtgacaaagtcctttaaccgcggcgagtgttgataactcgag

SEQ ID NO: 26 - protein, 731 aa, synthetic construct
MDWTWRILFLVAAATGTHAQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - DNA, 2193 bp, synthetic construct
  source 1..2193: mol_type = other DNA
atggactggacctggagaatcctgttcctggtggcagcagcaaccggaacacacgcacaggtgcagctggtggagagcggcggcggcgtggtgcagcctggcaggagcctgagactgagctgcgcagcatccggcttcacctttagctcctacacaatgcactgggtgagacaggcaccaggcaagggcctggagtgggtgaccttcatctcttatgacggcaacaataagtactatgccgatagcgtgaagggccggtttaccatctctcgcgacaacagcaagaatacactgtacctgcagatgaactccctgcgggccgaggacaccgccatctactattgcgcaaggacaggatggctgggaccattcgattattggggccagggcaccctggtgacagtgtctagcgccagcacaaagggaccatccgtgtttccactggcaccttcctctaagagcacctccggcggcacagccgccctgggctgtctggtgaaggattacttccctgagccagtgaccgtgtcctggaactctggcgccctgaccagcggagtgcacacatttccagccgtgctgcagagctccggcctgtactccctgtctagcgtggtgaccgtgccttcctctagcctgggcacccagacatatatctgcaacgtgaatcacaagccttccaatacaaaggtggacaagaaggtggagccaaagtcttgtgataagacccacacatgccctccctgtccagcacctgagctgctgggcggcccaagcgtgttcctgtttccacccaagcccaaggacacactgatgatcagccggaccccagaggtgacatgcgtggtggtggacgtgtcccacgaggaccccgaggtgaagttcaactggtacgtggatggcgtggaggtgcacaatgccaagaccaagcctagggaggagcagtacaattctacctatagagtggtgagcgtgctgacagtgctgcaccaggactggctgaacggcaaggagtataagtgcaaggtgtctaataaggccctgccagcccccatcgagaagaccatcagcaaggcaaagggacagccaagggagccacaggtgtacacactgcctccaagcagagacgagctgaccaagaaccaggtgtccctgacatgtctggtgaagggcttctatccctccgatatcgccgtggagtgggagtctaatggccagcctgagaacaattacaagaccacaccccctgtgctggacagcgatggctccttctttctgtatagcaagctgaccgtggacaagtccaggtggcagcagggcaacgtgttttcttgcagcgtgatgcacgaggccctgcacaatcactacacccagaagtccctgtctctgagcccaggcaagaggggaaggaagaggagatccggctctggcgccacaaacttcagcctgctgaagcaggccggcgatgtggaggagaatcctggcccaatggtgctgcagacccaggtgtttatctccctgctgctgtggatctctggcgcctacggagagatcgtgctgacccagtccccaggcacactgagcctgtcccctggagagagggccaccctgtcttgtagagcctctcagagcgtgggctcctcttacctggcctggtatcagcagaagcctggccaggccccaagactgctgatctacggagccttcagccgggccaccggcatccccgaccgcttctccggctctggcagcggcacagacttcaccctgacaatctcccggctggagcctgaggacttcgccgtgtactattgccagcagtatggcagctccccatggacctttggccagggcacaaaggtggagatcaagaggaccgtggcagcaccaagcgtgttcatctttccacccagcgacgagcagctgaagtccggcacagcctctgtggtgtgcctgctgaacaatttctaccctcgggaggccaaggtgcagtggaaggtggataacgccctgcagtctggcaatagccaggagtccgtgaccgagcaggactctaaggatagcacatattccctgtctagcaccctgacactgagcaaggccgattacgagaagcacaaggtgtatgcatgcgaggtgacccaccagggcctgtcctctcccgtgacaaagtcctttaaccgcggcgagtgt

SEQ ID NO: 28 - protein, 731 aa, synthetic construct; identical to SEQ ID NO: 26

(19) United States
(12) Patent Application Publication
(10) Pub. No.: US 2024/0189405 A1
(43) Pub. Date: Jun. 13, 2024

(54) DNA Monoclonal Antibodies Targeting Checkpoint Molecules

(71) Applicants: The Trustees of the University of Pennsylvania; The Wistar Institute of Anatomy and Biology, Philadelphia, PA (US); Inovio Pharmaceuticals, Inc., Plymouth Meeting, PA (US)

(72) Inventors: David B. Weiner, Merion, PA (US); Kar Muthumani, Collegeville, PA (US); Niranjan Sardesai, Blue Bell, PA (US)

(21) Appl. No.: 18/413,721

(22) Filed: Jan. 16, 2024

Related U.S. Application Data
(63) Continuation of application No. 16/098,925, filed on Nov. 5, 2018, now Pat. No. 11,896,654.
(60) Provisional application No. 62/332,386, filed on May 5, 2016.

(51) Int. Cl. A61K 39/00; A61K 31/7088; A61K 39/395; A61P 35/00; C07K 16/28; C12N 15/85

(52) U.S. Cl. CPC A61K 39/001129; A61K 31/7088; A61K 39/395; A61P 35/00; C07K 16/2803; C07K 16/2818; C07K 16/2878; C12N 15/85; A61K 2039/505; A61K 2039/53

(57) ABSTRACT

Disclosed herein is a composition including a recombinant nucleic acid sequence that encodes an antibody or fragment thereof that targets an immune checkpoint molecule. The disclosure also provides a method of preventing and/or treating disease in a subject using said composition and method of generation.

### CROSS REFERENCE TO RELATED APPLICATION

This application claims priority to and the benefit of U.S. provisional application No. 62/332,386, filed May 5, 2016, the content of which is incorporated herein in its entirety.

### TECHNICAL FIELD

The present invention relates to a composition comprising a recombinant nucleic acid sequence for generating one or more synthetic antibodies, including antibodies targeting the immune checkpoint molecules (e.g., PD-1, PD-L1, LAG-3, GITR, CD40, OX40, CTLA-4, TIM-3, 4-1BB, and combinations and functional fragments thereof), in vivo, and a method of preventing and/or treating cancer, infectious diseases and other conditions in a subject by administering said composition.

### BACKGROUND

Vaccines are used to stimulate an immune response in an individual to provide protection against and/or treatment for a particular disease. Some vaccines include an antigen to induce the immune response. Some antigens elicit a strong immune response while other antigens elicit a weak immune response. A weak immune response to an antigen can be strengthened by including an adjuvant in the vaccine. Adjuvants come in many different forms, for example, aluminum salts, oil emulsions, sterile constituents of bacteria or other pathogens, cytokines, and so forth.

Programmed cell death protein 1 also known as PD-1 is a 288 amino acid cell surface protein molecule that in humans is encoded by the PDCD1 gene. This protein is expressed in pro-B cells and is thought to play a role in their differentiation. PD1 is a type I membrane protein of 268 amino acids and a member of the extended CD28/CTLA-4 family of T cell regulators. The protein's structure includes an extracellular IgV domain followed by a transmembrane region and an intracellular tail. The intracellular tail contains two phosphorylation sites located in an immunoreceptor tyrosine-based inhibitory motif and an immunoreceptor tyrosine-based switch motif, which suggests that PD-1 negatively regulates TCR signals.

PD-1 has two ligands, PD-L1 and PD-L2, which are members of the B7 family. PD-L1 protein is upregulated on macrophages and dendritic cells (DC) in response to LPS and GM-CSF treatment, and on T cells and B cells upon TCR and B cell receptor signaling, whereas in resting mice, PD-L1 mRNA can be detected in the heart, lung, thymus, spleen, and kidney. PD-L1 is expressed on almost all murine tumor cell lines, including PA1 myeloma, P815 mastocytoma, and B16 melanoma upon treatment with IFN-γ. PD-L2 expression is more restricted and is expressed mainly by DCs and a few tumor lines.

There are studies suggesting that PD-1 and its ligands negatively regulate immune responses. PD-1 knockout mice have been shown to develop lupus-like glomerulonephritis and dilated cardiomyopathy on the C57BL/6 and BALB/c backgrounds, respectively. In vitro, treatment of anti-CD3 stimulated T cells with PD-L1-Ig results in reduced T cell proliferation and IFN-γ secretion. It appears that upregulation of PD-L1 may allow cancers to evade the host immune system. PD-L1 expression has been shown to correlate inversely with intraepithelial CD8+ T-lymphocyte count, suggesting that PD-L1 on tumor cells may suppress antitumor CD8+ T cells.

LAG3 and TIM3 are some of the many receptor molecules on the surface of T lymphocytes that exert inhibitory functions.

T cell immunoglobulin domain and mucin domain 3 (TIM-3; also known as HAVCR2), is a human protein that is encoded by the HAVCR2 gene. TIM-3 is a protein surface receptor expressed by activated T cells of the IFNgamma-producing CD4 ThI and CD8 cytotoxic T cells. Its ligand is galectin-9 which is abundantly expressed in the tumor microenvironment and induces cell death and T cell exhaustion of CD4 and CD8 T cells. Evidence of Tim-3 as a key immune checkpoint in either tumor or viral-induced immune suppression comes from demonstration that Tim-3 expressing CD8 T cells are the most suppressed or dysfunctional population of CD8 T cell in preclinical models.

Lymphocyte activation gene 3 (Lag-3 also known as CD223) is a member of the Ig superfamily that is expressed only on activated and tolerized T cells that binds MHC-II molecules and which is known to transduce inhibitory signals. LAG-3 is markedly upregulated on exhausted T cells compared to effector or memory T cells. LAG-3 negatively regulates T cell expansion by inhibiting T cell receptor induced calcium fluxes, thus controlling the size of the T cell memory pool. Studies have shown that in the context of cancer, LAG3 is upregulated on TILs and blockade of LAG-3 can enhance antitumor T cell immune responses. Blockage of LAG-3 in a viral chronic model that evokes CD8 T cells exhaustion, can invigorate the CD8 T cell responses.

Collectively, these aforementioned proteins, along with other inhibitory receptors, such as CTLA-4, are important players in the CD8 T cell exhaustion that takes place in chronic immune conditions such as chronic viral infection and cancer in both experimental models and humans. These known features and function of PD1-1, CTLA-4, TIM-3 and LAG-3 make them an appealing target for immune modulation in vaccine settings.

Thus, there is a need in the art for improved compositions and methods that target immune checkpoint molecules for the treatment of cancer, infectious diseases, and other conditions.

### SUMMARY OF THE PREFERRED EMBODIMENTS

In one aspect, the invention provides a composition for generating a synthetic antibody in a subject comprising one or more nucleic acid molecules encoding one or more synthetic antibodies or fragments thereof, wherein the one or more antibodies or fragments target at least one immune checkpoint molecule.

In one embodiment, the at least one immune checkpoint molecule is selected from the group consisting of PD-1, LAG-3, PD-L1, GITR, CD40, OX40, CTLA-4, TIM-3, 4-1BB, and a combination thereof.

In one embodiment, the composition comprises a nucleotide sequence encoding a cleavage domain.

In one embodiment, the composition comprises a nucleotide sequence encoding a variable heavy chain region and a variable light chain region of the antibody.

In one embodiment, the composition comprises a nucleotide sequence encoding a constant heavy chain region and a constant light chain region of human IgG1κ.

In one embodiment, the composition comprises a nucleotide sequence encoding a polypeptide comprising a variable heavy chain region of the antibody; a constant heavy chain region of human IgG1κ; a cleavage domain; a variable light chain region of the antibody; and a constant light chain region of IgG1κ.

In one embodiment, the composition comprises a nucleotide sequence that encodes a leader sequence.

In one embodiment, the composition comprises a nucleotide sequence encoding at least one amino acid sequence of SEQ ID NOs: 2, 4, 6, 8, 10, 12, 14, 16, 18, 20, 22, 24, 26, and 28.

In one embodiment, the composition comprises at least one nucleic acid sequence of SEQ ID NOs: 1, 3, 5, 7, 9, 11, 13, 15, 17, 19, 21, 23, 25, and 27.

In one embodiment, the one or more nucleic acid molecules are engineered to be in an expression vector.

In one embodiment, the composition further comprises a nucleotide sequence encoding an antigen.

In one embodiment, the composition further comprises a pharmaceutically acceptable excipient.

In another aspect, the invention provides a method of treating a disease in a subject, the method comprising administering to the subject at least one composition of the invention.

In one embodiment, the disease is cancer. In another embodiment, the disease is an infectious disease.

In another aspect, the invention provides a method for increasing an immune response in a subject in need thereof, the method comprising administering a composition of the invention to the subject.

In one embodiment, administering the composition comprises an electroporating step.

In another aspect, the invention provides a method of increasing an immune response in a subject in need thereof by administering a combination of a synthetic antigen and an immune checkpoint inhibitor, wherein the immune checkpoint inhibitor is a synthetic antibody, wherein the administering step comprises: administering to the subject a prime vaccination and a boost vaccination of synthetic antigen, and subsequent to the boost vaccination, administering to the subject an immune checkpoint inhibitor.

In one embodiment, the method further comprises a step of administering to the subject a subsequent boost vaccination of the synthetic antigen. In one embodiment, any of the administering steps include delivering electroporation to the site of administration.

### BRIEF DESCRIPTION OF THE DRAWINGS

FIG. 1 provides an image depicting the design of DNA based monoclonal antibodies (dMAb).

FIG. 2 provides a series of images showing (FIG. 2A) a non-limiting list of targets using dMAb technology, and (FIG. 2B) transfection supernatant IgG concentration (μg/mL).

FIG. 3 provides a series of images demonstrating the construction of PD-1 and LAG-3 dMAb plasmids and confirmation of in vitro and in vivo IgG production. (FIG. 3A) construction of dMAb plasmids. (FIG. 3B) confirmation of in vitro IgG production. (FIG. 3C) confirmation of in vivo IgG production.

FIG. 4 provides a series of images showing in vivo produced IgG following PD-1 or LAG-3 dMAb plasmid administration bind specifically to their targets. (FIG. 4A) binding to hrPD-1 or hrLAG-3. (FIG. 4B) western blots against PD-1 or LAG-3 using corresponding dMAb produced in vivo. (FIG. 4C) FACS showing binding to PD-1 or LAG-3 using pVAX1 sera, dMAb sera, or positive control.

FIG. 5 provides a series of images showing that LAG-3 dMAb impedes tumor growth, improves survival, and promotes a less inhibitory tumor microenvironment. (FIG. 5A) tumor challenge experiment showing improved survival and decreased tumor size following administration of LAG-3 dMAb. (FIG. 5B) graph showing the percentage of CD25+ LAG3+ cells after treatment with pVax-1 (control) or LAG3 dMAb.

FIG. 6 provides a series of images showing dMAb antibodies bind to activated T cells. FACS analysis of PD-1+ T-cells (FIG. 6A) unstimulated and (FIG. 6B) PHA stimulated, for the various conditions depicted.

FIG. 7 provides an image showing LAG-3 dMAb IgG concentration in nude mice.

FIG. 8 provides an image showing LAG-3 dMAb binds to LAG-3 in an ELISA assay.

FIG. 9 provides an image showing a western blot for LAG-3, demonstrating the specificity of LAG-3 dMAb for human LAG-3.

FIG. 10 provides a series of images showing dMAb antibodies bind to activated T cells. FACS analysis of LAG-3+ T-cells (FIG. 10A) unstimulated and (FIG. 10B) PHA stimulated, for the various conditions depicted.

FIG. 11 provides a series of images showing dMAb antibodies block activated Treg cells.

FIG. 12 provides a series of images showing GITR dMAb expression in nude mice. (FIG. 12A) pVax control treatment. (FIG. 12B) GITR dMAb treatment. (FIG. 12C) ELISA showing binding of GITR dMAb to GITR.

FIG. 13 provides a series of images showing FACS analysis of GITR+ T-cells. (FIG. 13A) unstimulated versus (FIG. 13B) PHA stimulated cells, for the various conditions depicted.

FIG. 14 provides a series of images showing OX40 dMAb production in nude mice.

FIG. 15 provides a series of images showing 4-1BB dMAb production in nude mice, and ELISA assay showing specific binding. (FIG. 15A) pVax control treatment. (FIG. 15B) 4-1BB dMAb treatment. (FIG. 15C) ELISA showing binding of 4-1BB dMAb to 4-1BB.

FIG. 16 provides a graph showing anti-CTLA-4 antibodies ipilimumab and tremelimumab expression in 293T cells in vitro.

FIG. 17 provides a series of images showing in vivo expression and binding of anti-CTLA-4 antibodies ipilimumab and tremelimumab in Balb/c mice.

FIG. 18 provides a graph showing in vivo expression of ipilimumab and tremelimumab in Balb/c mice. Delivery was one shot of dMAb (100 μg of DNA at one site). The graph shows mouse anti-human antibody immune response and clearance.

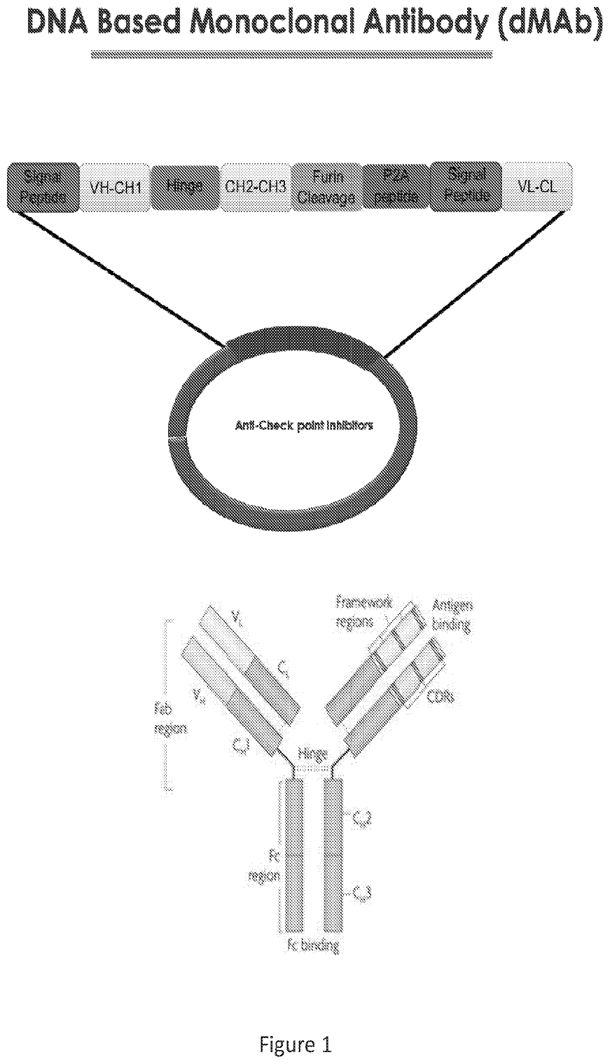

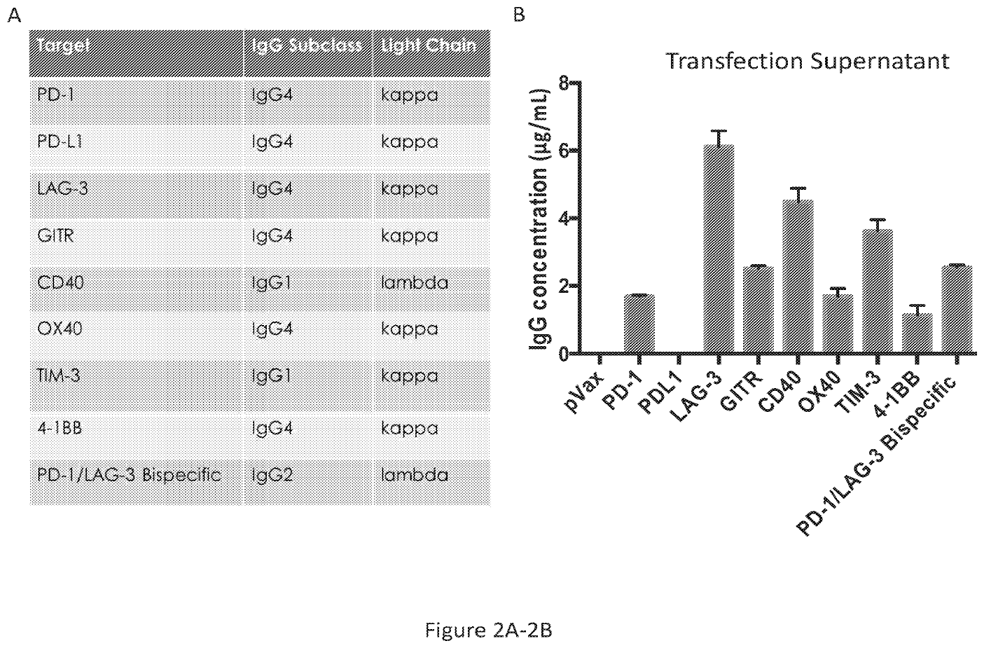

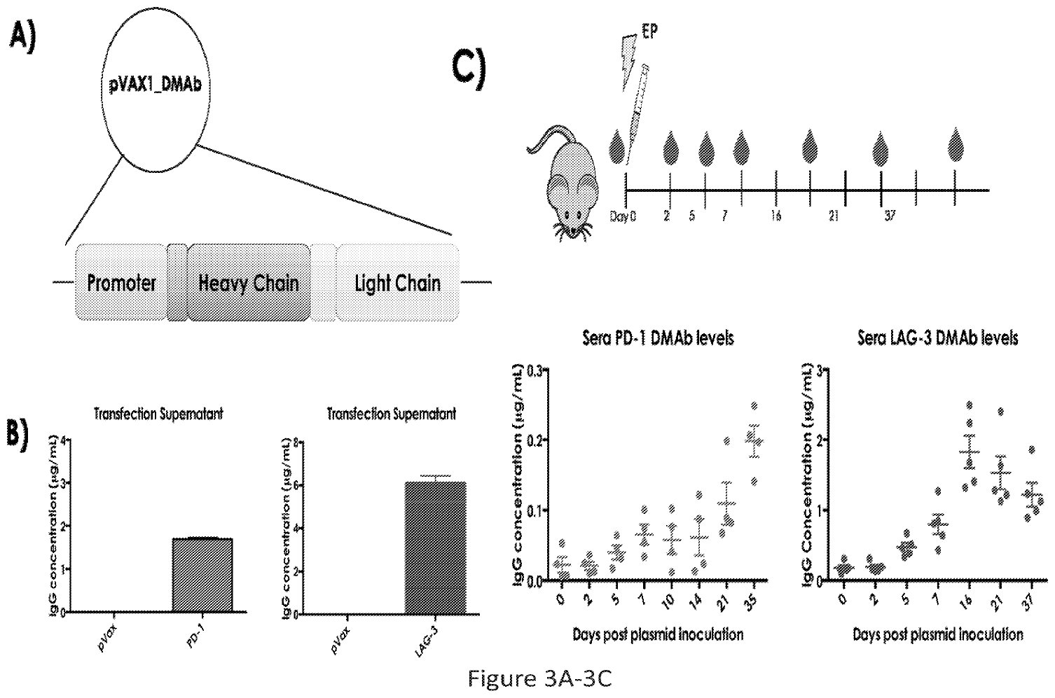

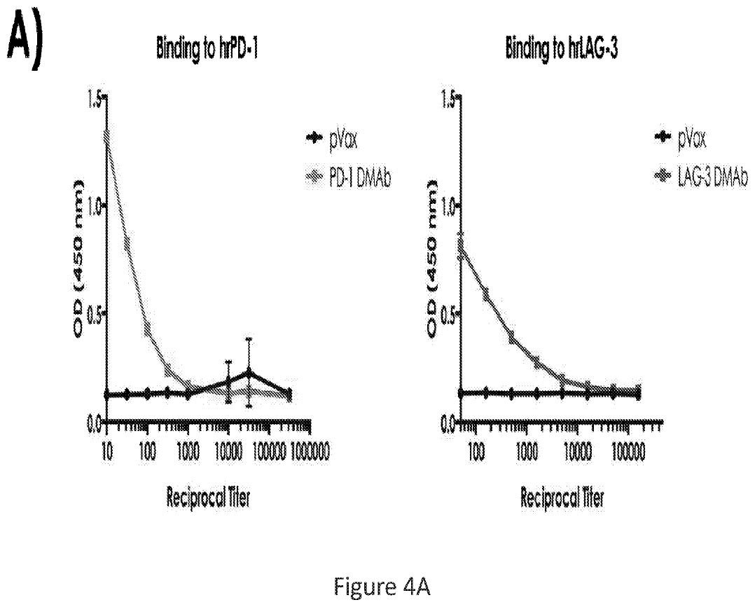

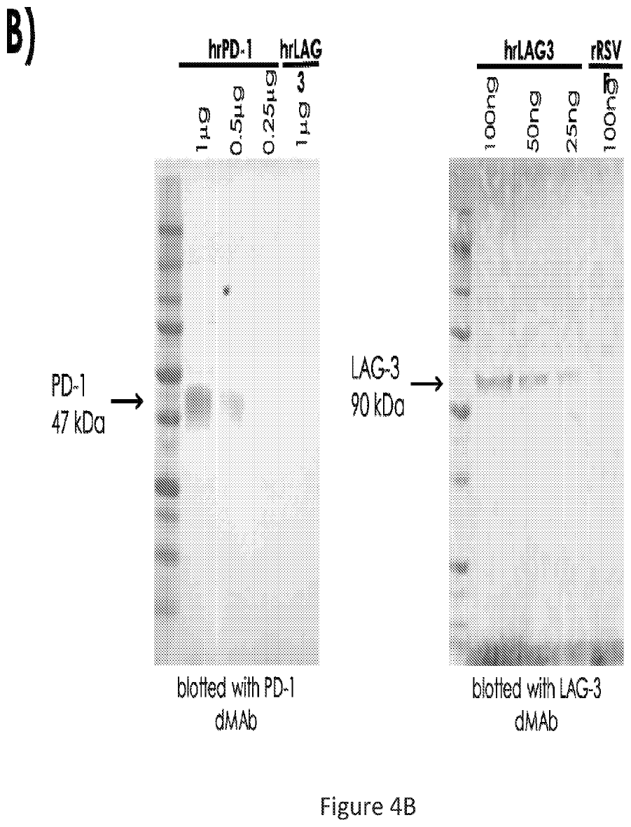

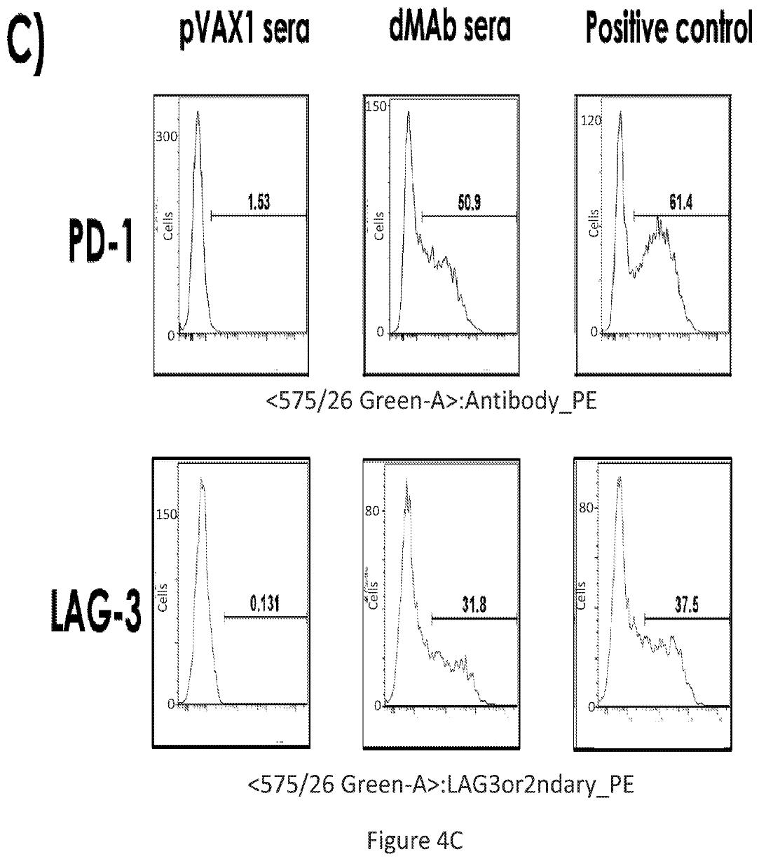

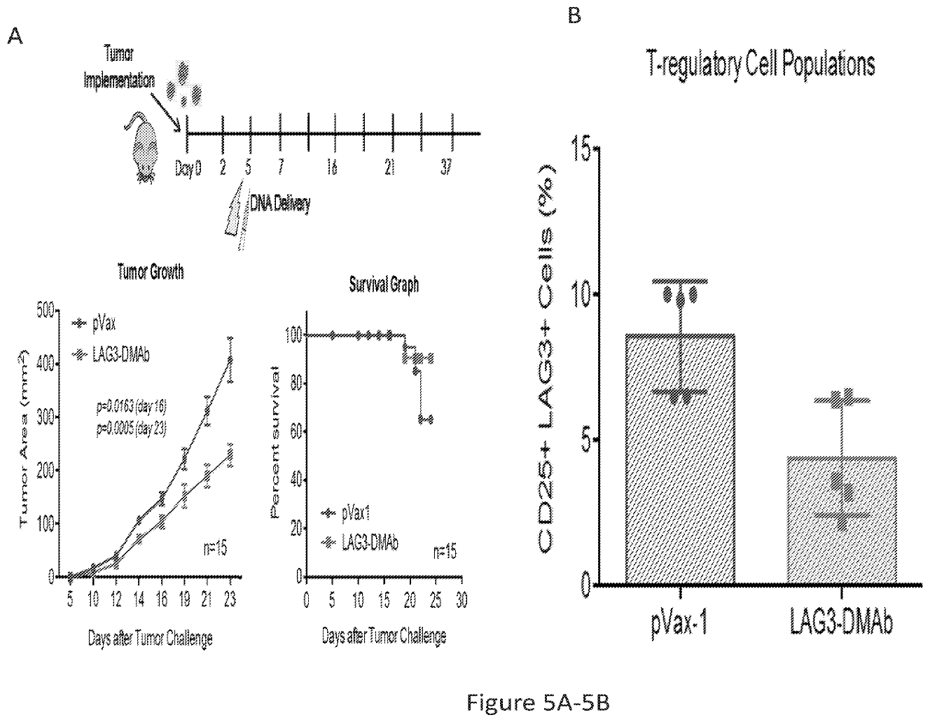

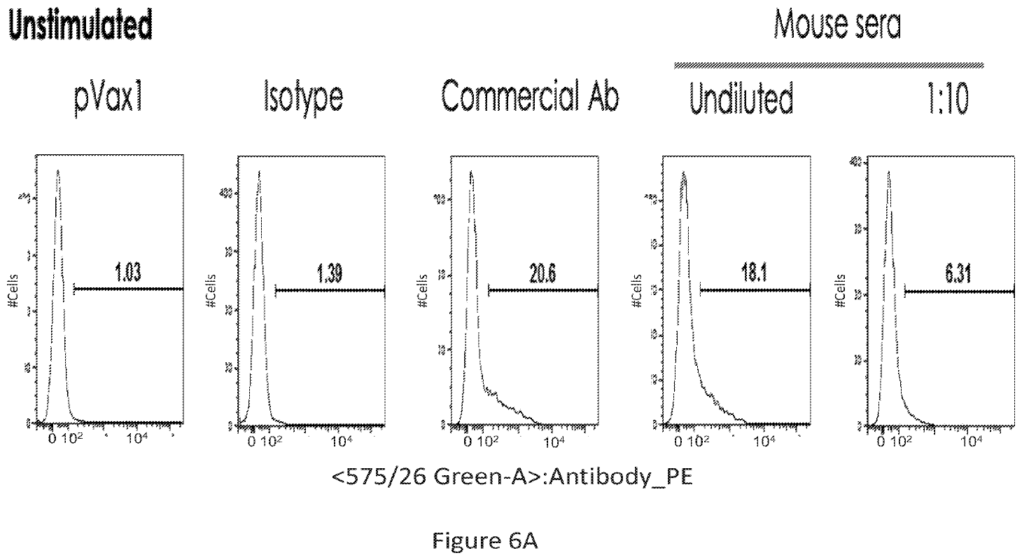

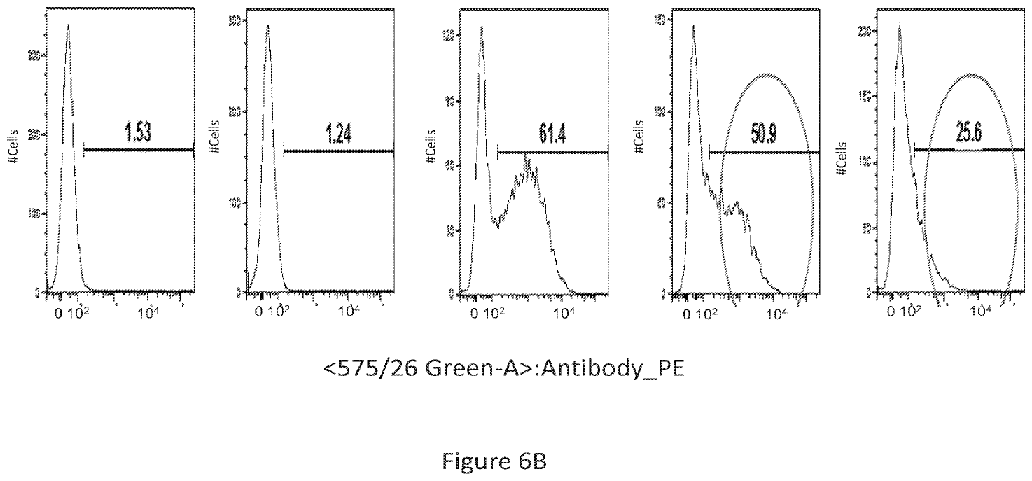

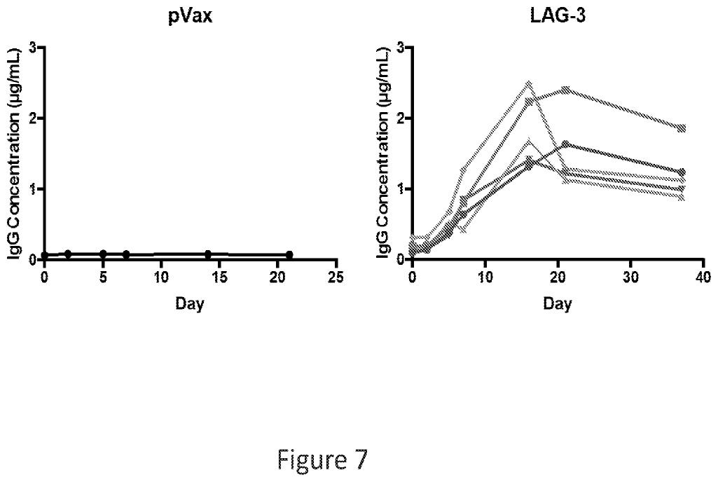

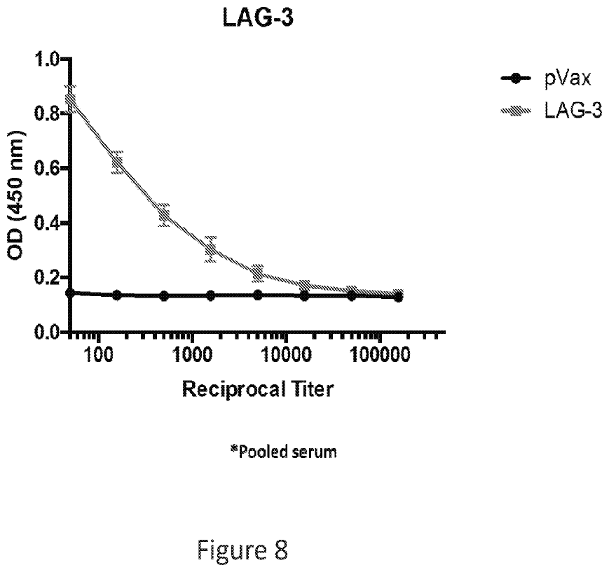

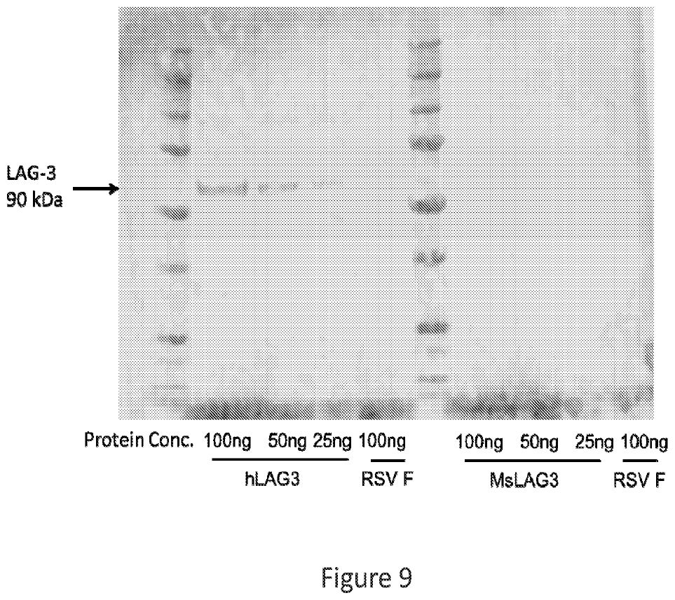

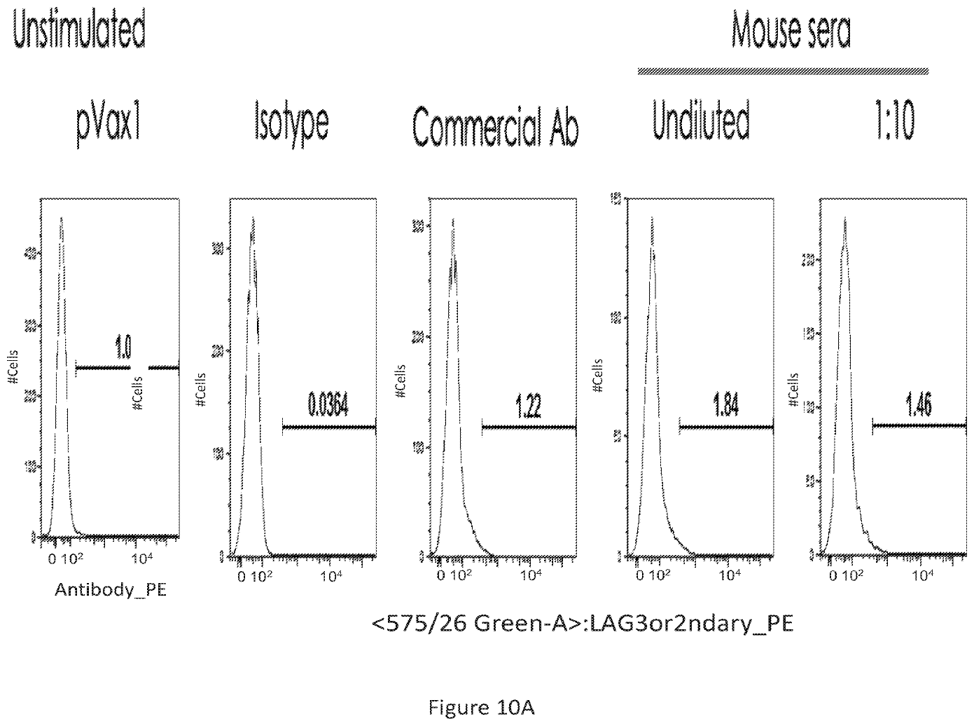

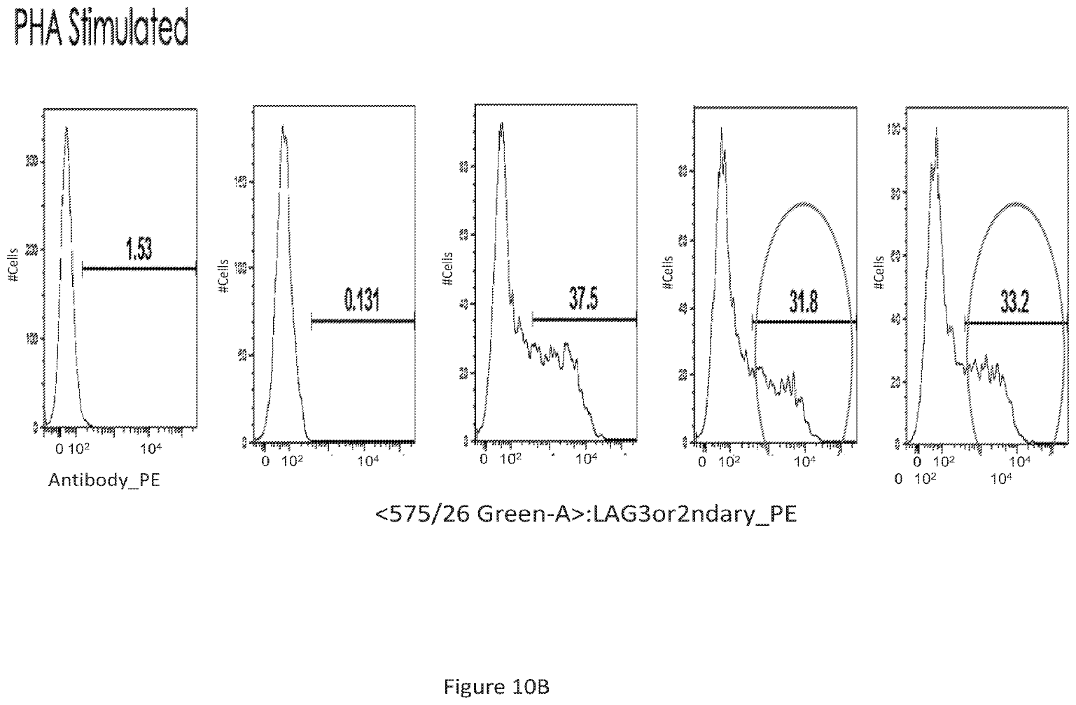

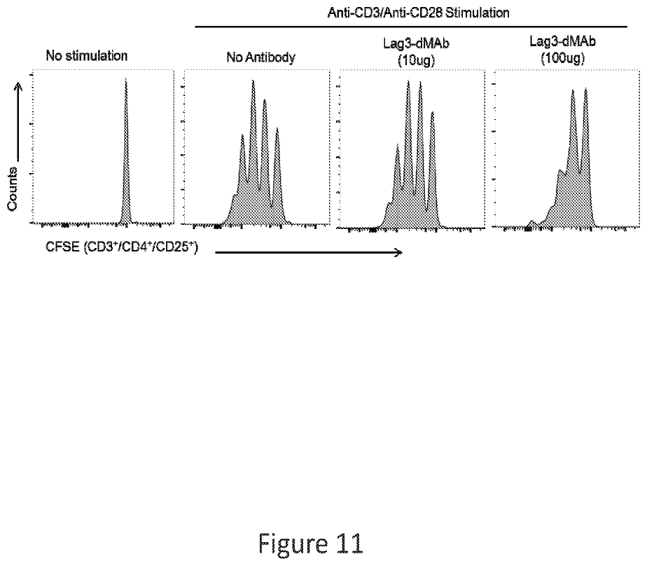

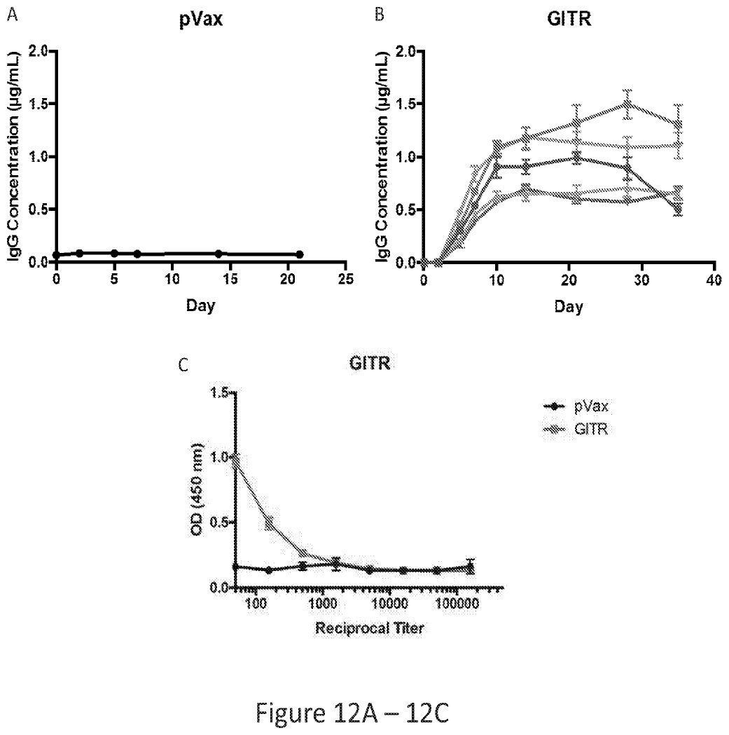

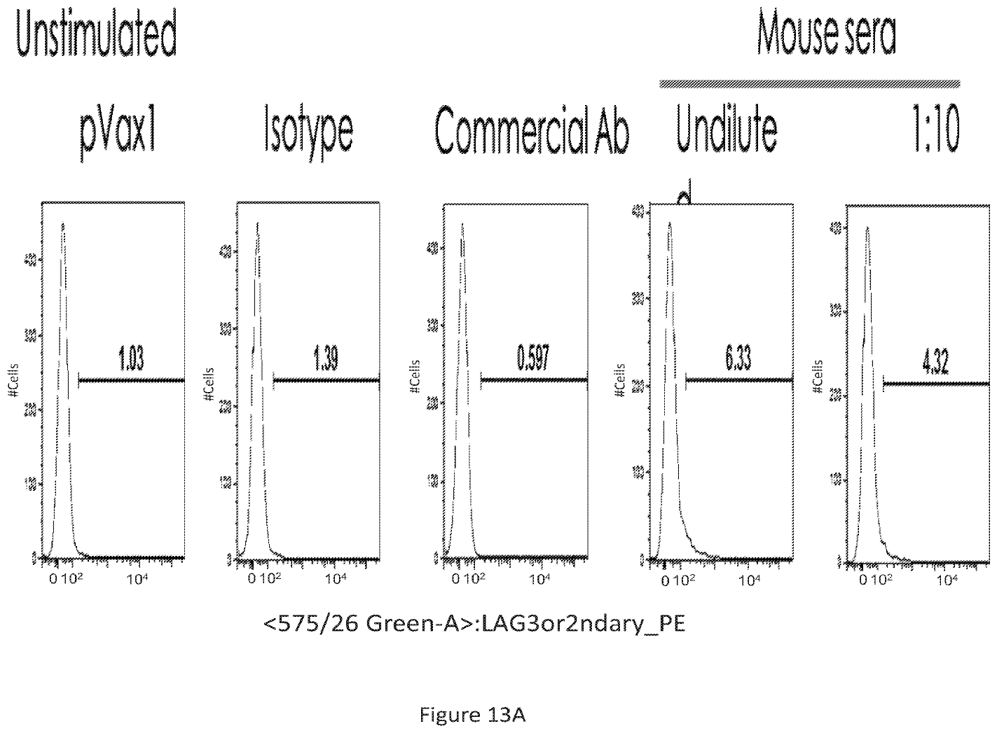

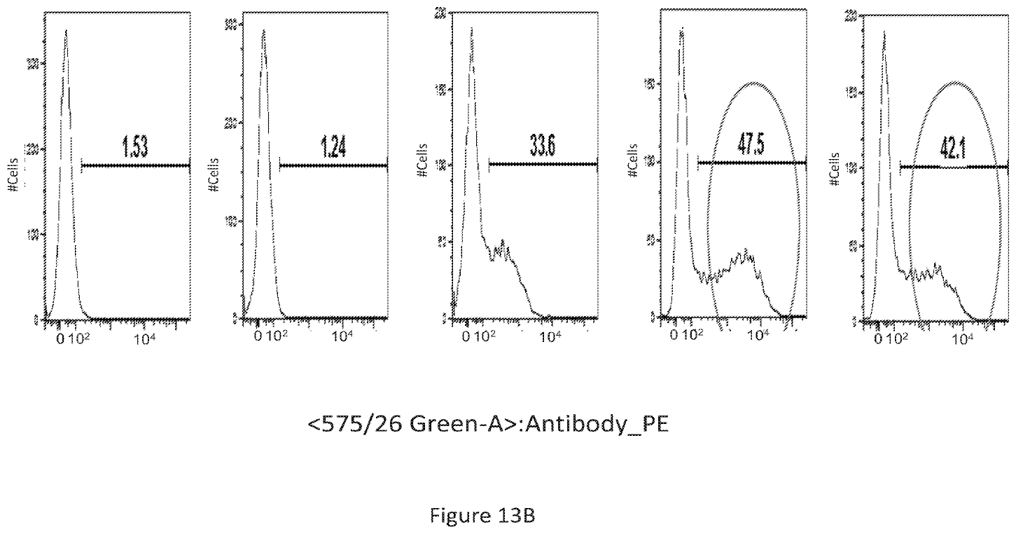

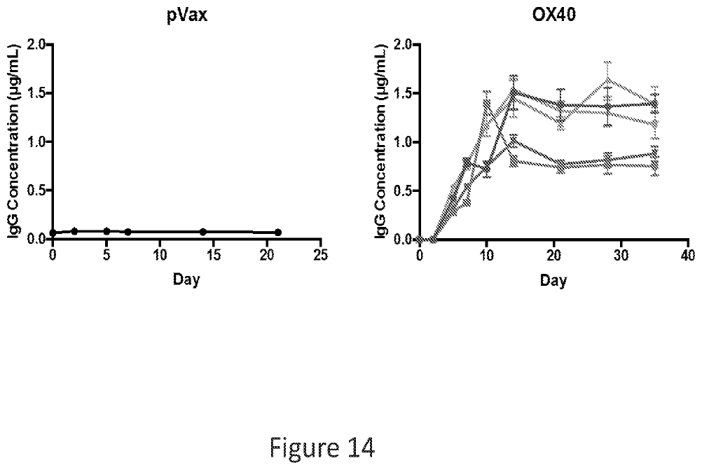

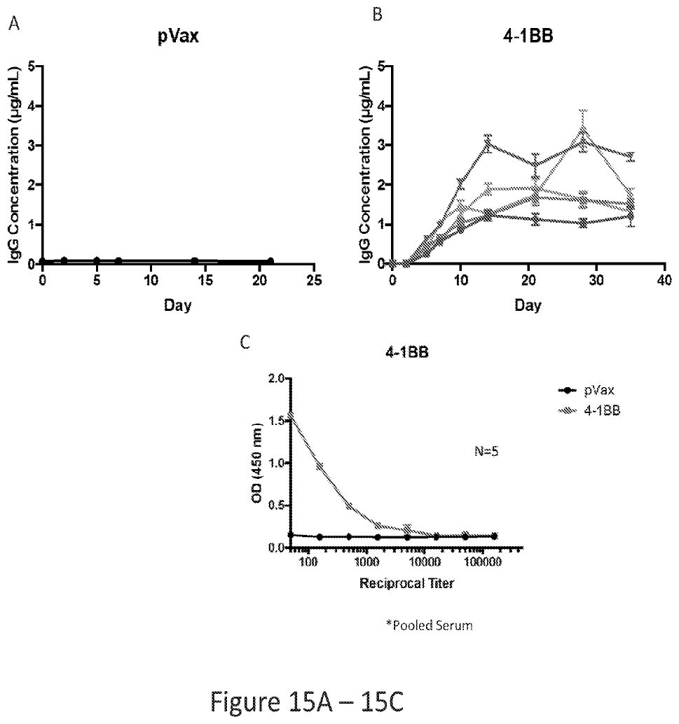

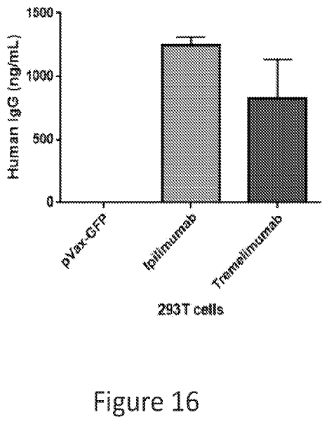

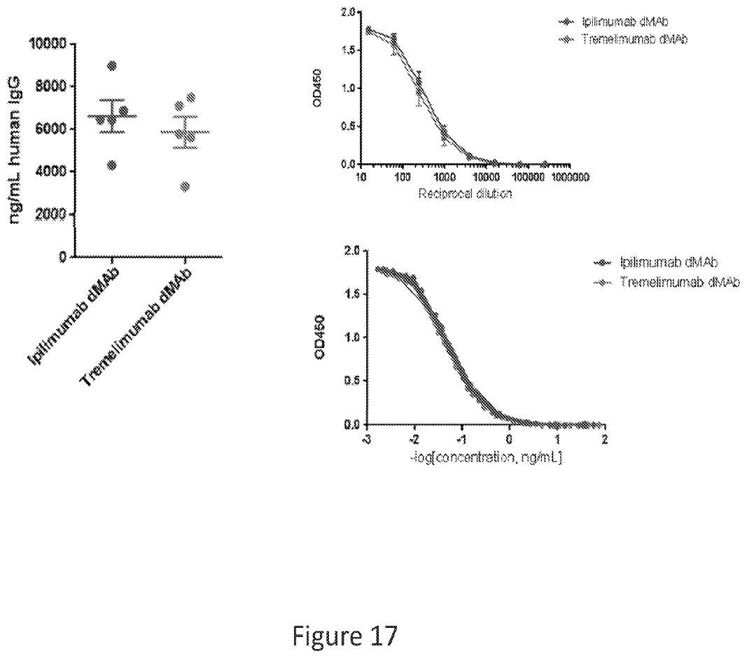

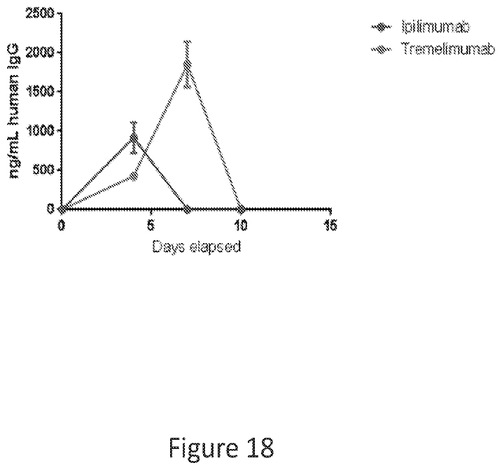

### DETAILED DESCRIPTION OF PREFERRED EMBODIMENTS

The present invention relates to a composition that can be used to increase or enhance an immune response, i.e., create a more effective immune response, by combining a vaccine, in many cases a synthetic antigen, with a checkpoint inhibitor, in particular, PD-1, PD-L1, LAG-3, GITR, CD40, OX40, CTLA-4, TIM-3, and 4-1BB antibodies (e.g., engineered MAb in the form of synthetic DNA plasmids).

Accordingly, with respect to engineered MAb in the form of synthetic DNA plasmids, the present invention relates to compositions comprising a recombinant nucleic acid sequence encoding an antibody, a fragment thereof, a variant thereof, or a combination thereof. The composition can be administered to a subject in need thereof to facilitate in vivo expression and formation of a synthetic antibody. In one embodiment the nucleotide sequence comprises nucleotide sequence described herein. For example, in one embodiment, the nucleotide sequence comprises a sequence of SEQ ID NOs: 1, 3, 5, 7, 9, 11, 13, 15, 17, 19, 21, 23, 25, 27, or a variant thereof or a fragment thereof. In another embodiment, the nucleotide sequence comprises sequence encoding the polypeptide sequence of SEQ ID NOs: 2, 4, 6, 8, 10, 12, 14, 16, 18, 20, 22, 24, 26, 28, or a variant thereof or a fragment thereof. In one embodiment the nucleotide sequence comprises an RNA sequence transcribed from a DNA sequence described herein. For example, in one embodiment, the nucleotide sequence comprises an RNA sequence transcribed by the DNA sequence of SEQ ID NOs: 1, 3, 5, 7, 9, 11, 13, 15, 17, 19, 21, 23, 25, 27, or a variant thereof or a fragment thereof. In another embodiment, the nucleotide sequence comprises an RNA sequence transcribed by a DNA sequence encoding the polypeptide sequence of SEQ ID NOs: 2, 4, 6, 8, 10, 12, 14, 16, 18, 20, 22, 24, 26, 28, or a variant thereof or a fragment thereof.

In one embodiment the nucleotide sequence encodes an amino acid sequence having at least about 80%, at least about 85%, at least about 90%, or at least about 95% identity over the entire length of the amino acid sequence to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, and SEQ ID NO:28. In one embodiment the nucleotide sequence encodes a fragment of an amino acid sequence having at least about 80%, at least about 85%, at least about 90%, or at least about 95% identity over the entire length of the amino acid sequence to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, and SEQ ID NO:28.

In one embodiment the nucleotide sequence has at least about 80%, at least about 85%, at least about 90%, or at least about 95% identity over the entire length of the nucleotide sequence to a nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, and SEQ ID NO:27. In one embodiment the nucleotide sequence is a fragment of a nucleotide sequence that has at least about 80%, at least about 85%, at least about 90%, or at least about 95% identity over the entire length of the nucleotide sequence to a nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO: 17, SEQ ID NO:19, SEQ ID NO:21, SEQ ID NO:23, SEQ ID NO:25, and SEQ ID NO: 27.

In particular, the heavy chain and light chain polypeptides expressed from the recombinant nucleic acid sequences can assemble into the synthetic antibody. The heavy chain polypeptide and the light chain polypeptide can interact with one another such that assembly results in the synthetic antibody being capable of binding the desired target (e.g., immune checkpoint molecule; PD-1, PD-L1, LAG-3, GITR, CD40, OX40, CTLA-4, TIM-3, 4-1BB, and the likes), being more immunogenic as compared to an antibody not assembled as described herein, and being capable of eliciting or inducing an immune response against the desired target.

Additionally, these synthetic antibodies are generated more rapidly in the subject than antibodies that are produced in response to antigen induced immune response. The synthetic antibodies are able to effectively bind and neutralize a range of targets. The synthetic antibodies are also able to effectively protect against and/or promote survival of disease.

In some instances, the antibodies of the invention can be administered in combination with the desired antigen; whereas, in other instances, the antibodies, can be administered separately from the antigen of the vaccine. In some instances the antibodies of the invention comprise a DNA sequence that encodes such antibody, which includes at least the variable regions of the immunoglobulin.

The composition of the present invention can increase the immune response to the antigen in the subject by increasing the CD8+ T cell response as compared to the vaccine not including checkpoint inhibitors. This increased CD8+ T cell response has cytolytic activity and secretes the anti-viral cytokine interferon-gamma (IFN-γ).

Aspects of the present invention include compositions for enhancing an immune response against an antigen in a subject in need thereof, comprising synthetic antibody in combination with a synthetic antigen capable of generating an immune response in the subject, or a biologically functional fragment or variant thereof.

The synthetic antigen can be an isolated DNA that encodes for the antigen. In one embodiment, the antigen is a tumor associated surface antigen. Illustrative examples of a tumor associated surface antigen are CD10, CD19, CD20, CD22, CD33, Fms-like tyrosine kinase 3 (FLT-3, CD135), chondroitin sulfate proteoglycan 4 (CSPG4, melanoma-associated chondroitin sulfate proteoglycan), Epidermal growth factor receptor (EGFR), Her2neu, Her3, IGFR, CD133, IL3R, fibroblast activating protein (FAP), CDCPI, Derlini, Tenascin, frizzled 1-10, the vascular antigens VEGFR2 (KDR/FLKI), VEGFR3 (FLT4, CD309), PDGFR-.alpha. (CD140a), PDGFR-.beta. (CD140b) Endoglin, CLEC14, Temi-8, and Tie2. Further examples may include A33, CAMPATH-1 (CDw52), Carcinoembryonic antigen (CEA), Carboanhydrase IX (MN/CA IX), CD21, CD25, CD30, CD34, CD37, CD44v6, CD45, CD133, de2-7 EGFR, EGFRvIII, EpCAM, Ep-CAM, Folate-binding protein, G250, Fms-like tyrosine kinase 3 (FLT-3, CD135), c-Kit (CD117), CSF1R (CD115), HLA-DR, IGFR, IL-2 receptor, IL3R, MCSP (Melanoma-associated cell surface chondroitin sulphate proteoglycane), Muc-1, Prostate-specific membrane antigen (PSMA), Prostate stem cell antigen (PSCA), Prostate specific antigen (PSA), and TAG-72. Examples of antigens expressed on the extracellular matrix of tumors are tenascin and the fibroblast activating protein (FAP).

In one embodiment, the synthetic antigen can be selected from the group consisting of: hTERT, PSA, PSMA, STEAP, PSCA, and PAP, WTT, tyrosinase, NYESO1, PRAME, MAGE, CMV, herpes, HIV, HPV, HCV, HBV, influenza, RSV, Plasmodium falciparum, and C. difficile.

The compositions provided herein can also include a pharmaceutically acceptable excipient.

Aspects of the invention also include methods for increasing an immune response in a subject in need thereof by administering any of the compositions provided herein to the subject. The methods of increasing an immune response can also include an electroporating step.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

“Antibody” may mean an antibody of classes IgG, IgM, IgA, IgD or IgE, or fragments, fragments or derivatives thereof, including Fab, F(ab′)2, Fd, and single chain antibodies, and derivatives thereof. The antibody may be an antibody isolated from the serum sample of mammal, a polyclonal antibody, affinity purified antibody, or mixtures thereof which exhibits sufficient binding specificity to a desired epitope or a sequence derived therefrom.

“Antibody fragment” or “fragment of an antibody” as used interchangeably herein refers to a portion of an intact antibody comprising the antigen-binding site or variable region. The portion does not include the constant heavy chain domains (i.e. CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)2 fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

“Adjuvant” as used herein means any molecule added to the vaccine described herein to enhance the immunogenicity of the antigens, and in particular refers to checkpoint inhibitor antibodies.

“Checkpoint inhibitor” as used herein means inhibitors or molecules that block immune checkpoints as commonly understood in the field of cancer immunotherapy. More commonly the checkpoint inhibitors are antibodies that block these immune checkpoints.

“Coding sequence” or “encoding nucleic acid” as used herein means the nucleic acid (RNA or DNA molecule) that comprise a nucleotide sequence which encodes a protein, such as an antibody, as set forth herein. The coding sequence may also comprise a DNA sequence which encodes an RNA sequence. The coding sequence can further include initiation and termination signals operably linked to regulatory elements including a promoter and polyadenylation signal capable of directing expression in the cells of an individual or mammal to which the nucleic acid is administered.

“Complement” or “complementary” as used herein means a nucleic acid can mean Watson-Crick (e.g., A-T/U and C-G) or Hoogsteen base pairing between nucleotides or nucleotide analogs of nucleic acid molecules.

“Electroporation,” “electro-permeabilization,” or “electro-kinetic enhancement” (“EP”) as used interchangeably herein means the use of a transmembrane electric field pulse to induce microscopic pathways (pores) in a bio-membrane; their presence allows biomolecules such as plasmids, oligonucleotides, siRNA, drugs, ions, and water to pass from one side of the cellular membrane to the other.

“Endogenous antibody” as used herein may refer to an antibody that is generated in a subject that is administered an effective dose of an antigen for induction of a humoral immune response.

“Fragment” as used herein means a nucleic acid sequence or a portion thereof that encodes a polypeptide capable of eliciting an immune response in a mammal. The fragments can be DNA fragments selected from at least one of the various nucleotide sequences that encode protein fragments set forth below. Fragments can comprise at least 10%, at least 20%, at least 30%, at least 40%, at least 50%, at least 60%, at least 70%, at least 80%, at least 90%, or at least 95% of one or more of the nucleic acid sequences set forth below. In some embodiments, fragments can comprise at least 20 nucleotides or more, at least 30 nucleotides or more, at least 40 nucleotides or more, at least 50 nucleotides or more, at least 60 nucleotides or more, at least 70 nucleotides or more, at least 80 nucleotides or more, at least 90 nucleotides or more, at least 100 nucleotides or more, at least 150 nucleotides or more, at least 200 nucleotides or more, at least 250 nucleotides or more, at least 300 nucleotides or more, at least 350 nucleotides or more, at least 400 nucleotides or more, at least 450 nucleotides or more, at least 500 nucleotides or more, at least 550 nucleotides or more, at least 600 nucleotides or more, at least 650 nucleotides or more, at least 700 nucleotides or more, at least 750 nucleotides or more, at least 800 nucleotides or more, at least 850 nucleotides or more, at least 900 nucleotides or more, at least 950 nucleotides or more, or at least 1000 nucleotides or more of at least one of the nucleic acid sequences set forth below.

Fragment as used herein also means a polypeptide sequence or a portion thereof that is capable of eliciting an immune response in a mammal. The fragments can be polypeptide fragments selected from at least one of the various amino acid sequence set forth below. Fragments can comprise at least 10%, at least 20%, at least 30%, at least 40%, at least 50%, at least 60%, at least 70%, at least 80%, at least 90%, or at least 95% of one or more of the proteins set forth below. In some embodiments, fragments can comprise at least 20 amino acids or more, at least 30 amino acids or more, at least 40 amino acids or more, at least 50 amino acids or more, at least 60 amino acids or more, at least 70 amino acids or more, at least 80 amino acids or more, at least 90 amino acids or more, at least 100 amino acids or more, at least 110 amino acids or more, at least 120 amino acids or more, at least 130 amino acids or more, at least 140 amino acids or more, at least 150 amino acids or more, at least 160 amino acids or more, at least 170 amino acids or more, at least 180 amino acids or more, at least 190 amino acids or more, at least 200 amino acids or more, at least 210 amino acids or more, at least 220 amino acids or more, at least 230 amino acids or more, or at least 240 amino acids or more of at least one of the proteins set forth below.

“Genetic construct” as used herein refers to the DNA or RNA molecules that comprise a nucleotide sequence which encodes a protein, such as an antibody. The genetic construct may also refer to a DNA molecule which transcribes an RNA. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

“Identical” or “identity” as used herein in the context of two or more nucleic acids or polypeptide sequences, means that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

“Immune response” as used herein means the activation of a host's immune system, e.g., that of a mammal, in response to the introduction of antigen. The immune response can be in the form of a cellular or humoral response, or both.

“Nucleic acid” or “oligonucleotide” or “polynucleotide” as used herein means at least two nucleotides covalently linked together. The depiction of a single strand also defines the sequence of the complementary strand. Thus, a nucleic acid also encompasses the complementary strand of a depicted single strand. Many variants of a nucleic acid can be used for the same purpose as a given nucleic acid. Thus, a nucleic acid also encompasses substantially identical nucleic acids and complements thereof. A single strand provides a probe that can hybridize to a target sequence under stringent hybridization conditions. Thus, a nucleic acid also encompasses a probe that hybridizes under stringent hybridization conditions.

Nucleic acids can be single stranded or double stranded, or can contain portions of both double stranded and single stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods.

“Operably linked” as used herein means that expression of a gene is under the control of a promoter with which it is spatially connected. A promoter can be positioned 5′ (upstream) or 3′ (downstream) of a gene under its control. The distance between the promoter and a gene can be approximately the same as the distance between that promoter and the gene it controls in the gene from which the promoter is derived. As is known in the art, variation in this distance can be accommodated without loss of promoter function.

A “peptide,” “protein,” or “polypeptide” as used herein can mean a linked sequence of amino acids and can be natural, synthetic, or a modification or combination of natural and synthetic.

“Promoter” as used herein means a synthetic or naturally-derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter.

“Signal peptide” and “leader sequence” are used interchangeably herein and refer to an amino acid sequence that can be linked at the amino terminus of a synthetic antigen, including some of the examples cited herein. Signal peptides/leader sequences typically direct localization of a protein. Signal peptides/leader sequences used herein preferably facilitate secretion of the protein from the cell in which it is produced. Signal peptides/leader sequences are often cleaved from the remainder of the protein, often referred to as the mature protein, upon secretion from the cell. Signal peptides/leader sequences are linked at the N terminus of the protein.

“Stringent hybridization conditions” as used herein may mean conditions under which a first nucleic acid sequence (e.g., probe) will hybridize to a second nucleic acid sequence (e.g., target), such as in a complex mixture of nucleic acids. Stringent conditions are sequence dependent and will be different in different circumstances. Stringent conditions may be selected to be about 5-10° C. lower than the thermal melting point (Tₘ) for the specific sequence at a defined ionic strength pH. The Tₘ may be the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.

“Subject” as used herein can mean a mammal that wants to or is in need of being immunized with the herein described vaccine. The mammal can be a human, chimpanzee, dog, cat, horse, cow, pig, chicken mouse, or rat.

“Substantially complementary” as used herein may mean that a first sequence is at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or that the two sequences hybridize under stringent hybridization conditions.

“Substantially identical” as used herein can mean that a first and second amino acid sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more amino acids. Substantially identical can also mean that a first nucleic acid sequence and a second nucleic acid sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more nucleotides.

“Synthetic antibody” as used herein refers to an antibody that is encoded by the recombinant nucleic acid sequence.

“Treatment” or “treating,” as used herein can mean protecting of an animal from a disease through means of preventing, suppressing, repressing, or completely eliminating the disease. Preventing the disease involves administering a vaccine of the present invention to an animal prior to onset of the disease. Suppressing the disease involves administering a vaccine of the present invention to an animal after induction of the disease but before its clinical appearance. Repressing the disease involves administering a vaccine of the present invention to an animal after clinical appearance of the disease.

“Variant” used herein with respect to a nucleic acid means (i) a portion or fragment of a referenced nucleotide sequence; (ii) the complement of a referenced nucleotide sequence or portion thereof; (iii) a nucleic acid that is substantially identical to a referenced nucleic acid or the complement thereof, or (iv) a nucleic acid that hybridizes under stringent conditions to the referenced nucleic acid, complement thereof, or a sequences substantially identical thereto.

Variant can further be defined as a peptide or polypeptide that differs in amino acid sequence by the insertion, deletion, or conservative substitution of amino acids, but retain at least one biological activity. Representative examples of “biological activity” include the ability to be bound by a specific antibody or to promote an immune response. Variant can also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of 2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. Substitution of amino acids having similar hydrophilicity values can result in peptides retaining biological activity, for example immunogenicity, as is understood in the art. Substitutions can be performed with amino acids having hydrophilicity values within +2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant may be a nucleic acid sequence that is substantially identical over the full length of the full gene sequence or a fragment thereof. The nucleic acid sequence may be 80%, 81%, 82%, 83%, 84%, 85%, 86%, 87%8, 88%, 89%, 90%, 91%, 92%, 93%, 94%, 95%, 96%, 97%, 98%, 99%, or 100% identical over the full length of the gene sequence or a fragment thereof. A variant may be an amino acid sequence that is substantially identical over the full length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof

“Vector” as used herein means a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector, and preferably, is a DNA plasmid.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. COMPOSITIONS

Provided herein are compositions comprising an antigen and checkpoint inhibitors, preferably checkpoint inhibitor antibodies. The antibodies preferably are synthetic antibodies. The synthetic antibodies preferably are PD-1 antibody, PD-L1 antibody, LAG-3 antibody, GITR antibody, CD40 antibody, OX40 antibody, CTLA-4 antibody, TIM-3 antibody, and/or 4-1BB antibody. The invention also includes novel sequences for use for producing antibodies in mammalian cells or for delivery in DNA or RNA vectors including bacterial, yeast, as well as viral vectors.

The present invention relates to a composition comprising a recombinant nucleic acid sequence encoding an antibody, a fragment thereof, a variant thereof, or a combination thereof. The composition, when administered to a subject in need thereof, can result in the generation of a synthetic antibody in the subject. The synthetic antibody can bind a target molecule (i.e., PD-1, PD-L1, LAG-3, GITR, CD40, OX40, CTLA-4, TIM-3, and/or 4-1BB) present in the subject. Such binding can neutralize the target, block recognition of the target by another molecule, for example, a protein or nucleic acid, and elicit or induce an immune response to the target.

In one embodiment, the composition comprises a nucleotide sequence encoding a synthetic antibody. In one embodiment, the composition comprises a nucleic acid molecule comprising a first nucleotide sequence encoding a first synthetic antibody and a second nucleotide sequence encoding a second synthetic antibody. In one embodiment, the nucleic acid molecule comprises a nucleotide sequence encoding a cleavage domain.

In one embodiment, the nucleic acid molecule comprises a nucleotide sequence encoding anti-PD-1 antibody, anti-PD-L1 antibody, anti-LAG-3 antibody, anti-GITR antibody, anti-CD40 antibody, anti-OX40 antibody, anti-CTLA-4, anti-TIM-3 antibody, and/or anti-4-1BB antibody. In one embodiment, the nucleotide sequence encoding the antibody comprises codon optimized nucleic acid sequences encoding the variable VH and VL regions of the antibody. In one embodiment, the nucleotide sequence encoding the antibody comprises codon optimized nucleic acid sequences encoding CH and CL regions of human IgG1κ.

In one embodiment, the first nucleotide sequence encoding a first synthetic antibody comprises a first domain encoding the heavy chain region and a second domain encoding the light chain region of the first synthetic antibody. In one embodiment, the second nucleotide sequence encoding a second synthetic antibody comprises a first domain encoding the heavy chain region and a second domain encoding the light chain region of the second synthetic antibody. In one embodiment, the nucleic acid molecule comprises at least one nucleotide sequence encoding a first domain encoding the heavy chain region and a second domain encoding the light chain region of an antibody selected from the group anti-PD-1 antibody, anti-PD-L1 antibody, anti-LAG-3 antibody, anti-GITR antibody, anti-CD40 antibody, anti-OX40 antibody, anti-CTLA-4 antibody, anti-TIM-3 antibody, and anti-4-1BB antibody.

In one embodiment, the combination can be a single formulation or can be separate and administered in sequence (either antigen first and then checkpoint inhibitor, or checkpoint inhibitor first and then antigen). The composition can increase antigen presentation and the overall immune response to the antigen in a subject. The combination of antigen and checkpoint inhibitor induces the immune system more efficiently than a composition comprising the antigen alone. This more efficient immune response provides increased efficacy in the treatment and/or prevention of any disease, in particular cancer, pathogen, or virus.

The antigen and checkpoint inhibitors, preferably are anti-PD-1 antibody, anti-PD-L1 antibody, anti-LAG-3 antibody, anti-GITR antibody, anti-CD40 antibody, anti-OX40 antibody, anti-CTLA-4 antibody, anti-TIM-3 antibody, and/or anti-4-1BB antibody, of the composition can be administered together or separately to the subject in need thereof. In some instances, the checkpoint inhibitors can be administered separately from the antigen of the composition.

In some embodiments, the checkpoint inhibitors can be administered at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 48 hours, 60 hours, 72 hours, 84 hours, or 96 hours before or after administration of the antigen to the subject. In other embodiments, the PD1 antibody or PDL1 antibody can be administered at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or 90 days before or after administration of the antigen to the subject.

In still other embodiments, the checkpoint inhibitors can be administered at least 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, or 15 weeks before or after administration of the antigen to the subject. In other embodiments, the antibody or antibodies can be administered about 12 hours to about 15 weeks, about 12 hours to about 10 weeks, about 12 hours to about weeks, about 12 hours to about 1 week, about 12 hours to about 60 hours, about 12 hours to about 48 hours, about 24 hours to about 15 weeks, about 60 hours to about 15 weeks, about 96 hours to about 15 weeks, about 1 day to about 15 weeks, about 5 days to about 15 weeks, about 10 days to about 15 weeks, about 15 days to about 15 weeks, about 20 days to about 15 weeks, about 25 days to about 15 weeks, about 30 days to about 15 weeks, about 1 week to about 15 weeks, about 5 weeks to about 15 weeks, or about 10 weeks to about 15 weeks before or after administration of the antigen to the subject.

The composition of the present invention can have features required of effective compositions such as being safe so the composition itself does not cause illness or death; being protective against illness resulting from exposure to live pathogens such as viruses or bacteria; inducing neutralizing antibody to prevent infection of cells; inducing protective T cell against intracellular pathogens; and providing ease of administration, few side effects, biological stability, and low cost per dose. The composition can accomplish some or all of these features by combining the antigen with the checkpoint inhibitors, preferably the anti-PD-1 antibody, anti-PD-L1 antibody, anti-LAG-3 antibody, anti-GITR antibody, anti-CD40 antibody, anti-OX40 antibody, anti-CTLA-4 antibody, anti-TIM-3 antibody, and/or anti-4-1BB antibody as discussed below.

The composition can further modify epitope presentation within the antigen to induce greater immune response to the antigen that a composition comprising the antigen alone. The composition can further induce an immune response when administered to different tissues such as the muscle or the skin.

a. Checkpoint Inhibitors

Checkpoint inhibitors can be any antagonist to the various immune checkpoints, and are preferably antibodies that block immune checkpoints. The antibodies can be a protein including a Fab, monoclonal or polyclonal. The antibodies can also be a DNA expression construct that encodes for and can express functional antibodies. The vaccine can further comprise a PD-1 antibody, PD-L1 antibody, LAG-3 antibody, GITR antibody, CD40 antibody, OX40 antibody, CTLA-4 antibody, TIM-3 antibody, and/or a 4-1BB antibody. The antibody can be a synthetic antibody comprised of DNA sequence encoding at least the variable regions of an immunoglobulin. Such antibody can be generated by identifying or screening for the antibody described above, which is reactive to or binds the antigen described above. The method of identifying or screening for the antibody can use the antigen in methodologies known to those skilled in art to identify or screen for the antibody. Such methodologies can include, but are not limited to, selection of the antibody from a library (e.g., phage display) and immunization of an animal followed by isolation and/or purification of the antibody. See for example methods available in Rajan, S., and Sidhu, S., Methods in Enzymology, vol 502, Chapter One “Simplified Synthetic Antibody Libraries (2012), which is incorporated herein in its entirety.

Any antibodies of the invention can also be combined with other checkpoint inhibitor antibodies, including anti-CTLA-4, and others. The checkpoint inhibitors can be a known product such as, for example, ipilimumab, tremelimumab, nivolumab, pembrolizumab, pidilizumab, BMS-936559 (See ClinicalTrials.gov Identifier NCT02028403), MPDL3280A (Roche, see ClinicalTrials.gov Identifier NCT02008227), MDX1105-01 (Bristol Myers Squibb, see ClinicalTrials.gov Identifier NCT00729664), MED14736 (MedImmune, See ClinicalTrials.gov Identifier NCT01693562), and MK-3475 (Merck, see ClinicalTrials.gov Identifier NCT02129556).

b. Recombinant Nucleic Acid Sequence Construct

The recombinant nucleic acid sequence can include one or more recombinant nucleic acid sequence constructs. The recombinant nucleic acid sequence construct can include one or more components, which are described in more detail below.

The recombinant nucleic acid sequence construct can include a heterologous nucleic acid sequence that encodes a heavy chain polypeptide, a fragment thereof, a variant thereof, or a combination thereof. The recombinant nucleic acid sequence construct can include a heterologous nucleic acid sequence that encodes a light chain polypeptide, a fragment thereof, a variant thereof, or a combination thereof. The recombinant nucleic acid sequence construct can also include a heterologous nucleic acid sequence that encodes a protease or peptidase cleavage site. The recombinant nucleic acid sequence construct can include one or more leader sequences, in which each leader sequence encodes a signal peptide. The recombinant nucleic acid sequence construct can include one or more promoters, one or more introns, one or more transcription termination regions, one or more initiation codons, one or more termination or stop codons, and/or one or more polyadenylation signals. The recombinant nucleic acid sequence construct can also include one or more linker or tag sequences. The tag sequence can encode a hemagglutinin (HA) tag.

### (1) Heavy Chain Polypeptide

The recombinant nucleic acid sequence construct can include the heterologous nucleic acid encoding the heavy chain polypeptide, a fragment thereof, a variant thereof, or a combination thereof. The heavy chain polypeptide can include a variable heavy chain (VH) region and/or at least one constant heavy chain (CH) region. The at least one constant heavy chain region can include a constant heavy chain region 1 (CH1), a constant heavy chain region 2 (CH2), and a constant heavy chain region 3 (CH3), and/or a hinge region.

In some embodiments, the heavy chain polypeptide can include a VH region and a CH1 region. In other embodiments, the heavy chain polypeptide can include a VH region, a CH1 region, a hinge region, a CH2 region, and a CH3 region.

The heavy chain polypeptide can include a complementarity determining region (“CDR”) set. The CDR set can contain three hypervariable regions of the VH region. Proceeding from N-terminus of the heavy chain polypeptide, these CDRs are denoted “CDR1,” “CDR2,” and “CDR3,” respectively. CDR1, CDR2, and CDR3 of the heavy chain polypeptide can contribute to binding or recognition of the antigen.

### (2) Light Chain Polypeptide

The recombinant nucleic acid sequence construct can include the heterologous nucleic acid sequence encoding the light chain polypeptide, a fragment thereof, a variant thereof, or a combination thereof. The light chain polypeptide can include a variable light chain (VL) region and/or a constant light chain (CL) region.

The light chain polypeptide can include a complementarity determining region (“CDR”) set. The CDR set can contain three hypervariable regions of the VL region. Proceeding from N-terminus of the light chain polypeptide, these CDRs are denoted “CDR1,” “CDR2,” and “CDR3,” respectively. CDR1, CDR2, and CDR3 of the light chain polypeptide can contribute to binding or recognition of the antigen.

### (3) Protease Cleavage Site

The recombinant nucleic acid sequence construct can include the heterologous nucleic acid sequence encoding the protease cleavage site. The protease cleavage site can be recognized by a protease or peptidase. The protease can be an endopeptidase or endoprotease, for example, but not limited to, furin, elastase, HtrA, calpain, trypsin, chymotrypsin, trypsin, and pepsin. The protease can be furin. In other embodiments, the protease can be a serine protease, a threonine protease, cysteine protease, aspartate protease, metalloprotease, glutamic acid protease, or any protease that cleaves an internal peptide bond (i.e., does not cleave the N-terminal or C-terminal peptide bond).

The protease cleavage site can include one or more amino acid sequences that promote or increase the efficiency of cleavage. The one or more amino acid sequences can promote or increase the efficiency of forming or generating discrete polypeptides. The one or more amino acids sequences can include a 2A peptide sequence.

### (4) Linker Sequence

The recombinant nucleic acid sequence construct can include one or more linker sequences. The linker sequence can spatially separate or link the one or more components described herein. In other embodiments, the linker sequence can encode an amino acid sequence that spatially separates or links two or more polypeptides.

### (5) Promoter

The recombinant nucleic acid sequence construct can include one or more promoters. The one or more promoters may be any promoter that is capable of driving gene expression and regulating gene expression. Such a promoter is a cis-acting sequence element required for transcription via a DNA dependent RNA polymerase. Selection of the promoter used to direct gene expression depends on the particular application. The promoter may be positioned about the same distance from the transcription start in the recombinant nucleic acid sequence construct as it is from the transcription start site in its natural setting. However, variation in this distance may be accommodated without loss of promoter function.

The promoter may be operably linked to the heterologous nucleic acid sequence encoding the heavy chain polypeptide and/or light chain polypeptide. The promoter may be a promoter shown effective for expression in eukaryotic cells. The promoter operably linked to the coding sequence may be a CMV promoter, a promoter from simian virus 40 (SV40), such as SV40 early promoter and SV40 later promoter, a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human actin, human myosin, human hemoglobin, human muscle creatine, human polyhedrin, or human metalothionein.

The promoter can be a constitutive promoter or an inducible promoter, which initiates transcription only when the host cell is exposed to some particular external stimulus. In the case of a multicellular organism, the promoter can also be specific to a particular tissue or organ or stage of development. The promoter may also be a tissue specific promoter, such as a muscle or skin specific promoter, natural or synthetic. Examples of such promoters are described in US patent application publication no. US20040175727, the contents of which are incorporated herein in its entirety.

The promoter can be associated with an enhancer. The enhancer can be located upstream of the coding sequence. The enhancer may be human actin, human myosin, human hemoglobin, human muscle creatine or a viral enhancer such as one from CMV, FMDV, RSV or EBV. Polynucleotide function enhances are described in U.S. Pat. Nos. 5,593,972, 5,962,428, and WO94/016737, the contents of each are fully incorporated by reference.

### (6) Intron

The recombinant nucleic acid sequence construct can include one or more introns. Each intron can include functional splice donor and acceptor sites. The intron can include an enhancer of splicing. The intron can include one or more signals required for efficient splicing.

### (7) Transcription Termination Region

The recombinant nucleic acid sequence construct can include one or more transcription termination regions. The transcription termination region can be downstream of the coding sequence to provide for efficient termination. The transcription termination region can be obtained from the same gene as the promoter described above or can be obtained from one or more different genes.

### (8) Initiation Codon

The recombinant nucleic acid sequence construct can include one or more initiation codons. The initiation codon can be located upstream of the coding sequence. The initiation codon can be in frame with the coding sequence. The initiation codon can be associated with one or more signals required for efficient translation initiation, for example, but not limited to, a ribosome binding site.

### (9) Termination Codon

The recombinant nucleic acid sequence construct can include one or more termination or stop codons. The termination codon can be downstream of the coding sequence. The termination codon can be in frame with the coding sequence. The termination codon can be associated with one or more signals required for efficient translation termination.

### (10) Polyadenylation Signal

The recombinant nucleic acid sequence construct can include one or more polyadenylation signals. The polyadenylation signal can include one or more signals required for efficient polyadenylation of the transcript. The polyadenylation signal can be positioned downstream of the coding sequence. The polyadenylation signal may be a SV40 polyadenylation signal, LTR polyadenylation signal, bovine growth hormone (bGH) polyadenylation signal, human growth hormone (hGH) polyadenylation signal, or human 3-globin polyadenylation signal. The SV40 polyadenylation signal may be a polyadenylation signal from a pCEP4 plasmid (Invitrogen, San Diego, CA).

### (11) Leader Sequence

The recombinant nucleic acid sequence construct can include one or more leader sequences. The leader sequence can encode a signal peptide. The signal peptide can be an immunoglobulin (Ig) signal peptide, for example, but not limited to, an IgG signal peptide and a IgE signal peptide.

c. Arrangement of the Recombinant Nucleic Acid Sequence Construct

As described above, the recombinant nucleic acid sequence can include one or more recombinant nucleic acid sequence constructs, in which each recombinant nucleic acid sequence construct can include one or more components. The one or more components are described in detail above. The one or more components, when included in the recombinant nucleic acid sequence construct, can be arranged in any order relative to one another. In some embodiments, the one or more components can be arranged in the recombinant nucleic acid sequence construct as described below.

### (1) Arrangement 1

In one arrangement, a first recombinant nucleic acid sequence construct can include the heterologous nucleic acid sequence encoding the heavy chain polypeptide and a second recombinant nucleic acid sequence construct can include the heterologous nucleic acid sequence encoding the light chain polypeptide.

The first recombinant nucleic acid sequence construct can be placed in a vector. The second recombinant nucleic acid sequence construct can be placed in a second or separate vector. Placement of the recombinant nucleic acid sequence construct into the vector is described in more detail below.

The first recombinant nucleic acid sequence construct can also include the promoter, intron, transcription termination region, initiation codon, termination codon, and/or polyadenylation signal. The first recombinant nucleic acid sequence construct can further include the leader sequence, in which the leader sequence is located upstream (or 5′) of the heterologous nucleic acid sequence encoding the heavy chain polypeptide. Accordingly, the signal peptide encoded by the leader sequence can be linked by a peptide bond to the heavy chain polypeptide.

The second recombinant nucleic acid sequence construct can also include the promoter, initiation codon, termination codon, and polyadenylation signal. The second recombinant nucleic acid sequence construct can further include the leader sequence, in which the leader sequence is located upstream (or 5′) of the heterologous nucleic acid sequence encoding the light chain polypeptide. Accordingly, the signal peptide encoded by the leader sequence can be linked by a peptide bond to the light chain polypeptide.

Accordingly, one example of arrangement 1 can include the first vector (and thus first recombinant nucleic acid sequence construct) encoding the heavy chain polypeptide that includes VH and CH1, and the second vector (and thus second recombinant nucleic acid sequence construct) encoding the light chain polypeptide that includes VL and CL. A second example of arrangement 1 can include the first vector (and thus first recombinant nucleic acid sequence construct) encoding the heavy chain polypeptide that includes VH, CH1, hinge region, CH2, and CH3, and the second vector (and thus second recombinant nucleic acid sequence construct) encoding the light chain polypeptide that includes VL and CL.

### (2) Arrangement 2

In a second arrangement, the recombinant nucleic acid sequence construct can include the heterologous nucleic acid sequence encoding the heavy chain polypeptide and the heterologous nucleic acid sequence encoding the light chain polypeptide. The heterologous nucleic acid sequence encoding the heavy chain polypeptide can be positioned upstream (or 5′) of the heterologous nucleic acid sequence encoding the light chain polypeptide. Alternatively, the heterologous nucleic acid sequence encoding the light chain polypeptide can be positioned upstream (or 5′) of the heterologous nucleic acid sequence encoding the heavy chain polypeptide.

The recombinant nucleic acid sequence construct can be placed in the vector as described in more detail below.

The recombinant nucleic acid sequence construct can include the heterologous nucleic acid sequence encoding the protease cleavage site and/or the linker sequence. If included in the recombinant nucleic acid sequence construct, the heterologous nucleic acid sequence encoding the protease cleavage site can be positioned between the heterologous nucleic acid sequence encoding the heavy chain polypeptide and the heterologous nucleic acid sequence encoding the light chain polypeptide. Accordingly, the protease cleavage site allows for separation of the heavy chain polypeptide and the light chain polypeptide into distinct polypeptides upon expression. In other embodiments, if the linker sequence is included in the recombinant nucleic acid sequence construct, then the linker sequence can be positioned between the heterologous nucleic acid sequence encoding the heavy chain polypeptide and the heterologous nucleic acid sequence encoding the light chain polypeptide.

The recombinant nucleic acid sequence construct can also include the promoter, intron, transcription termination region, initiation codon, termination codon, and/or polyadenylation signal. The recombinant nucleic acid sequence construct can include one or more promoters. The recombinant nucleic acid sequence construct can include two promoters such that one promoter can be associated with the heterologous nucleic acid sequence encoding the heavy chain polypeptide and the second promoter can be associated with the heterologous nucleic acid sequence encoding the light chain polypeptide. In still other embodiments, the recombinant nucleic acid sequence construct can include one promoter that is associated with the heterologous nucleic acid sequence encoding the heavy chain polypeptide and the heterologous nucleic acid sequence encoding the light chain polypeptide.

The recombinant nucleic acid sequence construct can further include two leader sequences, in which a first leader sequence is located upstream (or 5′) of the heterologous nucleic acid sequence encoding the heavy chain polypeptide and a second leader sequence is located upstream (or 5′) of the heterologous nucleic acid sequence encoding the light chain polypeptide. Accordingly, a first signal peptide encoded by the first leader sequence can be linked by a peptide bond to the heavy chain polypeptide and a second signal peptide encoded by the second leader sequence can be linked by a peptide bond to the light chain polypeptide.

Accordingly, one example of arrangement 2 can include the vector (and thus recombinant nucleic acid sequence construct) encoding the heavy chain polypeptide that includes VH and CH1, and the light chain polypeptide that includes VL and CL, in which the linker sequence is positioned between the heterologous nucleic acid sequence encoding the heavy chain polypeptide and the heterologous nucleic acid sequence encoding the light chain polypeptide.

A second example of arrangement of 2 can include the vector (and thus recombinant nucleic acid sequence construct) encoding the heavy chain polypeptide that includes VH and CH1, and the light chain polypeptide that includes VL and CL, in which the heterologous nucleic acid sequence encoding the protease cleavage site is positioned between the heterologous nucleic acid sequence encoding the heavy chain polypeptide and the heterologous nucleic acid sequence encoding the light chain polypeptide.

A third example of arrangement 2 can include the vector (and thus recombinant nucleic acid sequence construct) encoding the heavy chain polypeptide that includes VH, CH1, hinge region, CH2, and CH3, and the light chain polypeptide that includes VL and CL, in which the linker sequence is positioned between the heterologous nucleic acid sequence encoding the heavy chain polypeptide and the heterologous nucleic acid sequence encoding the light chain polypeptide.

A fourth example of arrangement of 2 can include the vector (and thus recombinant nucleic acid sequence construct) encoding the heavy chain polypeptide that includes VH, CH1, hinge region, CH2, and CH3, and the light chain polypeptide that includes VL and CL, in which the heterologous nucleic acid sequence encoding the protease cleavage site is positioned between the heterologous nucleic acid sequence encoding the heavy chain polypeptide and the heterologous nucleic acid sequence encoding the light chain polypeptide.

d. Expression from the Recombinant Nucleic Acid Sequence Construct

As described above, the recombinant nucleic acid sequence construct can include, amongst the one or more components, the heterologous nucleic acid sequence encoding the heavy chain polypeptide and/or the heterologous nucleic acid sequence encoding the light chain polypeptide. Accordingly, the recombinant nucleic acid sequence construct can facilitate expression of the heavy chain polypeptide and/or the light chain polypeptide.

When arrangement 1 as described above is utilized, the first recombinant nucleic acid sequence construct can facilitate the expression of the heavy chain polypeptide and the second recombinant nucleic acid sequence construct can facilitate expression of the light chain polypeptide. When arrangement 2 as described above is utilized, the recombinant nucleic acid sequence construct can facilitate the expression of the heavy chain polypeptide and the light chain polypeptide.

Upon expression, for example, but not limited to, in a cell, organism, or mammal, the heavy chain polypeptide and the light chain polypeptide can assemble into the synthetic antibody. In particular, the heavy chain polypeptide and the light chain polypeptide can interact with one another such that assembly results in the synthetic antibody being capable of binding the antigen. In other embodiments, the heavy chain polypeptide and the light chain polypeptide can interact with one another such that assembly results in the synthetic antibody being more immunogenic as compared to an antibody not assembled as described herein. In still other embodiments, the heavy chain polypeptide and the light chain polypeptide can interact with one another such that assembly results in the synthetic antibody being capable of eliciting or inducing an immune response against the antigen.

e. Vectors

The recombinant nucleic acid sequence construct described above can be placed in one or more vectors. The one or more vectors can contain an origin of replication. The one or more vectors can be a plasmid, bacteriophage, bacterial artificial chromosome or yeast artificial chromosome. The one or more vectors can be either a self-replication extra chromosomal vector, or a vector which integrates into a host genome.

Vectors include, but are not limited to, plasmids, expression vectors, recombinant viruses, any form of recombinant “naked DNA” vector, and the like. A “vector” comprises a nucleic acid which can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and include both the expression and non-expression plasmids. In some embodiments, the vector includes linear DNA, enzymatic DNA or synthetic DNA. Where a recombinant microorganism or cell culture is described as hosting an “expression vector” this includes both extra-chromosomal circular and linear DNA and DNA that has been incorporated into the host chromosome(s). Where a vector is being maintained by a host cell, the vector may either be stably replicated by the cells during mitosis as an autonomous structure, or is incorporated within the host's genome.

The one or more vectors can be a heterologous expression construct, which is generally a plasmid that is used to introduce a specific gene into a target cell. Once the expression vector is inside the cell, the heavy chain polypeptide and/or light chain polypeptide that are encoded by the recombinant nucleic acid sequence construct is produced by the cellular-transcription and translation machinery ribosomal complexes. The one or more vectors can express large amounts of stable messenger RNA, and therefore proteins.

### (1) Expression Vector

The one or more vectors can be a circular plasmid or a linear nucleic acid. The circular plasmid and linear nucleic acid are capable of directing expression of a particular nucleotide sequence in an appropriate subject cell. The one or more vectors comprising the recombinant nucleic acid sequence construct may be chimeric, meaning that at least one of its components is heterologous with respect to at least one of its other components.

### (2) Plasmid

The one or more vectors can be a plasmid. The plasmid may be useful for transfecting cells with the recombinant nucleic acid sequence construct. The plasmid may be useful for introducing the recombinant nucleic acid sequence construct into the subject. The plasmid may also comprise a regulatory sequence, which may be well suited for gene expression in a cell into which the plasmid is administered.

The plasmid may also comprise a mammalian origin of replication in order to maintain the plasmid extrachromosomally and produce multiple copies of the plasmid in a cell. The plasmid may be pVAX, pCEP4 or pREP4 from Invitrogen (San Diego, CA), which may comprise the Epstein Barr virus origin of replication and nuclear antigen EBNA-1 coding region, which may produce high copy episomal replication without integration. The backbone of the plasmid may be pAV0242. The plasmid may be a replication defective adenovirus type 5 (Ad5) plasmid.

The plasmid may be pSE420 (Invitrogen, San Diego, Calif), which may be used for protein production in Escherichia coli (E. coli). The plasmid may also be p YES2 (Invitrogen, San Diego, Calif), which may be used for protein production in Saccharomyces cerevisiae strains of yeast. The plasmid may also be of the MAXBAC™ complete baculovirus expression system (Invitrogen, San Diego, Calif), which may be used for protein production in insect cells. The plasmid may also be pcDNAI or pcDNA3 (Invitrogen, San Diego, Calif), which may be used for protein production in mammalian cells such as Chinese hamster ovary (CHO) cells.

### (3) RNA Vectors

In one embodiment, the nucleic acid is an RNA molecule. In one embodiment, the RNA molecule is transcribed from a DNA sequence described herein. For example, in some embodiments, the RNA molecule is encoded by one of SEQ ID NOs:1, 3, 5, 7, 9, 11, 13, 15, 17, 19, 21, 23, 25, 27, or a variant thereof or a fragment thereof. In another embodiment, the nucleotide sequence comprises an RNA sequence transcribed by a DNA sequence encoding the polypeptide sequence of SEQ ID NOs:2, 4, 6, 8, 10, 12, 14, 16, 18, 20, 22, 24, 26, 28, or a variant thereof or a fragment thereof. Accordingly, in one embodiment, the invention provides an RNA molecule encoding one or more of the checkpoint inhibitors disclosed herein. The RNA may be plus-stranded. Accordingly, in some embodiments, the RNA molecule can be translated by cells without needing any intervening replication steps such as reverse transcription. A RNA molecule useful with the invention may have a 5′ cap (e.g. a 7-methylguanosine). This cap can enhance in vivo translation of the RNA. The 5′ nucleotide of a RNA molecule useful with the invention may have a 5′ triphosphate group. In a capped RNA this may be linked to a 7-methylguanosine via a 5′-to-5′ bridge. A RNA molecule may have a 3′ poly-A tail. It may also include a poly-A polymerase recognition sequence (e.g. AAUAAA) near its 3′ end. A RNA molecule useful with the invention may be single-stranded.

### (4) Circular and Linear Vector

The one or more vectors may be one or more circular plasmids, which may transform a target cell by integration into the cellular genome or exist extrachromosomally (e.g., autonomous replicating plasmid with an origin of replication). The vector can be pVAX, pcDNA3.0, or provax, or any other expression vector capable of expressing the heavy chain polypeptide and/or light chain polypeptide encoded by the recombinant nucleic acid sequence construct.

Also provided herein is a linear nucleic acid, or linear expression cassette (“LEC”), that is capable of being efficiently delivered to a subject via electroporation and expressing the heavy chain polypeptide and/or light chain polypeptide encoded by the recombinant nucleic acid sequence construct. The LEC may be any linear DNA devoid of any phosphate backbone. The DNA may encode one or more antibodies. The LEC may comprise a promoter, an intron, a stop codon, a polyadenylation signal. The LEC may not contain any antibiotic resistance genes and/or a phosphate backbone. The LEC may not contain other nucleic acid sequences unrelated to the desired gene expression. The LEC is capable of being efficiently delivered to a subject via electroporation and expressing one or more desired antibodies. The LEC may be derived from any plasmid capable of being linearized. These can also be made synthetically without bacterial growth and not from linearized sequences. The plasmid may be capable of expressing the heavy chain polypeptide and/or light chain polypeptide encoded by the recombinant nucleic acid sequence construct. The plasmid can be pNP (Puerto Rico/34) or pM2 (New Caledonia/99). The plasmid may be WLV009, pVAX, pcDNA3.0, or provax, or any other expression vector capable of expressing the heavy chain polypeptide and/or light chain polypeptide encoded by the recombinant nucleic acid sequence construct.

The LEC can be pcrM2. The LEC can be pcrNP. pcrNP and pcrMR can be derived from pNP (Puerto Rico/34) and pM2 (New Caledonia/99), respectively.

### (5) Viral Vectors

In one embodiment, viral vectors are provided herein which are capable of delivering a nucleic acid of the invention to a cell. The expression vector may be provided to a cell in the form of a viral vector. Viral vector technology is well known in the art and is described, for example, in Sambrook et al. (2001), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector comprises an origin of replication functional in at least one organism, a promoter sequence, convenient restriction endonuclease sites, and one or more selectable markers. (See, e.g., WO 01/96584; WO 01/29058; and U.S. Pat. No. 6,326,193. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

### (6) Method of Preparing the Vector

Provided herein is a method for preparing the one or more vectors in which the recombinant nucleic acid sequence construct has been placed. After the final subcloning step, the vector can be used to inoculate a cell culture in a large scale fermentation tank, using known methods in the art.

In other embodiments, after the final subcloning step, the vector can be used with one or more electroporation (EP) devices. The EP devices are described below in more detail.

The one or more vectors can be formulated or manufactured using a combination of known devices and techniques, but preferably they are manufactured using a plasmid manufacturing technique that is described in a licensed, co-pending U.S. provisional application U.S. Ser. No. 60/939,792, which was filed on May 23, 2007. In some examples, the DNA plasmids described herein can be formulated at concentrations greater than or equal to 10 mg/mL. The manufacturing techniques also include or incorporate various devices and protocols that are commonly known to those of ordinary skill in the art, in addition to those described in U.S. Ser. No. 60/939,792, including those described in a licensed patent, U.S. Pat. No. 7,238,522, which issued on Jul. 3, 2007. The above-referenced application and patent, U.S. Ser. No. 60/939,792 and U.S. Pat. No. 7,238,522, respectively, are hereby incorporated in their entirety.

### 3. ANTIBODY

As described above, the recombinant nucleic acid sequence can encode the antibody, a fragment thereof, a variant thereof, or a combination thereof. The antibody can bind or react with the antigen, which is described in more detail below.

The antibody can treat, prevent, and/or protect against disease in the subject administered a composition of the invention. The antibody by binding the antigen can treat, prevent, and/or protect against disease in the subject administered the composition. The antibody can promote survival of the disease in the subject administered the composition. In one embodiment, the antibody can provide increased survival of the disease in the subject over the expected survival of a subject having the disease who has not been administered the antibody. In various embodiments, the antibody can provide at least about a 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or a 100% increase in survival of the disease in subjects administered the composition over the expected survival in the absence of the composition. In one embodiment, the antibody can provide increased protection against the disease in the subject over the expected protection of a subject who has not been administered the antibody. In various embodiments, the antibody can protect against disease in at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% of subjects administered the composition over the expected protection in the absence of the composition.

The antibody may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region. Proceeding from the N-terminus of a heavy or light chain, these regions are denoted as “CDR1,” “CDR2,” and “CDR3,” respectively. An antigen-binding site, therefore, may include six CDRs, comprising the CDR set from each of a heavy and a light chain V region.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)2 fragment, which comprises both antigen-binding sites. Accordingly, the antibody can be the Fab or F(ab′)2. The Fab can include the heavy chain polypeptide and the light chain polypeptide. The heavy chain polypeptide of the Fab can include the VH region and the CH1 region. The light chain of the Fab can include the VL region and CL region.

The antibody can be an immunoglobulin (Ig). The Ig can be, for example, IgA, IgM, IgD, IgE, and IgG. The immunoglobulin can include the heavy chain polypeptide and the light chain polypeptide. The heavy chain polypeptide of the immunoglobulin can include a VH region, a CH1 region, a hinge region, a CH2 region, and a CH3 region. The light chain polypeptide of the immunoglobulin can include a VL region and CL region.

The antibody can be a polyclonal or monoclonal antibody. The antibody can be a chimeric antibody, a single chain antibody, an affinity matured antibody, a human antibody, a humanized antibody, or a fully human antibody. The humanized antibody can be an antibody from a non-human species that binds the desired antigen having one or more complementarity determining regions (CDRs) from the non-human species and framework regions from a human immunoglobulin molecule.

The antibody can be a bispecific antibody as described below in more detail. The antibody can be a bifunctional antibody as also described below in more detail.

As described above, the antibody can be generated in the subject upon administration of the composition to the subject. The antibody may have a half-life within the subject. In some embodiments, the antibody may be modified to extend or shorten its half-life within the subject. Such modifications are described below in more detail.

The antibody can be defucosylated as described in more detail below.

The antibody may be modified to reduce or prevent antibody-dependent enhancement (ADE) of disease associated with the antigen as described in more detail below.

a. Bispecific Antibody

The recombinant nucleic acid sequence can encode a bispecific antibody, a fragment thereof, a variant thereof, or a combination thereof. The bispecific antibody can bind or react with two antigens, for example, two of the antigens described below in more detail. The bispecific antibody can be comprised of fragments of two of the antibodies described herein, thereby allowing the bispecific antibody to bind or react with two desired target molecules, which may include the antigen, which is described below in more detail, a ligand, including a ligand for a receptor, a receptor, including a ligand-binding site on the receptor, a ligand-receptor complex, and a marker, including a cancer marker.

b. Bifunctional Antibody

The recombinant nucleic acid sequence can encode a bifunctional antibody, a fragment thereof, a variant thereof, or a combination thereof. The bifunctional antibody can bind or react with the antigen described below. The bifunctional antibody can also be modified to impart an additional functionality to the antibody beyond recognition of and binding to the antigen. Such a modification can include, but is not limited to, coupling to factor H or a fragment thereof. Factor H is a soluble regulator of complement activation and thus, may contribute to an immune response via complement-mediated lysis (CML).

c. Extension of Antibody Half-Life

As described above, the antibody may be modified to extend or shorten the half-life of the antibody in the subject. The modification may extend or shorten the half-life of the antibody in the serum of the subject.

The modification may be present in a constant region of the antibody. The modification may be one or more amino acid substitutions in a constant region of the antibody that extend the half-life of the antibody as compared to a half-life of an antibody not containing the one or more amino acid substitutions. The modification may be one or more amino acid substitutions in the CH2 domain of the antibody that extend the half-life of the antibody as compared to a half-life of an antibody not containing the one or more amino acid substitutions.

In some embodiments, the one or more amino acid substitutions in the constant region may include replacing a methionine residue in the constant region with a tyrosine residue, a serine residue in the constant region with a threonine residue, a threonine residue in the constant region with a glutamate residue, or any combination thereof, thereby extending the half-life of the antibody.

In other embodiments, the one or more amino acid substitutions in the constant region may include replacing a methionine residue in the CH2 domain with a tyrosine residue, a serine residue in the CH2 domain with a threonine residue, a threonine residue in the CH2 domain with a glutamate residue, or any combination thereof, thereby extending the half-life of the antibody.

d. Defucosylation

The recombinant nucleic acid sequence can encode an antibody that is not fucosylated (i.e., a defucosylated antibody or a non-fucosylated antibody), a fragment thereof, a variant thereof, or a combination thereof. Fucosylation includes the addition of the sugar fucose to a molecule, for example, the attachment of fucose to N-glycans, 0-glycans and glycolipids.

Accordingly, in a defucosylated antibody, fucose is not attached to the carbohydrate chains of the constant region. In turn, this lack of fucosylation may improve FcγRIIIa binding and antibody directed cellular cytotoxic (ADCC) activity by the antibody as compared to the fucosylated antibody. Therefore, in some embodiments, the non-fucosylated antibody may exhibit increased ADCC activity as compared to the fucosylated antibody.

The antibody may be modified so as to prevent or inhibit fucosylation of the antibody. In some embodiments, such a modified antibody may exhibit increased ADCC activity as compared to the unmodified antibody. The modification may be in the heavy chain, light chain, or a combination thereof. The modification may be one or more amino acid substitutions in the heavy chain, one or more amino acid substitutions in the light chain, or a combination thereof.

e. Reduced ADE Response

The antibody may be modified to reduce or prevent antibody-dependent enhancement (ADE) of disease associated with the antigen, but still neutralize the antigen.

In some embodiments, the antibody may be modified to include one or more amino acid substitutions that reduce or prevent binding of the antibody to FcγRla. The one or more amino acid substitutions may be in the constant region of the antibody. The one or more amino acid substitutions may include replacing a leucine residue with an alanine residue in the constant region of the antibody, i.e., also known herein as LA, LA mutation or LA substitution. The one or more amino acid substitutions may include replacing two leucine residues, each with an alanine residue, in the constant region of the antibody and also known herein as LALA, LALA mutation, or LALA substitution. The presence of the LALA substitutions may prevent or block the antibody from binding to FcγRla, and thus, the modified antibody does not enhance or cause ADE of disease associated with the antigen, but still neutralizes the antigen.

### 4. METHOD OF GENERATING THE SYNTHETIC ANTIBODY

The present invention also relates a method of generating the synthetic antibody. The method can include administering the composition to the subject in need thereof by using the method of delivery described in more detail below. Accordingly, the synthetic antibody is generated in the subject or in vivo upon administration of the composition to the subject.

The method can also include introducing the composition into one or more cells, and therefore, the synthetic antibody can be generated or produced in the one or more cells. The method can further include introducing the composition into one or more tissues, for example, but not limited to, skin and muscle, and therefore, the synthetic antibody can be generated or produced in the one or more tissues.

### 5. CANCER ANTIGEN

The compositions and methods of the invention can be used in combination with a vaccine comprising an antigen, or fragment or variant thereof.

Markers are known proteins that are present or upregulated vis-à-vis certain cancer cells. By methodology of generating antigens that represent such markers in a way to break tolerance to self, a cancer vaccine can be generated. Such cancer vaccines can include the checkpoint inhibitors to enhance the immune response. The following are some cancer antigens:

a. hTERT

hTERT is a human telomerase reverse transcriptase that synthesizes a TTAGGG tag on the end of telomeres to prevent cell death due to chromosomal shortening. Hyperproliferative cells with abnormally high expression of hTERT may be targeted by immunotherapy. Recent studies demonstrate that hTERT expression in dendritic cells transfected with hTERT genes can induce CD8+ cytotoxic T cells and elicit a CD4+ T cells in an antigen-specific fashion.

hTERT can be administered in vectors described herein, and combined with checkpoint inhibitors in various vaccination schedules, including that in the Example, below.

b. Prostate Antigens

The following are antigens capable of eliciting an immune response in a mammal against a prostate antigen. The consensus antigen can comprise epitopes that make them particularly effective as immunogens against prostate cancer cells can be induced. The consensus prostate antigen can comprise the full length translation product, a variant thereof, a fragment thereof or a combination thereof.

The prostate antigens can include one or more of the following: PSA antigen, PSMA antigen, STEAP antigen, PSCA antigen, Prostatic acid phosphatase (PAP) antigen, and other known prostate cancer markers. Proteins may comprise sequences homologous to the prostate antigens, fragments of the prostate antigens and proteins with sequences homologous to fragments of the prostate antigens.

The prostate antigens can be administered in vectors described herein, and combined with checkpoint inhibitors in various vaccination schedules, including that in the Example, below.

c. WT1

The antigen can be Wilm's tumor suppressor gene 1 (WT1), a fragment thereof, a variant thereof, or a combination thereof. WT1 is a transcription factor containing at the N-terminus, a proline/glutamine-rich DNA-binding domain and at the C-terminus, four zinc finger motifs. WT1 plays a role in the normal development of the urogenital system and interacts with numerous factors, for example, p53, a known tumor suppressor and the serine protease HtrA2, which cleaves WT1 at multiple sites after treatment with a cytotoxic drug.

Mutation of WT1 can lead to tumor or cancer formation, for example, Wilm's tumor or tumors expressing WT1. Wilm's tumor often forms in one or both kidneys before metastasizing to other tissues, for example, but not limited to, liver tissue, urinary tract system tissue, lymph tissue, and lung tissue. Accordingly, Wilm's tumor can be considered a metastatic tumor. Wilm's tumor usually occurs in younger children (e.g., less than 5 years old) and in both sporadic and hereditary forms. Accordingly, the vaccine can be used for treating subjects suffering from Wilm's tumor. The vaccine can also be used for treating subjects with cancers or tumors that express WT1 for preventing development of such tumors in subjects. The WT1 antigen can differ from the native, “normal” WT1 gene, and thus, provide therapy or prophylaxis against an WT1 antigen-expressing tumor. Proteins may comprise sequences homologous to the WT1 antigens, fragments of the WT1 antigens and proteins with sequences homologous to fragments of the WT1 antigens.

The WT1 antigens can be administered in vectors described herein, and combined with checkpoint inhibitors in various vaccination schedules, including that in the Example, below.

d. Tyrosinase Antigen

The antigen tyrosinase (Tyr) antigen is an important target for immune mediated clearance by inducing (1) humoral immunity via B cell responses to generate antibodies that block monocyte chemoattractant protein-1 (MCP-1) production, thereby retarding myeloid derived suppressor cells (MDSCs) and suppressing tumor growth; (2) increase cytotoxic T lymphocyte such as CD8+(CTL) to attack and kill tumor cells; (3) increase T helper cell responses; (4) and increase inflammatory responses via IFN-γ and TFN-α or preferably all of the aforementioned.

Tyrosinase is a copper-containing enzyme that can be found in plant and animal tissues. Tyrosinase catalyzes the production of melanin and other pigments by the oxidation of phenols such as tyrosine. In melanoma, tyrosinase can become unregulated, resulting in increased melanin synthesis. Tyrosinase is also a target of cytotoxic T cell recognition in subjects suffering from melanoma. Accordingly, tyrosinase can be an antigen associated with melanoma.

The antigen can comprise protein epitopes that make them particularly effective as immunogens against which anti-Tyr immune responses can be induced. The Tyr antigen can comprise the full length translation product, a variant thereof, a fragment thereof or a combination thereof.

The Tyr antigen can comprise a consensus protein. The Tyr antigen induces antigen-specific T-cell and high titer antibody responses both systemically against all cancer and tumor related cells. As such, a protective immune response is provided against tumor formation by vaccines comprising the Tyr consensus antigen. Accordingly, any user can design a vaccine of the present invention to include a Tyr antigen to provide broad immunity against tumor formation, metastasis of tumors, and tumor growth. Proteins may comprise sequences homologous to the Tyr antigens, fragments of the Tyr antigens and proteins with sequences homologous to fragments of the Tyr antigens.

The Tyr antigens can be administered in vectors described herein, and combined with checkpoint inhibitors in various vaccination schedules, including that in the Example, below.

e. NYESO1

NY-ESO-1 is a cancer-testis antigen expressed in various cancers where it can induce both cellular and humoral immunity. Gene expression studies have shown upregulation of the gene for NY-ESO-1, CTAGlB, in myxoid and round cell liposarcomas. Proteins may comprise sequences homologous to the NYESO1 antigens, fragments of the NYESO1 antigens and proteins with sequences homologous to fragments of the NYESO1 antigens.

The NYESO1 antigens can be administered in vectors described herein, and combined with checkpoint inhibitors in various vaccination schedules, including that in the Example, below.

f. PRAME

Melanoma antigen preferentially expressed in tumors (PRAME antigen) is a protein that in humans is encoded by the PRAME gene. This gene encodes an antigen that is predominantly expressed in human melanomas and that is recognized by cytolytic T lymphocytes. It is not expressed in normal tissues, except testis. The gene is also expressed in acute leukemias. Five alternatively spliced transcript variants encoding the same protein have been observed for this gene. Proteins may comprise sequences homologous to the PRAME antigens, fragments of the PRAME antigens and proteins with sequences homologous to fragments of the PRAME antigens.

The PRAME antigens can be administered in vectors described herein, and combined with checkpoint inhibitors in various vaccination schedules, including that in the Example, below.

g. MAGE

MAGE stands for Melanoma-associated Antigen, and in particular melanoma associated antigen 4 (MAGEA4). MAGE-A4 is expressed in male germ cells and tumor cells of various histological types such as gastrointestinal, esophageal and pulmonary carcinomas. MAGE-A4 binds the oncoprotein, Gankyrin. This MAGE-A4 specific binding is mediated by its C-terminus. Studies have shown that exogenous MAGE-A4 can partly inhibit the adhesion-independent growth of Gankyrin-overexpressing cells in vitro and suppress the formation of migrated tumors from these cells in nude mice. This inhibition is dependent upon binding between MAGE-A4 and Gankyrin, suggesting that interactions between Gankyrin and MAGE-A4 inhibit Gankyrin-mediated carcinogenesis. It is likely that MAGE expression in tumor tissue is not a cause, but a result of tumor genesis, and MAGE genes take part in the immune process by targeting early tumor cells for destruction.

Melanoma-associated antigen 4 protein (MAGEA4) can be involved in embryonic development and tumor transformation and/or progression. MAGEA4 is normally expressed in testes and placenta. MAGEA4, however, can be expressed in many different types of tumors, for example, melanoma, head and neck squamous cell carcinoma, lung carcinoma, and breast carcinoma. Accordingly, MAGEA4 can be antigen associated with a variety of tumors.

The MAGEA4 antigen can induce antigen-specific T cell and/or high titer antibody responses, thereby inducing or eliciting an immune response that is directed to or reactive against the cancer or tumor expressing the antigen. In some embodiments, the induced or elicited immune response can be a cellular, humoral, or both cellular and humoral immune responses. In some embodiments, the induced or elicited cellular immune response can include induction or secretion of interferon-gamma (IFN-γ) and/or tumor necrosis factor alpha (TNF-α). In other embodiments, the induced or elicited immune response can reduce or inhibit one or more immune suppression factors that promote growth of the tumor or cancer expressing the antigen, for example, but not limited to, factors that down regulate MHC presentation, factors that up regulate antigen-specific regulatory T cells (Tregs), PD-L1, FasL, cytokines such as IL-10 and TFG-β, tumor associated macrophages, tumor associated fibroblasts.

The MAGEA4 antigen can comprise protein epitopes that make them particularly effective as immunogens against which anti-MAGEA4 immune responses can be induced. The MAGEA4 antigen can comprise the full length translation product, a variant thereof, a fragment thereof or a combination thereof. The MAGEA4 antigen can comprise a consensus protein.

The nucleic acid sequence encoding the consensus MAGEA4 antigen can be optimized with regards to codon usage and corresponding RNA transcripts. The nucleic acid encoding the consensus MAGEA4 antigen can be codon and RNA optimized for expression. In some embodiments, the nucleic acid sequence encoding the consensus MAGEA4 antigen can include a Kozak sequence (e.g., GCC ACC) to increase the efficiency of translation. The nucleic acid encoding the consensus MAGEA4 antigen can include multiple stop codons (e.g., TGA TGA) to increase the efficiency of translation termination.

The MAGE antigens can be administered in vectors described herein, and combined with checkpoint inhibitors in various vaccination schedules, including that in the Example, below.

h. Tumor Antigen

In the context of the present invention, “tumor antigen” or “hyperproliferative disorder antigen” or “antigen associated with a hyperproliferative disorder,” refers to antigens that are common to specific hyperproliferative disorders such as cancer. The antigens discussed herein are merely included by way of example. The list is not intended to be exclusive and further examples will be readily apparent to those of skill in the art.

Tumor antigens are proteins that are produced by tumor cells that elicit an immune response, particularly T-cell mediated immune responses. The selection of the antigen binding moiety of the invention will depend on the particular type of cancer to be treated. Tumor antigens are well known in the art and include, for example, a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

In one embodiment, the tumor antigen comprises one or more antigenic cancer epitopes associated with a malignant tumor. Malignant tumors express a number of proteins that can serve as target antigens for an immune attack. These molecules include but are not limited to tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecules such as the oncogene HER-2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD19, CD20 and CD37 are other candidates for target antigens in B-cell lymphoma. Some of these antigens (CEA, HER-2, CD19, CD20, idiotype) have been used as targets for passive immunotherapy with monoclonal antibodies with limited success.

The type of tumor antigen referred to in the invention may also be a tumor-specific antigen (TSA) or a tumor-associated antigen (TAA). A TSA is unique to tumor cells and does not occur on other cells in the body. A TAA associated antigen is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

Non-limiting examples of TSA or TAA antigens include the following: Differentiation antigens such as MART-1/MelanA (MART-I), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3CA 27.29BCAA, CA 195, CA 242, CA-50, CAM43, CD68P1, CO-029, FGF-5, G250, Ga733EpCAM, HTgp-175, M344, MA-50, MG7-Ag, MOV18, NB/70K, NY-CO-1, RCAS1, SDCCAGi6, TA-90Mac-2 binding proteincyclophilin C-associated protein, TAAL6, TAG72, TLP, and TPS.

a. Excipients and Other Components of the Vaccine

The vaccine may further comprise a pharmaceutically acceptable excipient. The pharmaceutically acceptable excipient can be functional molecules such as vehicles, adjuvants, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and the poly-L-glutamate may be present in the vaccine at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the genetic construct. The DNA plasmid vaccines may also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. The transfection facilitating agent is a polyanion, polycation, including poly-L-glutamate (LGS), or lipid. Concentration of the transfection agent in the vaccine is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The pharmaceutically acceptable excipient can be an adjuvant in addition to the checkpoint inhibitor antibodies of the invention. The additional adjuvant can be other genes that are expressed in an alternative plasmid or are delivered as proteins in combination with the plasmid above in the vaccine. The adjuvant may be selected from the group consisting of: α-interferon(IFN-α), β-interferon (IFN-β), γ-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80, CD86 including IL-15 having the signal sequence deleted and optionally including the signal peptide from IgE. The adjuvant can be IL-12, IL-15, IL-28, CTACK, TECK, platelet derived growth factor (PDGF), TNFa, TNFβ, GM-CSF, epidermal growth factor (EGF), IL-1, IL-2, IL-4, IL-5, PD-1, IL-10, IL-12, IL-18, or a combination thereof.

Other genes that can be useful as adjuvants in addition to the antibodies of the invention include those encoding: MCP-1, MIP-1a, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, IL-22, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

The vaccine may further comprise a genetic vaccine facilitator agent as described in U.S. Serial No. 021,579 filed Apr. 1, 1994, which is fully incorporated by reference.

The vaccine can be formulated according to the mode of administration to be used. An injectable vaccine pharmaceutical composition can be sterile, pyrogen free and particulate free. An isotonic formulation or solution can be used. Additives for isotonicity can include sodium chloride, dextrose, mannitol, sorbitol, and lactose. The vaccine can comprise a vasoconstriction agent. The isotonic solutions can include phosphate buffered saline. Vaccine can further comprise stabilizers including gelatin and albumin. The stabilizers can allow the formulation to be stable at room or ambient temperature for extended periods of time, including LGS or polycations or polyanions.

### 6. METHOD OF VACCINATION

The present invention is also directed to a method of increasing an immune response in a subject. Increasing the immune response can be used to treat and/or prevent disease in the subject. The method can include administering the herein disclosed vaccine to the subject. The subject administered the vaccine can have an increased or boosted immune response as compared to a subject administered the antigen alone. In some embodiments, the immune response can be increased by about 0.5-fold to about 15-fold, about 0.5-fold to about 10-fold, or about 0.5-fold to about 8-fold. Alternatively, the immune response in the subject administered the vaccine can be increased by at least about 0.5-fold, at least about 1.0-fold, at least about 1.5-fold, at least about 2.0-fold, at least about 2.5-fold, at least about 3.0-fold, at least about 3.5-fold, at least about 4.0-fold, at least about 4.5-fold, at least about 5.0-fold, at least about 5.5-fold, at least about 6.0-fold, at least about 6.5-fold, at least about 7.0-fold, at least about 7.5-fold, at least about 8.0-fold, at least about 8.5-fold, at least about 9.0-fold, at least about 9.5-fold, at least about 10.0-fold, at least about 10.5-fold, at least about 11.0-fold, at least about 11.5-fold, at least about 12.0-fold, at least about 12.5-fold, at least about 13.0-fold, at least about 13.5-fold, at least about 14.0-fold, at least about 14.5-fold, or at least about 15.0-fold.

In still other alternative embodiments, the immune response in the subject administered the vaccine can be increased about 50% to about 1500%, about 50% to about 1000%, or about 50% to about 800%. In other embodiments, the immune response in the subject administered the vaccine can be increased by at least about 50%, at least about 100%, at least about 150%, at least about 200%, at least about 250%, at least about 300%, at least about 350%, at least about 400%, at least about 450%, at least about 500%, at least about 550%, at least about 600%, at least about 650%, at least about 700%, at least about 750%, at least about 800%, at least about 850%, at least about 900%, at least about 950%, at least about 1000%, at least about 1050%, at least about 1100%, at least about 1150%, at least about 1200%, at least about 1250%, at least about 1300%, at least about 1350%, at least about 1450%, or at least about 1500%.

The vaccine dose can be between 1 μg to 10 mg active component/kg body weight/time, and can be 20 μg to 10 mg component/kg body weight/time. The vaccine can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of vaccine doses for effective treatment can be 1, 2, 3, 4, 5, 6, 7, 8, 9, or 10.

a. Administration

The composition of the invention can be formulated in accordance with standard techniques well known to those skilled in the pharmaceutical art. Such compositions can be administered in dosages and by techniques well known to those skilled in the medical arts taking into consideration such factors as the age, sex, weight, and condition of the particular subject, and the route of administration. The subject can be a mammal, such as a human, a horse, a cow, a pig, a sheep, a cat, a dog, a rat, or a mouse.

The composition of the invention can be administered prophylactically or therapeutically. In prophylactic administration, the vaccines can be administered in an amount sufficient to induce an immune response. In therapeutic applications, the compositions of the invention are administered to a subject in need thereof in an amount sufficient to elicit a therapeutic effect. An amount adequate to accomplish this is defined as “therapeutically effective dose.” Amounts effective for this use will depend on, e.g., the particular composition of the vaccine regimen administered, the manner of administration, the stage and severity of the disease, the general state of health of the patient, and the judgment of the prescribing physician.

The composition of the invention can be administered by methods well known in the art as described in Donnelly et al. (Ann. Rev. Immunol. 15:617-648 (1997)); Felgner et al. (U.S. Pat. No. 5,580,859, issued Dec. 3, 1996); Felgner (U.S. Pat. No. 5,703,055, issued Dec. 30, 1997); and Carson et al. (U.S. Pat. No. 5,679,647, issued Oct. 21, 1997), the contents of all of which are incorporated herein by reference in their entirety. The DNA of the composition of the invention can be complexed to particles or beads that can be administered to an individual, for example, using a vaccine gun. One skilled in the art would know that the choice of a pharmaceutically acceptable carrier, including a physiologically acceptable compound, depends, for example, on the route of administration of the expression vector.

The composition of the invention can be delivered via a variety of routes. Typical delivery routes include parenteral administration, e.g., intradermal, intramuscular or subcutaneous delivery. Other routes include oral administration, intranasal, and intravaginal routes. For the DNA of the composition of the invention in particular, the composition can be delivered to the interstitial spaces of tissues of an individual (Felgner et al., U.S. Pat. Nos. 5,580,859 and 5,703,055, the contents of all of which are incorporated herein by reference in their entirety). The composition can also be administered to muscle, or can be administered via intradermal or subcutaneous injections, or transdermally, such as by iontophoresis. Epidermal administration of the composition can also be employed. Epidermal administration can involve mechanically or chemically irritating the outermost layer of epidermis to stimulate an immune response to the irritant (Carson et al., U.S. Pat. No. 5,679,647, the contents of which are incorporated herein by reference in its entirety).

The composition of the invention can also be formulated for administration via the nasal passages. Formulations suitable for nasal administration, wherein the carrier is a solid, can include a coarse powder having a particle size, for example, in the range of about 10 to about 500 microns which is administered in the manner in which snuff is taken, i.e., by rapid inhalation through the nasal passage from a container of the powder held close up to the nose. The formulation can be a nasal spray, nasal drops, or by aerosol administration by nebulizer. The formulation can include aqueous or oily solutions of the vaccine.

The composition of the invention can be a liquid preparation such as a suspension, syrup or elixir. The composition of the invention can also be a preparation for parenteral, subcutaneous, intradermal, intramuscular or intravenous administration (e.g., injectable administration), such as a sterile suspension or emulsion.

The composition of the invention can be incorporated into liposomes, microspheres or other polymer matrices (Felgner et al., U.S. Pat. No. 5,703,055; Gregoriadis, Liposome Technology, Vols. Ito III (2nd ed. 1993), the contents of which are incorporated herein by reference in their entirety). Liposomes can consist of phospholipids or other lipids, and can be nontoxic, physiologically acceptable and metabolizable carriers that are relatively simple to make and administer.

The composition of the invention can be administered via electroporation, such as by a method described in U.S. Pat. No. 7,664,545, the contents of which are incorporated herein by reference. The electroporation can be by a method and/or apparatus described in U.S. Pat. Nos. 6,302,874; 5,676,646; 6,241,701; 6,233,482; 6,216,034; 6,208,893; 6,192,270; 6,181,964; 6,150,148; 6,120,493; 6,096,020; 6,068,650; and 5,702,359, the contents of which are incorporated herein by reference in their entirety. The electroporation may be carried out via a minimally invasive device.

The minimally invasive electroporation device (“MID”) may be an apparatus for injecting the vaccine described above and associated fluid into body tissue. The device may comprise a hollow needle, DNA cassette, and fluid delivery means, wherein the device is adapted to actuate the fluid delivery means in use so as to concurrently (for example, automatically) inject DNA into body tissue during insertion of the needle into the said body tissue. This has the advantage that the ability to inject the DNA and associated fluid gradually while the needle is being inserted leads to a more even distribution of the fluid through the body tissue. The pain experienced during injection may be reduced due to the distribution of the DNA being injected over a larger area.

The MID may inject the vaccine into tissue without the use of a needle. The MID may inject the vaccine as a small stream or jet with such force that the vaccine pierces the surface of the tissue and enters the underlying tissue and/or muscle. The force behind the small stream or jet may be provided by expansion of a compressed gas, such as carbon dioxide through a micro-orifice within a fraction of a second. Examples of minimally invasive electroporation devices, and methods of using them, are described in published U.S. Patent Application No. 20080234655; U.S. Pat. Nos. 6,520,950; 7,171,264; 6,208,893; 6,009,347; 6,120,493; 7,245,963; 7,328,064; and 6,763,264, the contents of each of which are herein incorporated by reference.

The MID may comprise an injector that creates a high-speed jet of liquid that painlessly pierces the tissue. Such needle-free injectors are commercially available. Examples of needle-free injectors that can be utilized herein include those described in U.S. Pat. Nos. 3,805,783; 4,447,223; 5,505,697; and 4,342,310, the contents of each of which are herein incorporated by reference.

A desired composition of the invention in a form suitable for direct or indirect electrotransport may be introduced (e.g., injected) using a needle-free injector into the tissue to be treated, usually by contacting the tissue surface with the injector so as to actuate delivery of a jet of the agent, with sufficient force to cause penetration of the vaccine into the tissue. For example, if the tissue to be treated is mucosa, skin or muscle, the agent is projected towards the mucosal or skin surface with sufficient force to cause the agent to penetrate through the stratum corneum and into dermal layers, or into underlying tissue and muscle, respectively.

Needle-free injectors are well suited to deliver vaccines to all types of tissues, particularly to skin and mucosa. In some embodiments, a needle-free injector may be used to propel a liquid that contains the vaccine to the surface and into the subject's skin or mucosa. Representative examples of the various types of tissues that can be treated using the invention methods include pancreas, larynx, nasopharynx, hypopharynx, oropharynx, lip, throat, lung, heart, kidney, muscle, breast, colon, prostate, thymus, testis, skin, mucosal tissue, ovary, blood vessels, or any combination thereof.

The MID may have needle electrodes that electroporate the tissue. By pulsing between multiple pairs of electrodes in a multiple electrode array, for example set up in rectangular or square patterns, provides improved results over that of pulsing between a pair of electrodes. Disclosed, for example, in U.S. Pat. No. 5,702,359 entitled “Needle Electrodes for Mediated Delivery of Drugs and Genes” is an array of needles wherein a plurality of pairs of needles may be pulsed during the therapeutic treatment. In that application, which is incorporated herein by reference as though fully set forth, needles were disposed in a circular array, but have connectors and switching apparatus enabling a pulsing between opposing pairs of needle electrodes. A pair of needle electrodes for delivering recombinant expression vectors to cells may be used. Such a device and system is described in U.S. Pat. No. 6,763,264, the contents of which are herein incorporated by reference. Alternatively, a single needle device may be used that allows injection of the DNA and electroporation with a single needle resembling a normal injection needle and applies pulses of lower voltage than those delivered by presently used devices, thus reducing the electrical sensation experienced by the patient.

The MID may comprise one or more electrode arrays. The arrays may comprise two or more needles of the same diameter or different diameters. The needles may be evenly or unevenly spaced apart. The needles may be between 0.005 inches and 0.03 inches, between 0.01 inches and 0.025 inches; or between 0.015 inches and 0.020 inches. The needle may be 0.0175 inches in diameter. The needles may be 0.5 mm, 1.0 mm, 1.5 mm, 2.0 mm, 2.5 mm, 3.0 mm, 3.5 mm, 4.0 mm, or more spaced apart.

The MID may consist of a pulse generator and a two or more-needle vaccine injectors that deliver the vaccine and electroporation pulses in a single step. The pulse generator may allow for flexible programming of pulse and injection parameters via a flash card operated personal computer, as well as comprehensive recording and storage of electroporation and patient data. The pulse generator may deliver a variety of volt pulses during short periods of time. For example, the pulse generator may deliver three 15 volt pulses of 100 ms in duration. An example of such a MID is the Elgen 1000 system by Inovio Biomedical Corporation, which is described in U.S. Pat. No. 7,328,064, the contents of which are herein incorporated by reference.

The MID may be a CELLECTRA (Inovio Pharmaceuticals, Plymouth Meeting, PA) device and system, which is a modular electrode system, that facilitates the introduction of a macromolecule, such as a DNA, into cells of a selected tissue in a body or plant. The modular electrode system may comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The macromolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the macromolecule into the cell between the plurality of electrodes. Cell death due to overheating of cells is minimized by limiting the power dissipation in the tissue by virtue of constant-current pulses. The Cellectra device and system is described in U.S. Pat. No. 7,245,963, the contents of which are herein incorporated by reference.

The MID may be an Elgen 1000 system (Inovio Pharmaceuticals). The Elgen 1000 system may comprise device that provides a hollow needle; and fluid delivery means, wherein the apparatus is adapted to actuate the fluid delivery means in use so as to concurrently (for example automatically) inject fluid, the described vaccine herein, into body tissue during insertion of the needle into the said body tissue. The advantage is the ability to inject the fluid gradually while the needle is being inserted leads to a more even distribution of the fluid through the body tissue. It is also believed that the pain experienced during injection is reduced due to the distribution of the volume of fluid being injected over a larger area.

In addition, the automatic injection of fluid facilitates automatic monitoring and registration of an actual dose of fluid injected. This data can be stored by a control unit for documentation purposes if desired.

It will be appreciated that the rate of injection could be either linear or non-linear and that the injection may be carried out after the needles have been inserted through the skin of the subject to be treated and while they are inserted further into the body tissue.

Suitable tissues into which fluid may be injected by the apparatus of the present invention include tumor tissue, skin or liver tissue but may be muscle tissue.

The apparatus further comprises needle insertion means for guiding insertion of the needle into the body tissue. The rate of fluid injection is controlled by the rate of needle insertion. This has the advantage that both the needle insertion and injection of fluid can be controlled such that the rate of insertion can be matched to the rate of injection as desired. It also makes the apparatus easier for a user to operate. If desired means for automatically inserting the needle into body tissue could be provided.

A user could choose when to commence injection of fluid. Ideally however, injection is commenced when the tip of the needle has reached muscle tissue and the apparatus may include means for sensing when the needle has been inserted to a sufficient depth for injection of the fluid to commence. This means that injection of fluid can be prompted to commence automatically when the needle has reached a desired depth (which will normally be the depth at which muscle tissue begins). The depth at which muscle tissue begins could for example be taken to be a preset needle insertion depth such as a value of 4 mm which would be deemed sufficient for the needle to get through the skin layer.

The sensing means may comprise an ultrasound probe. The sensing means may comprise a means for sensing a change in impedance or resistance. In this case, the means may not as such record the depth of the needle in the body tissue but will rather be adapted to sense a change in impedance or resistance as the needle moves from a different type of body tissue into muscle. Either of these alternatives provides a relatively accurate and simple to operate means of sensing that injection may commence. The depth of insertion of the needle can further be recorded if desired and could be used to control injection of fluid such that the volume of fluid to be injected is determined as the depth of needle insertion is being recorded.

The apparatus may further comprise: a base for supporting the needle; and a housing for receiving the base therein, wherein the base is moveable relative to the housing such that the needle is retracted within the housing when the base is in a first rearward position relative to the housing and the needle extends out of the housing when the base is in a second forward position within the housing. This is advantageous for a user as the housing can be lined up on the skin of a patient, and the needles can then be inserted into the patient's skin by moving the housing relative to the base.

As stated above, it is desirable to achieve a controlled rate of fluid injection such that the fluid is evenly distributed over the length of the needle as it is inserted into the skin. The fluid delivery means may comprise piston driving means adapted to inject fluid at a controlled rate. The piston driving means could for example be activated by a servo motor.

However, the piston driving means may be actuated by the base being moved in the axial direction relative to the housing. It will be appreciated that alternative means for fluid delivery could be provided. Thus, for example, a closed container which can be squeezed for fluid delivery at a controlled or non-controlled rate could be provided in the place of a syringe and piston system.

The apparatus described above could be used for any type of injection. It is however envisaged to be particularly useful in the field of electroporation and so it may further comprises means for applying a voltage to the needle. This allows the needle to be used not only for injection but also as an electrode during, electroporation. This is particularly advantageous as it means that the electric field is applied to the same area as the injected fluid. There has traditionally been a problem with electroporation in that it is very difficult to accurately align an electrode with previously injected fluid and so user's have tended to inject a larger volume of fluid than is required over a larger area and to apply an electric field over a higher area to attempt to guarantee an overlap between the injected substance and the electric field. Using the present invention, both the volume of fluid injected and the size of electric field applied may be reduced while achieving a good fit between the electric field and the fluid.

### 7. CANCER THERAPY

The invention provides methods of treating or preventing cancer, or of treating and preventing metastasis of tumors. Related aspects of the invention provide methods of preventing, aiding in the prevention, and/or reducing metastasis of hyperplastic or tumor cells in an individual.

One aspect of the invention provides a method of inhibiting metastasis in an individual in need thereof, the method comprising administering to the individual an effective amount of a composition of the invention. The invention further provides a method of inhibiting metastasis in an individual in need thereof, the method comprising administering to the individual an effective metastasis-inhibiting amount of any one of the compositions described herein.

In some embodiments of treating or preventing cancer, or of treating and preventing metastasis of tumors in an individual in need thereof, a second agent is administered to the individual, such as an antineoplastic agent. In some embodiments, the second agent comprises a second metastasis-inhibiting agent, such as a plasminogen antagonist, or an adenosine deaminase antagonist. In other embodiments, the second agent is an angiogenesis inhibiting agent.

The compositions of the invention can be used to prevent, abate, minimize, control, and/or lessen cancer in humans and animals. The compositions of the invention can also be used to slow the rate of primary tumor growth. The compositions of the invention when administered to a subject in need of treatment can be used to stop the spread of cancer cells.

As such, the compositions of the invention can be administered as part of a combination therapy with one or more drugs or other pharmaceutical agents. When used as part of the combination therapy, the decrease in metastasis and reduction in primary tumor growth afforded by the compositions of the invention allows for a more effective and efficient use of any pharmaceutical or drug therapy being used to treat the patient. In addition, control of metastasis by the compositions of the invention affords the subject a greater ability to concentrate the disease in one location.

In one embodiment, the invention provides methods for preventing metastasis of malignant tumors or other cancerous cells as well as to reduce the rate of tumor growth. The methods comprise administering an effective amount of one or more of the compositions of the invention to a subject diagnosed with a malignant tumor or cancerous cells or to a subject having a tumor or cancerous cells.

The following are non-limiting examples of cancers that can be treated by the methods and compositions of the invention: Acute Lymphoblastic; Acute Myeloid Leukemia; Adrenocortical Carcinoma; Adrenocortical Carcinoma, Childhood; Appendix Cancer; Basal Cell Carcinoma; Bile Duct Cancer, Extrahepatic; Bladder Cancer; Bone Cancer; Osteosarcoma and Malignant Fibrous Histiocytoma; Brain Stem Glioma, Childhood; Brain Tumor, Adult; Brain Tumor, Brain Stem Glioma, Childhood; Brain Tumor, Central Nervous System Atypical Teratoid/Rhabdoid Tumor, Childhood; Central Nervous System Embryonal Tumors; Cerebellar Astrocytoma; Cerebral Astrocytotna/Malignant Glioma; Craniopharyngioma; Ependymoblastoma; Ependymoma; Medulloblastoma; Medulloepithelioma; Pineal Parenchymal Tumors of intermediate Differentiation; Supratentorial Primitive Neuroectodermal Tumors and Pineoblastoma; Visual Pathway and Hypothalamic Glioma; Brain and Spinal Cord Tumors; Breast Cancer; Bronchial Tumors; Burkitt Lymphoma; Carcinoid Tumor; Carcinoid Tumor, Gastrointestinal; Central Nervous System Atypical Teratoid/Rhabdoid Tumor; Central Nervous System Embryonal Tumors; Central Nervous System Lymphoma; Cerebellar Astrocytoma Cerebral Astrocytoma/Malignant Glioma, Childhood; Cervical Cancer; Chordoma, Childhood; Chronic Lymphocytic Leukemia; Chronic Myelogenous Leukemia; Chronic Myeloproliferative Disorders; Colon Cancer; Colorectal Cancer; Craniopharyngioma; Cutaneous T-Cell Lymphoma; Esophageal Cancer; Ewing Family of Tumors; Extragonadal Germ Cell Tumor; Extrahepatic Bile Duct Cancer; Eye Cancer, intraocular Melanoma; Eye Cancer, Retinoblastoma; Gallbladder Cancer; Gastric (Stomach) Cancer; Gastrointestinal Carcinoid Tumor; Gastrointestinal Stromal Tumor (GIST); Germ Cell Tumor, Extracranial; Germ Cell Tumor, Extragonadal; Germ Cell Tumor, Ovarian; Gestational Trophoblastic Tumor; Glioma; Glioma, Childhood Brain Stem; Glioma, Childhood Cerebral Astrocytoma; Glioma, Childhood Visual Pathway and Hypothalamic; Hairy Cell Leukemia; Head and Neck Cancer; Hepatocellular (Liver) Cancer; Histiocytosis, Langerhans Cell; Hodgkin Lymphoma; Hypopharyngeal Cancer; Hypothalamic and Visual Pathway Glioma; intraocular Melanoma; Islet Cell Tumors; Kidney (Renal Cell) Cancer; Langerhans Cell Histiocytosis; Laryngeal Cancer; Leukemia, Acute Lymphoblastic; Leukemia, Acute Myeloid; Leukemia, Chronic Lymphocytic; Leukemia, Chronic Myelogenous; Leukemia, Hairy Cell; Lip and Oral Cavity Cancer; Liver Cancer; Lung Cancer, Non-Small Cell; Lung Cancer, Small Cell; Lymphoma, AIDS-Related; Lymphoma, Burkitt; Lymphoma, Cutaneous T-Cell; Lymphoma, Hodgkin; Lymphoma, Non-Hodgkin; Lymphoma, Primary Central Nervous System; Macroglobulinemia, Waldenstrom; Malignant Fibrous Histiocvtoma of Bone and Osteosarcoma; Medulloblastoma; Melanoma; Melanoma, intraocular (Eye); Merkel Cell Carcinoma; Mesothelioma; Metastatic Squamous Neck Cancer with Occult Primary; Mouth Cancer; Multiple Endocrine Neoplasia Syndrome, (Childhood); Multiple Myeloma/Plasma Cell Neoplasm; Mycosis; Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple; Myeloproliferative Disorders, Chronic; Nasal Cavity and Paranasal Sinus Cancer; Nasopharyngeal Cancer; Neuroblastoma; Non-Small Cell Lung Cancer; Oral Cancer; Oral Cavity Cancer; Oropharyngeal Cancer; Osteosarcoma and Malignant Fibrous Histiocytoma of Bone; Ovarian Cancer; Ovarian Epithelial Cancer; Ovarian Germ Cell Tumor; Ovarian Low Malignant Potential Tumor; Pancreatic Cancer; Pancreatic Cancer, Islet Cell Tumors; Papillomatosis; Parathyroid Cancer; Penile Cancer; Pharyngeal Cancer; Pheochromocytoma; Pineal Parenchymal Tumors of Intermediate Differentiation; Pineoblastoma and Supratentorial Primitive Neuroectodermal Tumors; Pituitary Tumor; Plasma Celt Neoplasm/Multiple Myeloma; Pleuropulmonary Blastoma; Primary Central Nervous System Lymphoma; Prostate Cancer; Rectal Cancer; Renal Cell (Kidney) Cancer; Renal Pelvis and Ureter, Transitional Cell Cancer; Respiratory Tract Carcinoma Involving the NUT Gene on Chromosome 15; Retinoblastoma; Rhabdomyosarcoma; Salivary Gland Cancer; Sarcoma, Ewing Family of Tumors; Sarcoma, Kaposi; Sarcoma, Soft Tissue; Sarcoma, Uterine; Sezary Syndrome; Skin Cancer (Nonmelanoma); Skin Cancer (Melanoma); Skin Carcinoma, Merkel Cell; Small Cell Lung Cancer; Small Intestine Cancer; Soft Tissue Sarcoma; Squamous Cell Carcinoma, Squamous Neck Cancer with Occult Primary, Metastatic; Stomach (Gastric) Cancer; Supratentorial Primitive Neuroectodermal Tumors; T-Cell Lymphoma, Cutaneous; Testicular Cancer; Throat Cancer; Thymoma and Thymic Carcinoma; Thyroid Cancer; Transitional Cell Cancer of the Renal Pelvis and Ureter; Trophoblastic Tumor, Gestational; Urethral Cancer; Uterine Cancer, Endometrial; Uterine Sarcoma; Vaginal Cancer; Vulvar Cancer; Waldenstrom Macroglobulinemia; and Wilms Tumor.

In one embodiment, the invention provides a method to treat cancer metastasis comprising treating the subject prior to, concurrently with, or subsequently to the treatment with a composition of the invention, with a complementary therapy for the cancer, such as surgery, chemotherapy, chemotherapeutic agent, radiation therapy, or hormonal therapy or a combination thereof.

Chemotherapeutic agents include cytotoxic agents (e.g., 5-fluorouracil, cisplatin, carboplatin, methotrexate, daunorubicin, doxorubicin, vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic alkylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium).

Antiproliferative agents are compounds that decrease the proliferation of cells. Antiproliferative agents include alkylating agents, antimetabolites, enzymes, biological response modifiers, miscellaneous agents, hormones and antagonists, androgen inhibitors (e.g., flutamide and leuprolide acetate), antiestrogens (e.g., tamoxifen citrate and analogs thereof, toremifene, droloxifene and roloxifene), Additional examples of specific antiproliferative agents include, but are not limited to levamisole, gallium nitrate, granisetron, sargramostim strontium-89 chloride, filgrastim, pilocarpine, dexrazoxane, and ondansetron.

The compounds of the invention can be administered alone or in combination with other anti-tumor agents, including cytotoxic/antineoplastic agents and anti-angiogenic agents. Cytotoxic/anti-neoplastic agents are defined as agents which attack and kill cancer cells. Some cytotoxic/anti-neoplastic agents are alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents are antimetabolites for tumor cells, e.g., cytosine arabinoside, fluorouracil, methotrexate, mercaptopuirine, azathioprime, and procarbazine. Other cytotoxic/anti-neoplastic agents are antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents are mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents are well known to those of skill in the art. Suitable anti-angiogenic agents for use in the methods and compositions of the invention include anti-VEGF antibodies, including humanized and chimeric antibodies, anti-VEGF aptamers and antisense oligonucleotides. Other known inhibitors of angiogenesis include angiostatin, endostatin, interferons, interleukin 1 (including alpha and beta) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with the compositions of the invention include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. In one embodiment, the anti-cancer drug is 5-fluorouracil, taxol, or leucovorin.

The present invention has multiple aspects, illustrated by the following non-limiting examples.

### 8. EXAMPLES

### Example 1

### In Vivo Expression of Plasmid Encoded IgG for PD-1 or LAG-3 by Synthetic DNA as a New Tool for Cancer Immunotherapy

Cancers employ various strategies to escape immune surveillance including the exploitation of immune checkpoints. Immune checkpoints are receptors found on immune and stromal cells whose function can impact the duration or potency of an immune response. Tumor cells often upregulate ligands for these receptors to protect themselves from the host immune response. Monoclonal antibody (MAb) therapeutics which block immune checkpoint-ligand interactions restore T cell destruction of cancer cells in vivo. MAbs that target the inhibitory T cell signaling mediated by CTLA-4 and/or PD-1 have recently gained regulatory approval for the treatment of some cancers based on remarkable clinical outcomes.

The results presented herein focus on a new method to improve MAb delivery through direct engineering of MAb in the form of synthetic DNA plasmids. This technology can improve many aspects of such a therapy by lowering cost, increasing in vivo expression times and allowing for simple combination formulations in the absence of a host anti-vector immune response, extending use of these groundbreaking therapies to disadvantaged patient populations.

The results demonstrate that “enhanced and optimized” DNA plasmid technology can be used to direct in vivo production of immunoglobulin heavy and light chains of established monoclonal antibodies which can target the immune checkpoints LAG3 and PD-1 as determined in flow cytometry, ELISA and western blot assays. Both antibodies are produced at physiologically relevant levels in blood and other tissues of mice using electroporation-enhanced delivery of DNA plasmids encoding genes for each antibody. Serum antibodies from inoculated animals retain the ability to bind to their targets and are bioactive in vivo and exhibit immune stimulatory effects for host T cells. These studies have significant implications for prophylactic and therapeutic strategies for cancer and other important diseases.

Construction of PD-1, PD-L1. LAG-3, GITR, CD40, OX40, CTLA-4, TIM-3, and 4-1BB dMAb Plasmids and Confirmation of In Vitro and In Vivo IgG Production

DNA monoclonal antibody (dMAb) plasmids were constructed by cloning the sequences for the heavy and light chains of human monoclonal antibodies into the pVAX1 plasmid.

TABLE 1
Sequences

| SEQ ID | Identifier |
|---|---|
| SEQ ID NO: 1 | Anti-hPD-1 optimized nucleic acid sequence (DNA) |
| SEQ ID NO: 2 | Anti-hPD-1 optimized amino acid sequence (protein) |
| SEQ ID NO: 3 | Anti-hLAG3 nucleic acid sequence (DNA) |
| SEQ ID NO: 4 | Anti-hLAG3 amino acid sequence (protein) |
| SEQ ID NO: 5 | DMab TIM-3 nucleic acid sequence (DNA) |
| SEQ ID NO: 6 | Human TIM-3 (protein) |
| SEQ ID NO: 7 | DMab Human PD-1 nucleic acid sequence (DNA) |
| SEQ ID NO: 8 | Human PD-1 (protein) |
| SEQ ID NO: 9 | Mouse LAG3-IgG1 (DNA) |
| SEQ ID NO: 10 | Mouse Lag3-IgG1 (protein) |
| SEQ ID NO: 11 | Human 4-1BB-IgG1 (DNA) |
| SEQ ID NO: 12 | Human 4-1BB-IgG1 amino acid (protein) |
| SEQ ID NO: 13 | Human OX40-IgG1-Agonist (Humanized from mouse |
| | anti-human OX-40) (DNA) |
| SEQ ID NO: 14 | Human OX-40-IgG1 (protein) |
| SEQ ID NO: 15 | Human GITR (Clone 36E5) (DNA) |
| SEQ ID NO: 16 | Human GITR (protein) |
| SEQ ID NO: 17 | Human CD40(Clone #G12) (DNA) |
| SEQ ID NO: 18 | Human CD40(Clone #G12) (protein) |
| SEQ ID NO: 19 | Human CD27 (Agonistic) (DNA) |
| SEQ ID NO: 20 | Human CD27 Amino Acid Sequence (protein) |
| SEQ ID NO: 21 | Tremelimumab (full-length) (DNA) |
| SEQ ID NO: 22 | Tremelimumab (full-length) Amino Acid Sequence |
| | (protein) |
| SEQ ID NO: 23 | Tremelimumab (frame) (DNA) |
| SEQ ID NO: 24 | Tremelimumab (frame) Amino Acid Sequence |
| | (protein) |
| SEQ ID NO: 25 | Ipilimumab (full-length) (DNA) |
| SEQ ID NO: 26 | Ipilimumab (full-length) Amino Acid Sequence |
| | (protein) |
| SEQ ID NO: 27 | Ipilimumab (frame) (DNA) |
| SEQ ID NO: 28 | Ipilimumab (frame) Amino Acid Sequence (protein) |

Supermatants from plasmid-transfected 293T cells were collected at 48 hours post transfection and levels of human IgG assayed using enzyme-linked immunosorbent assay (ELISA).

Nu/J mice (n=4, PD-i or n=5, LAG-3) were injected with 100 μg plasmid followed by electroporation (EP). Sera was collected from mice for up to 35 days and ELISA used to quantitate human IgG levels.

In Vivo Produced IgG Following PD-1 or LAG-3 dMAB Plasmid Administration Bind Specifically to their Targets

Dilutions of sera from mice injected with pVAX1, PD-1 dMAb, or LAG-3 dMAb plasmids were evaluated in a binding ELISA using recombinant PD-1 or LAG-3 protein. Specific binding of PD-1 dMAb and LAG-3 dMAb to recombinant PD-1 or recombinant LAG-3 protein was evaluated with Western analyses.

PHA-stimulated T lymphocytes were incubated with sera from pVAX1 or dMAb plasmid-injected mice followed by fluorophore-conjugated anti-human IgG secondary antibody. Stained cells were evaluated by flow cytometry with gating on live, CD3+ cells. Commercial anti-PD1 and anti-LAG-3 antibodies were used as positive controls.

LAG-3 dMAb Impedes Tumor Growth, Improves Survival, and Promote a Less Inhibitory Tumor Microenvironment.

Cohorts of female C57BL/6 mice were implanted subcutaneously in their right flank with 5x105 B16 F10 melanoma cells and subsequently injected 5 days later with empty pVAX1 or LAG-3 dMAb plasmid. Caliper measurement of tumors and mouse survival were evaluated up to one month post tumor implantation.

To elucidate the role of LAG-3 dMAb in regulatory T cell (Treg)-mediated immune suppression, flow cytometry was used to analyze the population of LAG3+FoxP3+CD25+Treg cells in the tumor and in peritumoral tissues 23 days after inoculation of B16 melanoma cells into C57BL/6 mice.

Plasmids encoding the genetic sequence of antibodies targeting the immune checkpoint molecules were able to direct in vitro and in vivo antibody production.

Human anti-PD-1, anti-LAG-3, anti-GITR, and anti-4-1BB dMAbs produced in mice bound specifically to their targets.

Anti-LAG-3 dMAbs were able to impede tumor growth, improve survival, and promoted a less inhibitory tumor microenvironment in a B16 melanoma tumor challenge model.

DNA plasmids delivered intramuscularly with electroporation can drive robust in vivo antibody production and provides a serology-independent, cost-effective platform for delivering monoclonal antibody therapeutics targeting cancer, infectious diseases, and other conditions.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A composition for generating a synthetic antibody in a subject comprising one or more nucleic acid molecules encoding one or more antibodies or fragments thereof, wherein the one or more antibodies or fragments target at least one immune checkpoint molecule.

2. The composition of claim 1, wherein the at least one immune checkpoint molecule is selected from the group consisting of PD-1, LAG-3, PD-L1, GITR, CD40, OX40, CTLA-4, TIM-3, 4-1BB, and a combination thereof.

3. The composition of claim 1, comprising a nucleotide sequence encoding a cleavage domain.

4. The composition of claim 1, comprising a nucleotide sequence encoding a variable heavy chain region and a variable light chain region of the antibody.

5. The composition of claim 1, comprising a nucleotide sequence encoding a constant heavy chain region and a constant light chain region of human IgG1κ.

6. The composition of claim 1, comprising a nucleotide sequence encoding a polypeptide comprising a variable heavy chain region of the antibody; a constant heavy chain region of human IgG1κ; a cleavage domain; a variable light chain region of the antibody; and a constant light chain region of IgG1κ.

7. The composition of claim 1, wherein the nucleotide sequence encodes a leader sequence.

8. The composition of claim 1, comprising a nucleotide sequence encoding an amino acid sequence having at least about 90% identity over the entire length of at least one amino acid sequence selected from the group of SEQ ID NOs: 2, 4, 6, 8, 10, 12, 14, 16, 18, 20, 22, 24, 26, and 28.

9. The composition of claim 1, comprising a nucleotide sequence having at least about 80% identity over the entire length of the nucleic acid sequence of at least one nucleic acid sequence selected from the group of SEQ ID NOs: 1, 3, 5, 7, 9, 11, 13, 15, 17, 19, 21, 23, 25, and 27.

10. The composition of any one of claims 1-9, wherein the one or more nucleic acid molecules are engineered to be in an expression vector.

11. The composition of claim 1, further comprising a nucleotide sequence encoding an antigen.

12. The composition of claim 11, wherein the antigen is a cancer antigen

13. The composition of any of claims 1-11, further comprising a pharmaceutically acceptable excipient.

14. A method of treating a disease in a subject, the method comprising administering to the subject the composition of any of claims 1-13.

15. The method of claim 13, wherein the disease is cancer.

16. A method for increasing an immune response in a subject in need thereof, the method comprising administering the composition of any one of claims 1-13 to the subject.

17. The method of claim 16, wherein administering the composition comprises an electroporating step.

18. A method of increasing an immune response in a subject in need thereof by administering a combination of a synthetic antigen and an immune checkpoint inhibitor, wherein the immune checkpoint inhibitor is a synthetic antibody, wherein the administering step comprises:
administering to the subject a prime vaccination and a boost vaccination of synthetic antigen, and
subsequent to the boost vaccination, administering to the subject an immune checkpoint inhibitor.

19. The method of claim 18, further comprising a step of administering to the subject a subsequent boost vaccination of the synthetic antigen.

20. The method of claim 18, wherein any of the administering steps include delivering electroporation to the site of administration.
